SEQUENCE LISTING (94 sequences)

SEQ ID NO: 1 - DNA, 1389 bp, Mus musculus
atgcagctgagaaaaatgcagaccatcaaaaaggagcccgcacccctagatcctaccagcagctcagacaagatgctgctgctgaactctgccttagctgaggtggccgaggacctagcctcaggtgaagatttgctcctgaacgaagggagcatggggaaaaacaaatcctcggcgtgtcggagaaaacgggaattcattccggacgagaagaaagacgccatgtattgggagaaacggcggaaaaacaacgaagctgccaaaagatctcgggagaagcgccgcctcaatgacctggttttggagaacaagctgattgccctgggagaagaaaatgccactttaaaagctgagctgctctccctgaaattaaagtttggtttaattagctccacggcgtatgcccaagaaatccagaaactcagtaattccacagctgtctactttcaggactaccagacatccaaggctgccgtgagctcttttgtggacgagcatgagcctgcgatggtagccggaagttgcatctcagtcatcaagcactctccccagagctcgctctccgatgtgtcagaggtgtcctcggtggagcacactcaggaaagccccgcacagggaggctgccggagccctgagaacaagttccctgtgatcaagcaggagcccgtggagttggagagctttgccagggaggccagggaggagcggggcacgtattccacctccatctaccagagctacatgggaagctctttctccacttactcccactccccacccctcttgcaggtccatgggtccactagcaactccccaagaacctcagaggccgatgagggtgtagtgggcaagtcttctgatggggaagacgaacaacaggtccctaagggccccatccattctccagtggagctgcaacgggttcacgccacggtggtgaaggttccggaagtgaacccttctgccttaccgcacaagcttcggattaaagccaaggccatgcaggtcaaagtggaggctttggacagcgagtttgaaggcatgcagaaactctcttcacccgccgatgcgatcgccaaaagacattttgacctggagaaacatggaacctcgggtatggcccattcctccctccctcctttctcagtgcaggtgacgaacattcaagattggtccctcaaatcggaacactggcatcacaaagaactgagcagcaaaactcagagtagcttcaaaacaggtgtggtggaagtcaaagacggtggctataaggtttccgaagctgagaatttgtatttgaagcagggaatagcaaacttatctgcagaggtggtctcgctcaagagattcatagccacacaaccgatctcggcttcggactccaggtaa

SEQ ID NO: 2 - protein, 462 aa, Mus musculus
MQLRKMQTIKKEPAPLDPTSSSDKMLLLNSALAEVAEDLASGEDLLLNEGSMGKNKSSACRRKREFIPDEKKDAMYWEKRRKNNEAAKRSREKRRLNDLVLENKLIALGEENATLKAELLSLKLKFGLISSTAYAQEIQKLSNSTAVYFQDYQTSKAAVSSFVDEHEPAMVAGSCISVIKHSPQSSLSDVSEVSSVEHTQESPAQGGCRSPENKFPVIKQEPVELESFAREAREERGTYSTSIYQSYMGSSFSTYSHSPPLLQVHGSTSNSPRTSEADEGVVGKSSDGEDEQQVPKGPIHSPVELQRVHATVVKVPEVNPSALPHKLRIKAKAMQVKVEALDSEFEGMQKLSSPADAIAKRHFDLEKHGTSGMAHSSLPPFSVQVTNIQDWSLKSEHWHHKELSSKTQSSFKTGVVEVKDGGYKVSEAENLYLKQGIANLSAEVVSLKRFIATQPISASDSR

SEQ ID NO: 3 - DNA, 1704 bp, Mus musculus
tgtccgctctgcctcccacacctagcaccccagcccgctgctgccccggtgagaacccccagcttgggccttgtcatggtgccagcaggtggccctgagcttctgacaggggcctgcctatagacctgcaggcctgaggcctcagactcacactcaaggggcaagaggccctggtggcccacctaagagccacctctgtccccagccctgctgccccactgatgtctgactgagacccagcagtgaccctgagctgcctgcccactgcctcctcctggtccctgaggttggctctgccgaggacggacgactcttctgaagcaggcggctaacggaagcagccccaagcctccaccgcagcatgggcagtgccagcccaggcctgagcaacgtgtcccccggttgcctgctactgttcccagatgtggcaccacgaacagggacggagaaggcagcatcaggagcaatgggccctgagaagcaggaatggagtcctagtccacccgccacccctgagcagggcctgtctgctttctacctctcttactttaacatgtatcccgacgatagcagctgggtcgccaaagtccccgaggcccgtgccggggaggaccacccggaggagcccgagcagtgtcccgtcattgacagccaggcctctgggagcacgttggatgagcactcgctagagcaggtgcaatcgatggttgtgggcgaggtcctgaaagatattgagacggcctgcaagcttctgaacatcacagcagaccctggggactggagccctggtaacgtgcagaagtggcttttatggacagaacaccagtaccggctgcctccagcaggcaaggccttccaggagctgggcggtaaggagctgtgcgccatgtccgaggaacagttccgtcagcgtgcacccttgggtggggatgtactgcatgcccacctggacatctggaagtcagcggcctggatgaaggagaggacctcgcctgggacccttcactactgcgcctccaccagcgaggagggctggacggatggtgaggtggactcgtcgtgctccgggcagcccattcacctgtggcagttcctgaaagaactgctgctcaagccccacagctatggccgcttcatccgctggctcaacaaggagaaaggcatcttcaaaattgaggactcagcacaggtggcccgactgtggggtgtgcgcaagaaccggccagccatgaactatgataaactaagccgctccatccgccagtattacaagaagggcatcattcgtaaacccgacatctctcagcgccttgtctaccaatttgtgcatccagtctgagagccacagagaccagaggcctacaacctgccccaggcagccactctctggttggcctggtcctctctgctcactctgaattcaggggctgctggtatcccagaacccaaggtcccagatagacagccactgatctagggatacacatgagctctctgggtcatacacaggccccaggaagatcgagggagctagttcagcacacagggactggaccaagtcagctcaccggacagtgatgtcactggtctctgctcctgccacaatcctgtaccatatctggcatggtgctaagagatgtctgtaccctgcgttgggaagccaggggtgccctggggatggataataaagacgtaagataactg

SEQ ID NO: 4 - protein, 325 aa, Mus musculus
MGSASPGLSNVSPGCLLLFPDVAPRTGTEKAASGAMGPEKQEWSPSPPATPEQGLSAFYLSYFNMYPDDSSWVAKVPEARAGEDHPEEPEQCPVIDSQASGSTLDEHSLEQVQSMVVGEVLKDIETACKLLNITADPGDWSPGNVQKWLLWTEHQYRLPPAGKAFQELGGKELCAMSEEQFRQRAPLGGDVLHAHLDIWKSAAWMKERTSPGTLHYCASTSEEGWTDGEVDSSCSGQPIHLWQFLKELLLKPHSYGRFIRWLNKEKGIFKIEDSAQVARLWGVRKNRPAMNYDKLSRSIRQYYKKGIIRKPDISQRLVYQFVHPV

SEQ ID NO: 5 - DNA, 4701 bp, Mus musculus
cgggtcgacccacgcgtccgcccacgcgtccggcggagcttctgggttgcgggccgaaacggcaagcggatggagggcgctcgaacggccaggtgtcgtgattaaattagtcagccctcagagacaggcgtcctacctcctttatccagacctcaaaagccccgttgtgcacccgtggtggcttcttcaccttccctgtttcgtcctccactgtatggcccagacatgagtggtcccctagaaggggccgatgggggaggagaccccaggcccggagaacctttttgtcctggaggagtcccatcccctggggccccgcagcaccggccttgtccaggccccagcctggctgatgacactgatgcaaacagcaatggctcaagtggcaatgagtccaacggacccgagtccaggggcgcatctcagcggagttctcatagttcctcttctggcaatggcaaggactcagctctgctggagaccactgagagcagcaagagtacaaactcacagagcccatccccacccagcagctccattgcctacagcctcctgagtgcgagctcagagcaggacaacccatctaccagtggctgcagcagtgaacagtcagctcgagccaggacccagaaagaactcatgactgcacttcgggagctcaaacttcgactgccaccagagcgtcggggcaagggccgctctgggaccttggccacactgcagtacgctctggcctgtgtcaagcaggttcaggctaaccaggaatattaccagcagtggagtctggaggagggtgagccttgtgccatggacatgtctacttacaccctggaggaattggagcatatcacatccgaatacacacttcgaaaccaggacaccttctctgtggctgtgtccttcctgacaggccggattgtctatatttcggagcaggcaggtgtcctgctgcgttgcaaacgggatgtgtttcggggtgcccgcttctcagagctcctggctccccaggatgtgggtgtcttctatggctctactacaccatctcgactgcccacctggggcactggcacctctgcaggttcaggtctcaaggacttcacccaggaaaagtctgtcttctgccgaatcagaggaggtcctgaccgggatccagggcctcggtaccagccattccgcctaaccccatatgtgaccaagattcgggtctcagatggagcccctgcacagccgtgctgcctactcattgccgagcgcatccactctggttatgaagctccccggatccctcctgacaagaggatcttcaccacccgacacacaccaagctgcctcttccaggatgtagatgaaagggctgccccactgctgggttaccttccccaggatctcctgggggctccagtacttctctttctacatcctgaggaccgacccctcatgctggccattcataagaagatactgcagctggcaggccagccctttgaccattcccctattcgcttctgtgctcggaacggggaatatgtcaccatggacaccagctgggccggttttgtgcacccctggagccgcaaggtggctttcgtgttgggtcgccataaagtgcgcacggcacccctgaatgaggacgtcttcactcccccagcccccagcccagctccgtccctggactctgatatccaggagctctcagagcagatccatcgattgctgctgcagcctgtgcacagctccagccccacggggctctgtggagttggccctctgatgtcccctggtcctctacacagccctggctcctccagtgatagcaatgggggggacgctgaggggcctgggcctcctgctccagtgactttccagcagatctgtaaggatgtgcatctggtaaagcaccagggacaacagctcttcattgaatctcgggccaagcccccaccccggccccgcctccttgctacaggtacattcaaagccaaagtccttccctgccagtccccaaaccccgaactggaggtggccccagttcctgaccaagcctcgttagccttggcccctgaggagccagagaggaaagaaacctctggctgttcctaccagcagatcaactgcctggacagcatcctcaggtatttggagagctgcaacattcccagtacaaccaagcgtaaatgtgcctcctcctcctcctacactgcctcttcagcctctgatgatgacaagcagagggcaggtccagttcctgtgggggccaagaaagatccgtcgtcagcaatgctgtctggggagggggcaactcctcggaaggagccagtggtgggaggcaccctgagcccgctcgccctggccaataaggcagagagcgtggtgtccgtcaccagtcagtgtagcttcagctccaccatcgtccatgtgggagacaagaagcccccggagtcggacatcatcatgatggaagacctgcctggcctggcccctggcccagcccccagtccggcccccagccccacagtagcccctgacccaaccccagatgcttatcgcccagtgggtctgaccaaggccgtgctgtccctgcacacacagaaggaagagcaagccttcctcaaccgcttcagagatcttggcaggcttcgtggacttgacacctcttctgtggccccctcagcccctggctgccaccatggccccattccccctggtcgccgacaccactgccgatctaaagcaaagcgttcccgccaccaccaccaccagaccccccggcccgaaactccctgctatgtctcccatccttcacctgtgccctcttctggaccctggccacccccaccagccacgacccccttcccagcaatggtccagccctacccactcccagtattctcccctcgaggaggaccccagccccttccccctgcccctacatctgtgtcccctgctaccttcccttctcccttagtgaccccaatggtggccttggtgctccctaactatctattccctaccccacctagttatccatatggggtgtcccaggcccctgttgaggggccacccacgcctgcttcccactcgccctctccatccctgcccccaccacctctcagccccccccaccgcccagactccccactgttcaactcgagatgcagctccccactccagctcaatctgctgcagcttgaggagtccccccgcacggaggggggcgctgctgcaggaggcccaggaagcagtgctgggcccctgcctcccagtgaggagactgctgagccagaggccagattggtggaggttactgagtcgtccaatcaggatgcactttcaggctccagcgacctgctggagctactgctccaagaagactctcgctcgggcacaggctccgcagcctcaggctccctgggctctggcctgggctctgggtctggttcaggatcccacgaagggggaagcacctcagccagcatcacccgcagcagtcagagcagccatacaagcaagtactttggcagcatcgactcttccgaggctgaagctggggctgctcgggccaggactgagcctggggaccaggtcattaagtgtgtgctccaggaccccatctggctgctcatggccaatgccgaccagcgtgtcatgatgacataccaggtgccgtccagggatgcagcctctgtgctgaagcaagaccgggagaggctccgggccatgcagaaacagcagccacggttctcagaggaccagaggcgggaactgggtgctgtgcactcctgggtccggaagggccagctgcctcgggcccttgatgtgatggcgtgtgtggactgtggcagcagcgttcaagatcctggccactctgatgacccgctcttctcagaactggatggattggggctggagcccatggaagagggtggaggcgagggtggtgggtgtggtgttggcggtggtgggggtgatggtggtgaggaggcccagacccaaattggggctaagggttcaagctctcaggactctgccatggaggaagaagagcaaggtgggggctcatccagcccagctttacctgcagaagaaaacagcaccagctagatccattttggggccgcttacagcagtctaatgagaggcttcctttcgaccatgttggggttcttataactcaagatacagctggaccaaccaataggaaactgccccagcttctcccaacatagggggctggacccccattaccagcccaggcacaggagctgcctctagcttcttagcagagtggaagttctcagccccatttggaggattgtccacgcccgtcccactgaggagacgggcgggtcttcggttaaggttgctgacaagctgctgaagtggtctgtccaaatcccagctgagcctgagtcccagtcgcagggttggggctgcacttatttatttgggagagacagctcactctcccacctcaccccaagatgggaggaggggaacctgggatctgtgtaggatccaggtccgtgaacccctagctgctccagggtgggggaggttggtggaccatggagtccctggtgctgcccctcaggtgggacccaggtgttctcagctctaccctctaccaatgacatttgtgtttttgatattgtgtctgttattttttttttaatacaaaatgacaaaatgaaaaaccaaaaa

SEQ ID NO: 6 - protein, 1291 aa, Mus musculus
MSGPLEGADGGGDPRPGEPFCPGGVPSPGAPQHRPCPGPSLADDTDANSNGSSGNESNGPESRGASQRSSHSSSSGNGKDSALLETTESSKSTNSQSPSPPSSSIAYSLLSASSEQDNPSTSGCSSEQSARARTQKELMTALRELKLRLPPERRGKGRSGTLATLQYALACVKQVQANQEYYQQWSLEEGEPCAMDMSTYTLEELEHITSEYTLRNQDTFSVAVSFLTGRIVYISEQAGVLLRCKRDVFRGARFSELLAPQDVGVFYGSTTPSRLPTWGTGTSAGSGLKDFTQEKSVFCRIRGGPDRDPGPRYQPFRLTPYVTKIRVSDGAPAQPCCLLIAERIHSGYEAPRIPPDKRIFTTRHTPSCLFQDVDERAAPLLGYLPQDLLGAPVLLFLHPEDRPLMLAIHKKILQLAGQPFDHSPIRFCARNGEYVTMDTSWAGFVHPWSRKVAFVLGRHKVRTAPLNEDVFTPPAPSPAPSLDSDIQELSEQIHRLLLQPVHSSSPTGLCGVGPLMSPGPLHSPGSSSDSNGGDAEGPGPPAPVTFQQICKDVHLVKHQGQQLFIESRAKPPPRPRLLATGTFKAKVLPCQSPNPELEVAPVPDQASLALAPEEPERKETSGCSYQQINCLDSILRYLESCNIPSTTKRKCASSSSYTASSASDDDKQRAGPVPVGAKKDPSSAMLSGEGATPRKEPVVGGTLSPLALANKAESVVSVTSQCSFSSTIVHVGDKKPPESDIIMMEDLPGLAPGPAPSPAPSPTVAPDPTPDAYRPVGLTKAVLSLHTQKEEQAFLNRFRDLGRLRGLDTSSVAPSAPGCHHGPIPPGRRHHCRSKAKRSRHHHHQTPRPETPCYVSHPSPVPSSGPWPPPPATTPFPAMVQPYPLPVFSPRGGPQPLPPAPTSVSPATFPSPLVTPMVALVLPNYLFPTPPSYPYGVSQAPVEGPPTPASHSPSPSLPPPPLSPPHRPDSPLFNSRCSSPLQLNLLQLEESPRTEGGAAAGGPGSSAGPLPPSEETAEPEARLVEVTESSNQDALSGSSDLLELLLQEDSRSGTGSAASGSLGSGLGSGSGSGSHEGGSTSASITRSSQSSHTSKYFGSIDSSEAEAGAARARTEPGDQVIKCVLQDPIWLLMANADQRVMMTYQVPSRDAASVLKQDRERLRAMQKQQPRFSEDQRRELGAVHSWVRKGQLPRALDVMACVDCGSSVQDPGHSDDPLFSELDGLGLEPMEEGGGEGGGCGVGGGGGDGGEEAQTQIGAKGSSSQDSAMEEEEQGGGSSSPALPAEENSTS

SEQ ID NO: 7 - DNA, 1897 bp, Mus musculus
gaattcggcacgagcagcgagacgccgcgcacggtgcttccccagtggagccaatcggctaacccgcgctccggcagagtccttggcgctcgcccgccggcgggacagaccacccgcctctggccgctctctggaccctggccgccccgagcgaagactggagcaaaatgatgcttcaacatccaggccaggtctctgcctcagaagtcagtgcgaccgccattgtcccctgcctctcacctcctgggtcactggtatttgaggattttgctaacctgacaccctttgtcaaggaagagctgagattcgccatccagaataaacacctctgccatcggatgtcctctgcgctggagtcagttaccgtcaacaacagacccctggagatgtcagtcaccaagtctgaggcggcccctgaagaagatgagaggaaaaggaggcggcgagaaagaaataaaattgctgctgccaagtgtcgaaacaagaaaaaggagaagacagagtgcctgcagaaagagtcagagaaactggagagtgtgaatgctgagctgaaggcccagattgaggagctgaagaatgagaaacagcatttgatatacatgctcaacctgcaccggcccacctgtatcgtccgggctcagaatggacggacaccggaagacgagaggaacctctttatccaacagataaaagaaggaacattgcagagctaagcagaggtggcacggaggcaattggggagttcttactgaatcctccttttccaccccacaccctgaagccattggaaaactggcttcctgtgcacttctagaatcccagcagccaagagccgttggggcaggagggcctgtggtgacctactgcattgacccactctgcccccgagtgaaccgtggagcaggcaggagcatcctttgtctcaccaattccaggatttaggccttatcatcccggccagtctcagatgacctagctggccccaggctggggtcctatgcaaagcaggatcccactaatgggattcaggcagaagtgtctaccttgataggtggggtgggaccacatcctccactgtggctgacaacgcccttccaagggaatatggaatgagaacattcattattgaggttgtccaatggccagggtatgctttctagaaaatatgctgttctgtcccagaatgactgtgcatagggtatccgtttcagagcctggtgttgtgctatttagatgtttgtcttgcacaacattggcatgatttttccgggagtttcatcagatctgatttctgagagtctggggatctgccatggtggaaagtgcccctcaaaagcatttgtgtggccacatgaactggctggcaccaggggagtgaaactggctgatgaccagctgagccactttgtgccaacagaggatggacgacacctttccctgtacccactgcagaggaagaaccctgggcacagcagctttgtccttggctacaaactgttacaacgtcacacaatgaaggcacaaagtccaactttcaaagggtgtaggactccatactcagtgacagggcaggaagagccaaagataaccacagccacagcctgtggagaccagggttggaagccaggtgcagggccaggcatctgcattgtgggatgttaatggcacttttgtcttgtagctattttgagatgtggtccagagcatttcagctgggagatctccctctggccaccaggactctggctactgttaaaatcctgatgtttctgtggaatcctcagtgtttaatcccactcaatagtatcattacagttttctgtaagagaaaatattacttatttatcccagtattcctagcctgtcaacataataaatatcggaacaaaacctggta

SEQ ID NO: 8 - protein, 181 aa, Mus musculus
MMLQHPGQVSASEVSATAIVPCLSPPGSLVFEDFANLTPFVKEELRFAIQNKHLCHRMSSALESVTVNNRPLEMSVTKSEAAPEEDERKRRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 9 - DNA, 1038 bp, Mus musculus
cccagagataagctgacgctgggcaaacccctgggggaaggttgcttcgggcaagtagtcatggctgaagcagtgggaatcgataaagacaaacccaaggaggcggtcaccgtggcagtgaagatgttgaaagatgatgccacagagaaggacctgtctgatctggtatcagagatggagatgatgaagatgattgggaaacataagaacattatcaacctcctgggggcctgcacgcaggatggacctctctacgtcatagttgaatatgcatcgaaaggcaacctccgggaatacctccgagcccggaggccacctggcatggagtactcctatgacattaaccgtgtccccgaggagcagatgaccttcaaggacttggtgtcctgcacctaccagctggctagaggcatggagtacttggcttcccaaaaatgtatccatcgagatttggctgccagaaacgtgttggtaacagaaaacaatgtgatgaagatagcagactttggcctggccagggatatcaacaacatagactactataaaaagaccacaaatgggcgacttccagtcaagtggatggctcctgaagccctttttgatagagtttacactcatcagagcgatgtctggtccttcggggtgttaatgtgggagatctttactttagggggctcaccctacccagggattcccgtggaggaactttttaagctgctcaaagagggacacaggatggacaagcccaccaactgcaccaatgaactgtacatgatgatgagggattgctggcatgctgtaccctcacagagacccacattcaagcagttggtcgaagacttggatcgaattctgactctcacaaccaatgaggaatacttggatctcacccagcctctcgaacagtattctcctagttaccccgacacaagtagctcttgttcttcaggggacgattctgtgttttctccagaccccatgccttatgaaccctgtctgcctcagtatccacacataaacggcagtgttaaaacatga

SEQ ID NO: 10 - protein, 345 aa, Mus musculus
PRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPTNCTNELYMMMRDCWHAVPSQRPTFKQLVEDLDRILTLTTNEEYLDLTQPLEQYSPSYPDTSSSCSSGDDSVFSPDPMPYEPCLPQYPHINGSVKT

SEQ ID NO: 11 - DNA, 2429 bp, Mus musculus
acccacgcgtccggccggtttcactgctcccctcagtctcttttgggctctttccgggcatcgggacgatgaccgtcaaagccgaggctgctcgaagcacccttacctactccagaatgaggggaatggtagcgattctcatcgcttttatgaaacagagaaggatgggcctgaacgattttattcagaagattgccagcaacacctatgcatgcaaacacgctgaagttcagtccattttgaaaatgtcccatcctcaggagccggagcttatgaacgctaacccctctcctccgccaagtccctctcaacaaatcaacctgggtccgtcctccaaccctcacgccaaaccctccgactttcacttcttgaaagtgatcggaaagggcagttttggaaaggttcttctggctaggcacaaggcagaagaagtattctatgcagtcaaagttttacagaagaaagccatcctgaagaagaaagaggagaagcatattatgtcagagcggaatgttctgttgaagaatgtgaagcaccctttcctggtgggccttcacttctcattccagaccgctgacaaactctactttgtcctggactacattaatggtggagagctgttctaccatctccagagggagcgctgcttcctggaaccacgggctcgattctacgcagctgaaatagccagtgccttgggctatctgcactccctaaacatcgtttatagagacttaaaacctgagaatattctcctagactcccaggggcacatcgtcctcactgactttgggctctgcaaagagaatattgagcataacgggacaacatctaccttctgtggcacgcctgagtatctggctcctgaggtcctccataagcagccgtatgaccggacggtggactggtggtgtcttggggctgtcctgtatgagatgctctacggcctgcccccgttttatagccggaacacggctgagatgtacgacaatattctgaacaagcctctccagttgaaaccaaatattacaaactcggcaaggcacctcctggaaggcctcctgcagaaggaccggaccaagaggctgggtgccaaggatgactttatggagattaagagtcatattttcttctctttaattaactgggatgatctcatcaataagaagattacacccccatttaacccaaatgtgagtgggcccagtgaccttcggcactttgatcccgagtttaccgaggagccggtccccagctccatcggcaggtcccctgacagcatccttgtcacggccagtgtgaaggaagcagcagaagccttcctcggcttctcctatgcacctcctgtggattccttcctctgagtgctcccgggatggttctgaaggacttcctcagcgtttcctaaagtgttttccttaccctttggtggaggttgccagctgacagaacattttaaaagaatttgcacacctggaagcttggcagtctcgcctgcccggcgtggcgcgacgcagcgcgcgctgcttgatgggagctttccgaagagcacaccctcctctcaatgagcttgtgaggtcttcttttcttctcttccttccaacgtggtgctagctccaggcgagcgagcgtgagagtgccgcctgagacagacaccttggtctcagttagaaggaagatgcaggtctaagaggaatccccgcagtctgtctgagctgtgatcaagaatattctgcaatgtgccttttctgagatcgtgttagctccaaagctttttcctatcgcagagtgttcagtttgtgtttgtttgtttttgttttgttttgtttttcccttggcggatttcccgtgtgtgcagtggcgtgagtgtgctatgcctgatcacagacggttttgttgtgagcatcaatgtgacacttgcaggacactacaatgtgggacattgtttgtttcttccacatttggaagataaatttatgtgtagactgttttgtaagatatagttaataactaaaacctattgaaacggtcttgcaatgacgagcattcagatgcttaaggaaagcattgctgctacaaatatttctatttttagaaagggtttttatggaccaatgccccagttgtcagtcaaagccgttggtgttttcattgtttaaaatgtcacctataaaacgggcattatttatgttttttttccctttgttcatattcttttgcattcctgattattgtatgtatcgtgtaaaggaagtctgtacattgggttataacactagatatttaaacttacaggcttatttgtaaaccatcattttaatgtactgtaattaacatgggttataatatgtacaattcctcctccttaccacacaactttttttgtgtgcgataaaccaattttggtttgcaataaaatcttgaaacct

SEQ ID NO: 12 - protein, 431 aa, Mus musculus
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNGTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHIFFSLINWDDLINKKITPPFNPNVSGPSDLRHFDPEFTEEPVPSSIGRSPDSILVTASVKEAAEAFLGFSYAPPVDSFL

SEQ ID NO: 13 - DNA, 2447 bp, Mus musculus
gcagggcgggtgagagcgccgtgaaagccgcggaacgccgtgcacctccgcgactctactacggcaagctagtccggacgggtcgtcgtccccgcgcgccaccagcccttggtgaaacgacagggagcgtccggcttccccagcaccgccctgcgagactcaaaacagccacaccgcaaagcgagcctcgggcggaaggaggcggagcttcaggcggccccgcctccgcggaaggatacacatctccgtggtccaaaaccccggggcgaggcggccggggcgtgtgagctgctcggccagctgccgtctacgcgctttcgcgcggccaccgggcaactgcgccgcgcggctgccccgctgagcgctcggcctcggggccgtgggatccgccgcgctgtctgcggtcaggaagaccgccctcccgcgtccttgccggacgggtcagaggcggcaccgcacgcgaggccacccgcgatgctgctgtccaagttcggctccctggcgcacctctgcgggcctggcggcgtggaccacctcccagtgaagatcctacagccagccaaggctgacaaggagagcttcgagaaggtgtaccaggtgggcgccgtgctgggcagcggcggcttcggcacggtctacgcgggcagccgcatcgccgacggactcccggtggctgtgaagcacgtggtgaaggagcgggtgaccgagtggggcagtctcggcggagtggccgtgcccctggaggtggtgctgctgcgcaaggtgggcgcggcgggcggcgcgcgcggcgtcatccgcttgctggactggttcgagcggcccgacggcttcttgttggtgctggagcgacccgagccggcacaggacctcttcgacttcatcactgaacgaggcgccctggacgagccgctggcgcgtcgcttcttcgcgcaggtgcttgccgctgtgcggcactgccacaattgtggggtcgtgcaccgcgacatcaaggacgagaacctgctggtggacctgcgctcgggagagctgaagctcatcgacttcggctcgggcgcggtgctcaaggacacggtctacactgactttgatggcacccgtgtgtacagccccccagagtggatccgatatcaccgatatcacgggcggtctgccactgtgtggtctctgggtgtactgctctacgacatggtgtgtggggacattccctttgagcaggatgaggagatcttgcgcggcaggctctttttccggaggagggtctccccagagtgccagcagcttattgagtggtgtctctccctgaggccctcagagaggccctccctggaccaaattgctgcccacccctggatgctggggacagaggggagcgttccagagaactgtgaccttcggctttgtgccctggatactgacgacggagccagtaccacttccagcagtgagagcttgtgaggaggagaaggggcctgggctcggcctagccagcgctctcccagaattgaacactttctgcctgggatgtctgctgcaaaagcagtgacctctgacccctggtgacctttgctctcggcaccgggcctgtttcctttgctttgagtgcctttttgaacgctgctccacagggcctgggttttcttgagctcttctgtccaaagatggctgagggctaagcaaggtcctgccctgggtggatacttgaaccagagatcccgaccctgctgctccatctcaggaggcagccttcctgaccaagtgtgtttgacatggagcgccctgtggtgcccacctccaaccctccagtctcctggtgttcatctgggcatgtctgcacaagcaatgcaacgctgggccactgctgcccgtctgcctccccggcacggcacggctccgcacgcaacctaagcgtgccaccacggtctcttatttatggtgtgatcaccctggagggcgcccccgccctgctggggctatttattgtttaatttatttgctgaggttcctccaagcaaccaccttctccaggcccctggggtgttgaaagtcaaatgtggctgttgagtccacagacccccatcctaattcctgcacctggaggagttccccaacccccgtgtttgcgggaggaagcatttgtacagtggctaatttaaggggagtgggagaccctgtcaccctgagcactctgcgctggggaggggtttaaattattgaccttgtacagtctgcttgctggctctgaaagctggggttgggggacagagtctcaagcccttaatttattttagcagctgtgtttctgtgaccctggtgtgactaagcatcaggggtggggttgtataagttcaaaagtgtgaaatgtctgaagatcatattttttatacaggtatttcaattaaatgttttggtatataatggaaaaaaaaaaaaaaaaaaaaaaaaaaaaa

SEQ ID NO: 14 - protein, 326 aa, Mus musculus
MLLSKFGSLAHLCGPGGVDHLPVKILQPAKADKESFEKVYQVGAVLGSGGFGTVYAGSRIADGLPVAVKHVVKERVTEWGSLGGVAVPLEVVLLRKVGAAGGARGVIRLLDWFERPDGFLLVLERPEPAQDLFDFITERGALDEPLARRFFAQVLAAVRHCHNCGVVHRDIKDENLLVDLRSGELKLIDFGSGAVLKDTVYTDFDGTRVYSPPEWIRYHRYHGRSATVWSLGVLLYDMVCGDIPFEQDEEILRGRLFFRRRVSPECQQLIEWCLSLRPSERPSLDQIAAHPWMLGTEGSVPENCDLRLCALDTDDGASTTSSSESL

SEQ ID NO: 15 - DNA, 2299 bp, Mus musculus
cctgggccccgccgcggacgcgcggagccgcctgggccgcgccggaggagggcggggagaggaccatgtgaatgtgctccggagctgagcgccaagccaagcagtgtttgaaaggaacaggatgctgatctaatcgtggcaaaaagtcagtccgaccgctggtttcgaagacatgtggtgtatataaagtttgtgatagttggtggaaatttgggagcttggataatgggctgtgtgcaatgtaaggataaagaagcagcgaaactgacagaggagagggacggcagcctgaaccagagctctgggtaccgctatggcacagaccccacccctcagcactaccccagcttcggcgtgacctccatcccgaactacaacaacttccacgcagctgggggccagggactcaccgtctttgggggtgtgaactcctcctctcacactgggaccctacgcacgagaggagggacaggagtgacactgtttgtggcgctttatgactatgaagcacggacggaagatgacctgagttttcacaaaggagaaaaatttcaaatattgaacagctcggaaggagattggtgggaagcccgctccttgacaaccggggaaactggttacattcccagcaattacgtggctccagttgactccatccaggcagaagagtggtactttggaaaacttggccgcaaagatgctgagagacagctcctgtcctttggaaacccaagaggtacctttcttatccgcgagagccaaaccaccaaaggtgcctactcactttccatccgtgattgggatgatatgaaaggggaccacgtcaaacattataaaatccgcaagcttgacaatggtggatactatatcacaacgcgggcccagtttgaaacacttcagcaactggtacagcattactcagagaaagctgatggtttgtgttttaacttaactgtggtttcatcaagttgtaccccacaaacttctggattggctaaagatgcttgggaagttgcacgtgactcgttgtttctggagaagaagctggggcaggggtgtttcgctgaagtgtggcttggtacctggaatggaaatacaaaagtagccataaagacccttaagccaggcaccatgtctccggagtccttcctggaggaggcgcagatcatgaagaagctgaagcatgacaagctggtgcagctctacgcggtcgtgtctgaggagcccatttacatcgtcacggagtacatgagcaaaggaagtttgcttgacttcttaaaagatggtgaaggaagagctctgaagttgccaaaccttgtggacatggcggcacaggttgctgcaggaatggcttacatcgagcgcatgaattatatccacagagatctgcgatcagcaaacattctagtggggaatggactaatttgcaagattgctgactttggattggctcggttgattgaagacaatgaatacacagcaagacaaggtgcgaagtttcccattaagtggacagcccccgaagcggccctgtatggaaggttcacaatcaagtctgacgtatggtcttttggaatcttactcacagagctggtcaccaaaggaagagtgccatacccaggcatgaacaaccgggaggtgctggagcaggtggagagaggctataggatgccctgcccacaggactgcccgatctccctgcacgagctcatgatccactgctggaaaaaggatccggaagagcgcccgaccttcgagtacttgcagggcttcctggaggactactttacggccacagagccccagtatcagcccggtgaaaacctgtgagagcctgcgcttcagacgcctcttcccgaggcctccctacccctccccattagcttccaattctgtagccagctgccccagagcaggagaaccgtccaggatcagattgcatgtgactcttgaagctgaacttccacggccctcattaatgacacttgtcccccagtccgaacctcctctgtgaaccatctgagacagaagcgtgttatttctcagacttggaaatgcattgtatcgatgttatgtcaaaggccaaacctctgttcagtgtaaatagctgctcctgtgccaacaatcccagtgctttccttttttaaaaaagaaaaagcaaatcctatgtgattttaactctgatttcacctgattcaactaaaaaaaaaaaagtattattttccaaaagtggcctctttgtctaaaacaataaaattttttttcatgttttaacaaaaaaaaaaaaaaaaaaaaaaaa

SEQ ID NO: 16 - protein, 534 aa, Mus musculus
MGCVQCKDKEAAKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESQTTKGAYSLSIRDWDDMKGDHVKHYKIRKLDNGGYYITTRAQFETLQQLVQHYSEKADGLCFNLTVVSSSCTPQTSGLAKDAWEVARDSLFLEKKLGQGCFAEVWLGTWNGNTKVAIKTLKPGTMSPESFLEEAQIMKKLKHDKLVQLYAVVSEEPIYIVTEYMSKGSLLDFLKDGEGRALKLPNLVDMAAQVAAGMAYIERMNYIHRDLRSANILVGNGLICKIADFGLARLIEDNEYTARQGAKFPIKWTAPEAALYGRFTIKSDVWSFGILLTELVTKGRVPYPGMNNREVLEQVERGYRMPCPQDCPISLHELMIHCWKKDPEERPTFEYLQGFLEDYFTATEPQYQPGENL

SEQ ID NO: 17 - DNA, 2804 bp, Mus musculus
ggacgtcagactagagagtagggagagagactggtgctcgagggacagggctagcccggacgcgtgtccgcgcctcggaggtggcaagtaggcagtgtcgggtggcgaggcaacgatggagctcctgcggactatcacctaccagccggccgccggcaccaagatgtgcgagcaggctctgggcaaagcttgcggcggggactcaaagaagaagcgaccacagcagccttctgaagatgggcagccccaagcccaggtgaccccggcggccccgcaccaccatcaccaccattcccactcgggacccgagatctcgcggattatagtcgaccccacgacggggaagcgctactgccggggcaaagtgctgggcaagggtggatttgcaaagtgttacgaaatgacagatctgacaaacaacaaagtctacgctgcaaaaattattcctcacagcagagtagctaaacctcatcagagggaaaagatcgacaaagaaatcgagcttcacagactactgcaccataagcatgtcgtgcagttttaccactactttgaagacaaagaaaacatttacattctcttggaatactgcagtagaaggtccatggctcacatcttgaaagcaagaaaggtgttgacagagccagaagtccgatactacctcaggcagattgtgtcaggactcaagtatcttcacgaacaagaaatcttgcacagggatctcaagctagggaactttattattaatgaagccatggagctgaaggtgggagactttggtttggcagccagactggaaccactggaacacagaaggagaacaatatgtggaaccccaaattatctctcccccgaagtcctcaacaaacaaggacacggctgtgaatcagacatctgggccttaggctgtgtaatgtatacgatgctgctaggaagacctccattcgaaaccacaaatctgaaagaaacgtacaggtgcataagggaagcaaggtataccatgccgtcctcattgctggcccctgctaagcacttgatagctagcatgctgtccaaaaacccagaggaccgccccagtttggatgacatcattcggcatgacttcttcctgcagggtttcactccggacagactctcttccagctgttgccacacagttccagatttccacttgtcaagcccagccaagaatttctttaagaaagccgcagccgctctttttggtggcaagaaggacaaagcaagatataacgacacacacaataaggtgtctaaggaagatgaagacatttacaagcttcggcatgatttgaagaaagtgtcgataacccagcagcctagcaaacacagagcagacgaggagccccagccgcctcccactactgttgccagatctggaacgtccgcagtggaaaacaaacagcagattggggatgcaatccggatgatagtcagggggactctcggcagctgcagcagcagcagcgaatgccttgaagacagcaccatgggaagtgttgcagacacagtggcaagagtccttcgaggatgtctagaaaacatgccggaagctgactgtatccccaaagagcagctgagcacgtcctttcagtgggtcaccaagtgggtcgactactccaacaaatatggctttgggtaccagctctcggaccacactgttggcgtccttttcaacaacggggctcacatgagcctccttccggacaaaaagacagttcactattatgcggaacttggccaatgctctgttttcccagcaacagatgcccctgaacaatttattagtcaagtgacggtgctgaaatacttttctcattacatggaggagaacctcatggatggtggtgatctcccgagtgttactgacattcgaagacctcggctctacctcctgcagtggttaaagtctgataaagccttaatgatgctcttcaatgacggcacatttcaggtgaatttctaccacgatcatacaaaaatcatcatctgtaaccagagtgaagaataccttctcacctacatcaatgaggacaggatctctacaactttcagactgacgactctgctgatgtctggctgttcgttagaattgaaaaatcgaatggaatatgccctgaacatgctcttacagagatgtaactgaaaacattattattattattattataattatttcgagcggacctcatgggactcttttccactgtgagatcaacagggaagccagcggaaagatacagagcatgttagagaagtcggacaggtggtggtacgaatacaattcctctgtggcctgctggactgctggaaccagaccagcctaaggtgtagagttgactttggacaatcctgagtgtggagccgagtgcagttttccctgagatacctgtcgtgaaaaggtttatgggacagtttttcagaaagatgcattgactctgaagttctctctgttgagagcgtcttcagttggaagacttggaactgtgaatacacttcctgaaggggagggagaagggaggttgctcccttgctgtttaaaggctacaatcagagcagcttttggctgcttaactgtgaactatggccatacatttttttttttttggttatttttgaatacacttgtggttggaaaagtgcattccttgttaataaactttttatttattacagccccaagagcagtatttattatcaagatgttctctttttttatgttgaccatttcaaactcttggcaataaagagtatgacatagaaaaaaaaaaaaaaaaaaaaaaaaa

SEQ ID NO: 18 - protein, 682 aa, Mus musculus
MELLRTITYQPAAGTKMCEQALGKACGGDSKKKRPQQPSEDGQPQAQVTPAAPHHHHHHSHSGPEISRIIVDPTTGKRYCRGKVLGKGGFAKCYEMTDLTNNKVYAAKIIPHSRVAKPHQREKIDKEIELHRLLHHKHVVQFYHYFEDKENIYILLEYCSRRSMAHILKARKVLTEPEVRYYLRQIVSGLKYLHEQEILHRDLKLGNFIINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYNDTHNKVSKEDEDIYKLRHDLKKVSITQQPSKHRADEEPQPPPTTVARSGTSAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICNQSEEYLLTYINEDRISTTFRLTTLLMSGCSLELKNRMEYALNMLLQRCN

SEQ ID NO: 19 - DNA, 658 bp, Mus musculus
aacttagctggactgcagccttctccgctggaactcgccaagccagctgatttccccatccaaagccatgaagagcggcgtatgtctgtgcgtggtgatggcagtcctagctgctggcgccctggcgcagccggtagtccctgcagaagctacggaccccgtggagcagcgggcgcaagaggcgccccgaaggcagctgcgggctgtgctccggacggacggcgagccccgagcgcgcctgggcgcactgctagcgcgatacatccagcaggtccgcaaagctccttctggccgcatgtccgttcttaagaacctgcagagcctggaccccagccatagaataagtgaccgggactacatgggctggatggattttggccggcgcagtgccgaggactacgaatacccatcgtagtgggccagcgtcttggccctgcttggaggaggtggaatgaggaaacaaccacacatacgacccctcgcctctaatgtctgacgttttgagtatctatttattaagtccccaatgtgaaatctgtccagagtgtgcaatgcagccacatctcagcctagctgtgtggtcggaaggcagtgtttccttcagtgactcccagacctaatgttgctatgctattaaagagatttccttctgcccccc

SEQ ID NO: 20 - protein, 115 aa, Mus musculus
MKSGVCLCVVMAVLAAGALAQPVVPAEATDPVEQRAQEAPRRQLRAVLRTDGEPRARLGALLARYIQQVRKAPSGRMSVLKNLQSLDPSHRISDRDYMGWMDFGRRSAEDYEYPS

SEQ ID NO: 21 - DNA, 1381 bp, Mus musculus
cttggtgacactagacagagcaactccagcgttaccgctcccgctcctggtttctcggcttctcatcgcagtcaatcttggactttggggttttgctactgtcagaaggacttctttctgcttcaagtgcttgacaacgcacccctttatcagggtatcagagcatcgccacagaatgaagctggtttccatcaccctgatgttattgggttcactcgctttcctaggcgcggacactgcagggccagatactccttcgcagttccgaaagaagtggaataagtgggcgctaagtcgtgggaagagggaactacaagcatccagcagctaccctacgggactcgctgatgagacgacagttcctacccagactcttgatccattcctggacgagcagaacacaactggccccctacaagccagcaatcagagcgaagcccacattcgtgtcaaacgctaccgccagagcatgaaccagggttcccgcagcaatggatgccgcttcgggacctgcacatttcagaaattggcccaccagatctaccagctaacagacaaagacaaggacggcatggctcccagaaacaagatcagccctcaaggctatggccgccggcgccggcgttccctgctggaggtcctccggtcccggactgtggagtcctcccaggagcagacacacacagccccaggcccctgggcgcacatctccagactctttaggatataggtgcgggtgacagcattgaacagtcgggcgagtatcccgttggcgcctgcggaatcagagaacttcgcaccggggcggactgagacaatcctgcagagatctgcctggctgcccctaggggaggcagaggaacccaagaccaagccaggctcatgccagaaaccgagacttacaggctgatactctccgggcaggggtctgagccactgccttgcccgctcataaactggtttctcacggggcataagcctcattactacttgaactttccaaaacctagcgaggaacgtgcaatgcttgttgtccagccaaaggtaactatagtatttaagtttgttgctgtcaaggtttttttttttgtaacttcaaatatatagagatatttttgtacgttatatattgtattaagggcattttaaagtgattatattgtcaccttcccctattttaagacgtgaatgtctcagcaaggtgtaaggttgtttggttccgtgtgtgtgtgtgtgtgtgtgtgtgtgtgtgtgtgtgtgtaaggtggagagcgcctgattatcgcctgtggatgaagaaaaaacattgtgtttcctataatctatttacataaaatatgtgatctgggaaaaagcaaaccaataaactgtctcaatgctg

SEQ ID NO: 22 - protein, 184 aa, Mus musculus
MKLVSITLMLLGSLAFLGADTAGPDTPSQFRKKWNKWALSRGKRELQASSSYPTGLADETTVPTQTLDPFLDEQNTTGPLQASNQSEAHIRVKRYRQSMNQGSRSNGCRFGTCTFQKLAHQIYQLTDKDKDGMAPRNKISPQGYGRRRRRSLLEVLRSRTVESSQEQTHTAPGPWAHISRLFRI

SEQ ID NO: 23 - DNA, 850 bp, Mus musculus
tcgagcggccgcccgggcaggtccaggatcaagagtcaccgcttcgcaagcactgcctggctccatcaggatccccgcaggctcagctccaaggcaccgctcaccaggaaggcatcatgggcttcctgaagttctcccctttcctggttgtcagcatcttgctcctgtaccaggcatgcagcctccaggcagtgcctttgaggtcaatcttggaaagcagcccaggcatggccactctcagtgaagaagaagttcgcctgctggctgcactggtgcaggactatatgcagatgaaagccagggagctggagcaggaggaagagcaggaggctgagggctctagcttggacagccccagatctaagcggtgtgggaatctgagtacctgcatgctgggcacgtacacacaagacctcaacaagtttcacaccttcccccaaacttcaattggggttgaagcacctggcaagaaaagggatgtggccaaggacttggagacaaaccaccaatcccattttggcaactaagctccttctctcctttctagtttccttcttgctttcttcctataacttgatgcatgtagttcctctctggttgctctccaggctattactggttgctttcctgaggcaaagaatggtatctgaaatccccagtgggtgaggagaaagtcccacaggctaaaagagaatcacccaggaagatggcagagagcaagggcacactcaggaagatggcagagagcaagggcagtcatctggcttcctagtagagcttctagtcttgcttctggaagtgttggttgtttgggaaataaaactattttttaaaaaaaaaaaaaaaaaaa

SEQ ID NO: 24 - protein, 136 aa, Mus musculus
MGFLKFSPFLVVSILLLYQACSLQAVPLRSILESSPGMATLSEEEVRLLAALVQDYMQMKARELEQEEEQEAEGSSLDSPRSKRCGNLSTCMLGTYTQDLNKFHTFPQTSIGVEAPGKKRDVAKDLETNHQSHFGN

SEQ ID NO: 25 - DNA, 2912 bp, Mus musculus
aaaggcagcctgataaagctccttgtgacaggctgtcttgccagtctcccagtatgctcctcttgctctgaagtgctccaggattgaaaccacagcttcccaaattagcctgggaagagtgtgcggacccagcagccttttaacccgcgtcagtgcctttgctatgttcaagactgctgttttggatggtgaatgctagctagcactccatcgagacatgacagcaaaaaattctccaaaagaatttactgcttcggaatctgaggtttgcataaagactttcaaggagcagatgcgcttggaacttgagcttccaaagctaccaggaaacagacctacatctcccaaaatttctccacgcagttcaccaaggaattcaccatgctttttcagaaagttgctggtgaataaaagcatccgacagcggcgtcgcttcacggtggctcatacatgctttgatgtggaaaatggcccttctccaggtcggagcccactggaccctcaagccggctcttcgtcgggactggtacttcatgccgcctttcctgggcacagccagcgcagggagtcgttcctctacgatcttgacagcgactatgacttgtcaccaaaagcgatgtccaggaactcatcacttcccagtgagcaacacggcgatgacctgattgtcactccttttgcccaggttcttgccagcttgcgaagtgtaagaaacaacttcaccctgctgacgaaccttcatggagcgccgaacaagaggtcaccagcggctagtcaggctccagtctccagagtcagcctgcaagaggaatcatatcagaaactagcaatggagacgctggaggaactagactggtgcctagaccagctagagaccatccagacctaccgctctgtcagcgagatggcttcaaacaagttcaaaaggatgctgaaccgggagctgacacacctctcagagatgagcagatcagggaaccaggtgtctgagtacatttcaaacacgttcttagacaagcagaacgatgtggaaatcccatctcccacgcagaaggacagggagaagaagaagaagcagcagctcatgacccagataagtggagtgaagaaactgatgcacagctcaagcctgaacaacacaagcatctcacgcttcgggatcaacacggaaaatgaggatcatctagccaaggagctggaagacctgaacaaatggggccttaacatcttcaatgtggctgggtactcacataatcggccccttacgtgcatcatgtatgcaatattccaggaaagagaccttctgaagacgtttaaaatctcatctgacacctttgtaacctacatgatgactttagaagaccattaccattctgatgtggcatatcacaacagcctgcatgctgctgacgtggcccagtcaactcacgttctcctttctacgccggcactggatgctgtcttcacagacctggaaatcctggctgccatttttgcagctgccatccatgatgtcgatcatcctggagtctccaatcagtttctcatcaatacaaattctgaacttgctttgatgtataatgatgaatctgttctggaaaaccatcaccttgctgtgggattcaaattgctacaagaggaacactgcgacatctttcagaatcttaccaagaagcaacgccagacactcaggaaaatggtgattgacatggtgttggcaactgatatgtccaaacacatgagcctcctggcagaccttaaaacaatggtagaaaccaagaaggtgacaagctccggtgttctcctcctggacaactatactgaccggatacaggttcttcgcaacatggtacactgtgcagacctgagcaaccccaccaagtccttggaattgtatcggcaatggaccgatcgtatcatggaggagtttttccagcagggagacaaagaacgggagaggggaatggagattagcccaatgtgtgataagcacacagcttctgtggaaaaatcccaggttggtttcattgactacattgtccatccactgtgggagacctgggcagacctggttcaaccggatgctcaagatattctggatacactagaagataacaggaactggtaccagagtatgataccccagagcccttccccgccactggatgagaggagcagggactgccaaggcctgatggagaagtttcagtttgaactgacccttgaggaagaggattctgagggaccggaaaaggagggagaaggccacagctatttcagcagcacaaagacgctttgtgtgattgatccagagaacagggattctctggaagagactgacatagacattgcaacagaagacaagtctccgatcgacacataatctctctccctctgtgtggagatgaacattccacccttgactgagcatgcccgctgagtggtagggtcacctaccatggccaaggcctgcacaggacaaaggccacctggcctttccagttacttgagtttggagccagaatgccaggccgtgaagcaaatagcagttccatgctgtcttgccttgcctgcaagcttggcggagacccgcagctgtatgtggtagtagaggccagttcccatcaaagctaaaatggcttgaaaacagaggacacaaagctgagagattgctctgcactaggtgttgggaagctgtcctgacagatgactgaactcactaacaacttcatctataaatctcaccacccaacccattgtctgccaacctgtgtgcctttttttgtaaaatgttttcgcgtctttgaaatgcctgttgaatatctagagtttagtaccaacttctacaaacttttttgagtctttcttgaaaaacaaaaaaaaaaaaaaaaaaaaaaaaaaaaaaaaaaaaa

SEQ ID NO: 26 - protein, 721 aa, Mus musculus
MTAKNSPKEFTASESEVCIKTFKEQMRLELELPKLPGNRPTSPKISPRSSPRNSPCFFRKLLVNKSIRQRRRFTVAHTCFDVENGPSPGRSPLDPQAGSSSGLVLHAAFPGHSQRRESFLYDLDSDYDLSPKAMSRNSSLPSEQHGDDLIVTPFAQVLASLRSVRNNFTLLTNLHGAPNKRSPAASQAPVSRVSLQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGINTENEDHLAKELEDLNKWGLNIFNVAGYSHNRPLTCIMYAIFQERDLLKTFKISSDTFVTYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFQNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDERSRDCQGLMEKFQFELTLEEEDSEGPEKEGEGHSYFSSTKTLCVIDPENRDSLEETDIDIATEDKSPIDT

SEQ ID NO: 27 - DNA, 1240 bp, Mus musculus
ggccgcgcgggagtctgagaatgcaaagtgccatgttcctggctgtccagcacgactgcgtacccatggacaagagtgcaggcaacggccccaaggtcgaggagaagcgggagaaaatgaagcggacactcttaaaccattggaagacccgtttgagctacttcttgcagaattcctctgctcctgggaagcccaaaactggcaagaaaagcaaacagcaaacttttatcaagccttctcctgaggaagcgcacgtctgggcagaagcatttgatgaactgctggccagtaaatatgggctggctgcattcagggcgtttttaaagtccgagttctgtgaagaaaacattgaattctggttggcttgtgaagacttcaaaaaaaccaaatcaccccaaaaactgtcctcaaaagcaaggaaaatctataccgacttcatagagaaggaagctcccaaagagataaacatagacttccaaacgaaatctctgattgcccaaaatatccaagaggctacaagtggctgcttcaccacagctcagaagagggtgtacagtttgatggagaacaattcttatcctcggttcttggagtccgaattctaccaggacttatgtaaaaagccacagatcaccacggagccccatgctacatgagaccaggagtccccccacacacaaaggacattccattctgtctcccaagagcaaaggctgtgacctgccagaaaaaaaaaaaaaactgaccttgaattcagcctgagtgttaggaaaacatcgctcagaactattgattcaatgttgggtagtgaatcaggaagtcagcaacctaggagaggctctgtgtgagaacggcttccctcactgtgtgaagaacagagggagggaacaggcctctgaatgtgttcttcctccttgtcgggaaagcagagtttgagatgaaagatccgatgcaatgttgttggagcatttaaaatcaagaggtctgggattatgtggccttagctagttggctgtacaccttccctaaactagtccatgttacacatagtggtgttagttctagttttaatattttagtactaagtaacattacaatgtttactgtgtgcaagggtgttgacgttcttaggactacagatcattagtactagtgtgtcacgtatcactgaaactgagaagtatgtttgagttgttaaatggtgtgtgtgatggaccgaatgctgtgccgtgctgtagaa

SEQ ID NO: 28 - protein, 211 aa, Mus musculus
MQSAMFLAVQHDCVPMDKSAGNGPKVEEKREKMKRTLLNHWKTRLSYFLQNSSAPGKPKTGKKSKQQTFIKPSPEEAHVWAEAFDELLASKYGLAAFRAFLKSEFCEENIEFWLACEDFKKTKSPQKLSSKARKIYTDFIEKEAPKEINIDFQTKSLIAQNIQEATSGCFTTAQKRVYSLMENNSYPRFLESEFYQDLCKKPQITTEPHAT

SEQ ID NO: 29 - DNA, 2345 bp, Mus musculus
aaaaagtatatgaggacaaatgtaaggcaaatgaccatggaaacagttgaatcacagcaggatcgaagtgtaacacgttctgtggcagagcatagctctgctcatatgcagactggtcaaatttctgttcctactctagctcaggtagcaacaattgcagagacagatgattctgcagactcagaagtaattgattcgcataaacgtagagaaattctttcacgaagaccctcatatagaaaaatactgaatgaactttcctctgatgtgcctggtattcccaagattgaagaagaaaaatcagaggaagaagggacaccacctaacattgctaccatggcagtaccaactagcatatatcagactagcacggggcaatacaatgaggagactgaccttgccccaagtcacatggctgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgcccgggagtgtcgcaggaagaagaaagaatatgtcaaatgtcttgaaaatcgtgtggctgtgcttgaaaatcaaaacaagaccctcattgaggaactcaaggccctcaaagacctttattgccataaagcagagtaactgtgtttgatttggaccttgttgactgtgaactctaatcggggcaggcgatgcagcatcctcataatggccatgtggacttgtagatgggtctcttaacccttgcttaagaatacagtctgctgtagagtgtgaattgggaatactgttccatgggttggaatgcagctcccctcacattaccaagcttgctctattgccaatagcatgcaacatatgttttgtttgcccttctgcttctacttttttcagggaagctgctaaagaatgtcgacgtcgaaagaaagagtatgtgaagtgtcttgagagtcgagtcgcagtgctggaagttcagaacaagaagcttatagaggagcttgaaactttgaaagacatttgctctcccaaaacagattagtagaaatatttaactatgaactgattacagcatgtacagttgcttttgaatgcaatacaaatatatagccggcaagaattatggctttttcctttgtatcattcatctaactttctaaaactaacattcctaagatgctttgttgtatttaatttgctcttacctctaaggtcaattttttagaagagacaaactcaaaaaatgtatgtaacaaattcttaaaatgaagtatttgtaagacttgttccagtcaacatatttacagttcccagtctctctgtcatgaatagtgtcctatgcaataaaaattttgcaggttttaagaatcattttaggaaagggtgaatcaaaggcagtgcatctctccagtagtaagataaaatcaacccatagagatacctcaggaaagaatgaaaggaagtgtatcctgatgacatgacgtgagaatagcctacaaatgaatttatgcatttatagatttttataatcgtcactttgtaaagaaagtattgtattgctgtccttgggtgccacagttgaagacagttttaaatagaaccatgttggttgctctttgtactatttggtatttatttaagtatctgagcatttactacagcttcctactatgtatgtagtatgtgaatttctacaaaagtttgtgctctttgctgttatttaatgaaagagacaacatattttcattatctggaatgagttccacaagtatgaatttattgctacactggatcagcagccttgcaaatactgggccatttcattagaggacaacagcagggctctaggagcagagttcagtgtggagcacttgcctggcatgctacatgttcagttgaaagggaagacctcaagctctgcaaatggaatggggtccaggggaagaggttagaggttagcctttgtgctgtactaggcttcttgctgatcgtctggagagtttctgctgatgaccctccattgtgaattcttgcaacctcaggaatgttaacgtttaaaaaacttcccaagatgtcatttttgattttacaacttggatcaattttgttttgctctttggaatatagctgtgtacatttgtcacgtaggtttaggctggccttaaactcacagttctcttgcctcagccttctgagtgcttggattacggatgtgggccagaatatccagtttgatcaagtattcttttataaaatattactttcttttt

SEQ ID NO: 30 - protein, 229 aa, Mus musculus
MTMETVESQQDRSVTRSVAEHSSAHMQTGQISVPTLAQVATIAETDDSADSEVIDSHKRREILSRRPSYRKILNELSSDVPGIPKIEEEKSEEEGTPPNIATMAVPTSIYQTSTGQYNEETDLAPSHMAAATGDMPTYQIRAPTTALPQGVVMAASPGSLHSPQQLAEEATRKRELRLMKNREAARECRRKKKEYVKCLENRVAVLENQNKTLIEELKALKDLYCHKAE

SEQ ID NO: 31 - DNA, 9848 bp, Mus musculus
gctgaagcgtttcctcaagcctgccggggtgggaggagaggaggaggtggtggtggtggaggaggtggaggcagagggtggagagagagaaagcgcacgccgagaggaggtgtgggtgttccgctcccatcctaacggaacgagctccctcttcgcggacatgggattgcccagcggctgctaacccctctcctggtcctgatcccccaaaccggcgtggctccccggtcaccaaggagctgattacaagggaccaggatttgcatccttggctgggcgtccattggctacagagtgcctgacctgggtcaggctttccaacacggacatgtctgacaaaatgtcgagtttcctacatattggagacatttgttctctgtatgcggagggatctacgaatggatttatcagcaccttaggcttggttgatgaccgttgtgttgtacagccagaagccggggaccttaacaatccacccaagaaattcagagactgcctctttaagctatgtcctatgaatcgatactccgcacagaaacagttctggaaagctgctaagcccggggccaacagcactacagatgcagtgctgctcaacaaattgcatcatgctgcagacttggaaaagaagcagaatgagacagaaaacaggaaattgttggggaccgtcatccaatatggcaacgtgatccagctcctgcatttgaaaagcaataaatacctgactgtgaataagaggctcccagccttgctagagaagaatgccatgagggtgacgttggacgaggctggaaatgaagggtcctggttttacattcaaccattttacaagcttcgctccatcggagacagtgtggtcataggcgacaaggtagttttgaatcctgtcaatgctggccagcctctacatgccagcagtcatcagctggtggataacccaggctgcaatgaggtcaactccgtcaactgtaatacaagctggaagatagtgcttttcatgaaatggagtgataacaaagacgacattctcaaaggaggtgatgtggtgaggctcttccatgccgagcaagagaagtttctcacctgtgatgagcaccggaagaagcagcatgtgttcctgaggaccaccggcaggcagtcagccacgtcggccaccagttctaaagccctgtgggaagtggaggtagtccagcacgacccatgtcggggtggagctgggtactggaatagcctcttccggttcaagcacctggctacagggcattacttggctgcagaggtagaccctgactttgaggaagaatgcctggagtttcagccctcagtggaccctgatcaggatgcatctcggagtaggttgagaaacgcgcaagaaaaaatggtatactctctggtctccgtgcctgaaggcaacgacatctcctccatctttgagctagaccccacgactctgcgtggaggtgacagccttgtcccaaggaactcctatgtccgtctcagacacctgtgcaccaacacctgggtacacagcacaaacatccccatcgacaaggaagaggagaagcctgtgatgctgaaaattggtacctctcccctgaaggaggacaaggaagcatttgccatagttcctgtttcccctgctgaggttcgggacctggactttgccaatgatgccagcaaggtgctgggctccatcgctgggaagttggaaaagggcaccatcacccagaatgagagaaggtctgtcacgaagcttttggaagacttggtttactttgtcacgggtggaactaactctggccaagacgtgcttgaagttgtcttctctaagcccaatcgagagcggcagaagctgatgagggaacagaatattctcaagcagatcttcaagctgttgcaggcccccttcacggactgcggggatggcccgatgcttcggctggaggagctgggggatcagcgccatgctcctttcagacatatttgccgactctgctacagggtcctgcgacactcacagcaagactacaggaagaaccaggagtacatagccaagcagtttggcttcatgcagaagcagattggctatgacgtgctggccgaagacaccatcactgccctgctccacaacaaccggaaactcctggagaagcacatcaccgcggcagagattgacacgtttgtcagcctggtgcgaaagaacagggagcccaggttcttggattacctctctgacctctgcgtatccatgaacaagtcaatccctgtgacacaggagctcatctgtaaagctgtgctcaatcccaccaatgctgacatcctgattgagaccaagctggttctttctcgttttgagtttgaaggcgtttccactggagagaatgctctggaagccggggaggatgaggaagaggtgtggctgttctggagggacagcaacaaagagatccgtagtaagagtgtccgggaattggcgcaagatgctaaagagggacagaaggaagacagggacatcctcagctactacagatatcagctgaacctctttgcaaggatgtgtctggaccgccagtacctggccatcaatgaaatctccgggcagctggatgttgatctcattctccgctgcatgtctgacgagaacctcccctacgacctcagggcatccttttgccgcctcatgcttcacatgcatgtggaccgagatccccaagagcaggtgacacctgtgaaatatgcccgactgtggtcagaaattccctctgagatcgccattgatgactatgacagcagtggaacatccaaagatgaaattaaggagaggtttgcacagacgatggagtttgtggaggagtacctaagagatgtggtttgtcaaagattccccttctctgataaggagaaaaataagctcacgtttgaggttgtgaacttagccaggaatctcatatactttggtttctacaacttttctgaccttctccgattaaccaagatcctcttggcaatcttagactgtgtccatgtgaccactatcttccccattagcaagatgacaaaaggagaagagaataaaggcagtaacgtgatgaggtctatccatggcgttggggagctgatgacccaggtggtgctgcggggaggaggcttcttgcccatgactcccatggctgcggcccctgaaggaaatgtgaagcaggcagagccagagaaagaggacatcatggtcatggacaccaagttgaagatcattgaaatactccagtttattttgaatgtgagattggattataggatctcctgcctcctgtgtatatttaagcgagagtttgatgaaagcaattcccagtcatcagaaacatcctccggaaacagcagccaggaagggccaagtaatgtgccaggtgctcttgactttgaacacattgaagaacaagcggaaggcatctttggaggaagtgaggagaacacacctttggacctggatgaccatggtggcagaaccttcctcagggtcctgctccacttgacaatgcatgactacccacccctggtgtctggggccctgcagctcctctttcggcacttcagccagaggcaggaggtccttcaggccttcaaacaggttcaactgctggttactagccaagatgtggacaactacaaacagatcaagcaagacttggaccaactaaggtccattgtggagaagtctgagctctgggtgtacaaaggccaaggtcccgatgagcctatggacggagcctccggtgaaaatgagcataagaaaaccgaggaggggacgagcaagccactgaagcacgagagcaccagcagctacaactaccgagtggtgaaagagattttgattcgacttagcaagctctgcgtgcaggagagcgcgtcggtgaggaagagccggaagcagcagcaacgactgctgaggaacatgggcgcacacgctgtggtgctggagctgctgcagatcccctacgagaaggccgaagacacaaagatgcaagagatcatgcggctggctcatgaatttttgcagaatttctgtgcaggcaaccagcagaatcaagctttgctgcataaacacataaacctgtttctcaagccagggatcctggaggcagtgacgatgcagcacatcttcatgaacaacttccagctgtgcagtgagatcaacgagagagtggtccagcactttgttcactgcatagagacccacggtcgaaacgtccagtatatcaagtttctccagacgattgtcaaggcagaagggaaattcattaaaaagtgccaagacatggtcatggctgagcttgtcaactctggagaggacgtcctcgtgttctacaatgacagagcctctttccagactctgatccagatgatgcggtccgagcgtgaccggatggatgagaacagccctctcatgtaccacatccatctggtggagctcttggccgtgtgcacagagggcaagaatgtgtacacggagatcaagtgcaactccttgctcccgctcgatgacatcgttcgtgtggtcactcatgaagactgcatccccgaggttaagatcgcttacattaacttcctgaatcactgctatgtggatacggaggtggagatgaaggagatttacacaagcaaccacatgtggaagttgtttgagaatttcctcgtggacatctgcagggcctgtaacaacacaagcgacaggaagcacgcagactccattctggagaagtacgtcactgaaatcgtgatgagcatcgtcaccaccttcttcagctctcccttctcagaccagagcaccactctgcagacccgccagcctgtctttgtgcaactcctgcaaggcgtgttccgagtttaccactgcaactggctgatgccgagccaaaaagcctcggtggagagctgcatccgggtgctctctgacgtagccaagagccgggccatagccattcctgttgacctggacagccaagtcaacaacctcttcctgaagtcccacaacattgtgcagaaaacagccctgaactggcggttatcagcccgaaacgccgctcgcagagactctgtactggcagcatccagagactaccgaaatatcattgagaggttacaggacatcgtgtctgccctagaggaccggctcaggcccctggtgcaggctgagctgtctgtgctcgtggatgttctacacagaccagaactgctcttccccgagaacacggatgccaggaggaaatgtgagagtggaggtttcatctgcaagctaataaaacataccaagcaactgctggaggagaatgaagagaaactatgcattaaagtcttacagaccctcagggaaatgatgaccaaagacagaggctatggagagaagcaaatttccattgatgaatcggaaaatgccgagctgccacaggcaccggaagctgagaactccacagagcaggagcttgaaccaagtccacccctgaggcaactggaagaccataaaaggggtgaggcactccgacaaattttggtcaaccgttactatggaaacatcagaccttcaggaagaagagagagccttaccagctttggcaatggcccactatcaccaggaggacccagcaagcctggtggaggagggggaggtcctggatctagttccacaagcaggggtgagatgagcctggctgaggttcagtgtcacctcgacaaggagggggcctccaacctggtcatcgatctcataatgaatgcatccagtgaccgagtattccatgaaagcattctgctggccatcgcacttctggaaggaggcaacaccaccatccagcactcgtttttctgccggctgacagaagataagaaatcagagaagttcttcaaggttttttacgatcgaatgaaggtggcccagcaggaaatcaaggcgacagtgacagtgaacaccagcgacttgggaaacaaaaagaaagatgatgaagtggacagggatgccccgtctcggaagaaagccaaagagcccacaacacagataacagaagaggtccgggatcagctcctggaagcatctgctgccaccaggaaagcctttaccaccttccggagggaggccgaccctgatgaccattaccagtctggggagggcacccaggctacaaccgacaaagccaaggatgacctagagatgagcgctgtcatcaccatcatgcagcctatcctgcgcttcctgcagctgctgtgtgaaaaccacaaccgagatctgcagaatttccttcgttgccaaaataataagaccaactacaatttggtgtgtgagacactgcagtttctggactgtatttgtgggagcacaaccggaggccttggtcttcttggactgtacataaatgaaaagaatgtagcacttatcaaccaaaccctggagagtctgacggagtactgtcaagggccttgccatgagaaccagaactgcatcgccacccacgagtccaatggcatcgatatcatcacagccctcatcctcaatgatatcaaccctctgggaaagaagcggatggacctggtgttagaactgaagaacaatgcttcgaagctgctactggccatcatggaaagcagacacgatagtgaaaatgcagagaggatcctgtacaacatgaggcccaaggagctggtggaagtgatcaagaaggcctacatgcaaggtgaagtggaatttgaggatggggagaacggtgaggatggagctgcctcacccaggaacgtgggccacaacatctacatcctcgctcaccagttggctcggcataacaaagaacttcaaaccatgctgaaacctggaggccaggtggatggggatgaagctctggagttctacgcgaagcacacagcacaaattgagattgtcagactggaccggacaatggaacagatcgtcttccctgtgcccagcatctgtgaattcctgactaaggaatcgaaacttcgaatatattacaccacagagcgggatgagcaaggtagcaagatcaatgacttcttcctgcgctccgaggacctctttaacgagatgaactggcagaagaaacttcgagcccagcctgtcttgtactggtgtgcccgaaacatgtctttctggagcagcatctccttcaacctggccgtcctgatgaacctgctggtggcgtttttctatccatttaaaggagtgaggggaggaacactagagccacactggtcaggcctcctgtggacagccatgctcatctctctggccattgtcattgctctgcccaagccccacggcatccgggccttaattgcttctacaatcctacgactgatattttcagttgggttgcagcccacactgtttctgctgggagctttcaatgtctgcaataaaatcatcttcctgatgagctttgtgggcaactgtgggaccttcaccagaggctaccgggccatggttctggatgtggagttcctctatcatttgctgtatctactcatctgtgccatgggcctcttcgtacatgagttcttctatagcttgctgctttttgatttagtgtacagagaggagactttgcttaatgtcattaaaagtgtcacccgcaatggacggtccatcatcttgacagcggtcctggctctgatcctggtttacctgttctcaattgtgggctatctgttcttcaaggatgactttatcttggaagtagataggttgcccaatgaaacagctgttccagaaactggcgagagtttggccaacgatttcctgtactctgatgtgtgcagggtagagacgggggagaactgcacctctcctgcacccaaagaagagctgctccctgccgaagaaacggaacaggataaggaacacacgtgtgagaccctgctcatgtgcatcgtcactgttctgagtcacgggctgcggagtgggggaggggtaggagacgtgctcaggaagccatccaaagaggagcctctgtttgctgcaagggtgatctacgacctcctcttcttcttcatggtcatcatcatcgtcctgaacctgattttcggggtcatcatcgacacctttgctgacctgaggagtgagaagcaaaagaaggaggagatcttaaaaaccacgtgcttcatctgcggcttggaaagggacaagtttgacaataagactgtcacctttgaagagcacatcaaggaagaacacaacatgtggcactatctgtgcttcatcgtgctggtgaaagtgaaggactccacagagtacaccgggcctgagagttacgtggcagagatgatcagggaaagaaaccttgattggttcctcagaatgagagccatgtccctggtcagcagcgattctgaaggggaacagaacgagctgaggaacctgcaggagaagctggagtctaccatgaagctggtcaccaatctttctggccagctgtcagaactaaaggaccagatgacagaacagaggaagcagaaacaaagaatcggccttctaggacatcctcctcacatgaatgtcaacccacagcagccggcctaggcaaatgaggcagagggactctgctcagccctctgtatatcactgtcagggtgggtacggctcattggttctgatttgcccactaagggtacatgtgcgcttagtacatttgtaaatactcagttttgtattgtatgtatatgattgctattctcagaggtttggactttcgtattgtaattagctctgttggcatggtgacttgtcactcctgccaaaaatattaaaaatgccttttttggaaggactacagaaagtacctgatttgcacttgaaccagattatagatttaaaagtatatgacatgtattttgtatttaaaactagaatagccagtatttatgttttttataaaactgtgcaatacaaattatgcaatcaccataactttgtaactcctgagtgtcctaagggagtacacatctttgaagctgatttgttgatactcgtgtaataaatggttaaatatcaaatgctgctgctgctgccaaaattatattaatagcgagtttctggcccctgggcaattttgtaccttgtaattatcctatggtgatgctgtttctcgttgctaatggcattagtgcccctgtatcctagtgataactccaggtctgtgaaccattcaaacagcattcattttgagaaaagcaactttagtttcaaggataattttaagcttcaaaattaatcatttaaagtgtttctttaagagagccatgttagaggctcacactttagcttgaaaggagttgatgaattaattttttaaagggaactttttacatgacgtttggaataacagcatattgctgaccagtcagtgtcatctcccgggtgaattttgatgtcacgttatagtcaaatgagttagctgatggtttctagattttcttcctctgaaccatgatgcagtaggtaagaagttattatgcgtatatacatatatacattcatatacgacaaagtaggagctgtccccttaggatgcatagctgcccctagggtacgtagctgaacactgacaatggcgttcttctgaaagagccacgtttgggttttatttctttgtcacatgatttcttttctggatgggtgcaaagtatcacaggaagtgttttctctctgtcgccttgttttgtacctgggtctcgctttactagaccgtctctgcacaaaagtttaaaaactgaaccgtatgcagagttccgaagcaagtcaagtttgtaaatgcatacctaaaaatatttaataaacgatgcagaatcct

SEQ ID NO: 32 - protein, 2749 aa, Mus musculus
MSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSTGENALEAGEDEEEVWLFWRDSNKEIRSKSVRELAQDAKEGQKEDRDILSYYRYQLNLFARMCLDRQYLAINEISGQLDVDLILRCMSDENLPYDLRASFCRLMLHMHVDRDPQEQVTPVKYARLWSEIPSEIAIDDYDSSGTSKDEIKERFAQTMEFVEEYLRDVVCQRFPFSDKEKNKLTFEVVNLARNLIYFGFYNFSDLLRLTKILLAILDCVHVTTIFPISKMTKGEENKGSNVMRSIHGVGELMTQVVLRGGGFLPMTPMAAAPEGNVKQAEPEKEDIMVMDTKLKIIEILQFILNVRLDYRISCLLCIFKREFDESNSQSSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDEPMDGASGENEHKKTEEGTSKPLKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLKPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHNIVQKTALNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKQISIDESENAELPQAPEAENSTEQELEPSPPLRQLEDHKRGEALRQILVNRYYGNIRPSGRRESLTSFGNGPLSPGGPSKPGGGGGGPGSSSTSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQSGEGTQATTDKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQTMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLLICAMGLFVHEFFYSLLLFDLVYREETLLNVIKSVTRNGRSIILTAVLALILVYLFSIVGYLFFKDDFILEVDRLPNETAVPETGESLANDFLYSDVCRVETGENCTSPAPKEELLPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIRERNLDWFLRMRAMSLVSSDSEGEQNELRNLQEKLESTMKLVTNLSGQLSELKDQMTEQRKQKQRIGLLGHPPHMNVNPQQPA

SEQ ID NO: 33 - DNA, 3454 bp, Mus musculus
ggcacgagccgagttggaggaagcagcggcagcggcagcggcagcggtagcggtgaggacggctgtgcagccaaggaaccgggacagcgaagcgacggcaggtcgcagctggatcgcaggagcctgggagctgggagcttcagaggccgctgaagcccaggctgggcagaggaaggaagcgagccgacccggaggtgaagctgagagtggagcgtggcagtaaaatcagacgacagatggacagtgtgacaggaacgtcagagaggattgggcctcgctgcgagagtcagcctggagtcaaggtgttgacaagttgctgagaaggacacgtgggaggacggtggcgcgcggagggagagccctgtcttcagtcaccccgttgatggaggacagatggacagcagccggacggccagtcacctctcttaaacctttggatagtggtcctttgtgctctgctggacacctgttggggattttagcccattctctgaactcactttctcttaaaacgtaaactcggacggcagtgtgcgagccagctcctctgtggcagggcactagagctgcagacatgagtgcagagggctaccagtacagagcactgtacgactacaagaaggagcgagaggaagacattgacctacacctgggggacatactgactgtgaataaaggctccttagtggcacttggattcagtgatggccaggaagcccggcctgaagatattggctggttaaatggctacaatgaaaccactggggagaggggagactttccaggaacttacgttgaatacattggaaggaaaagaatttcaccccctactcccaagcctcggccccctcgaccgcttcctgttgctccgggttcttcaaaaactgaagctgacacggagcagcaagcgttgccccttcctgacctggccgagcagtttgcccctcctgatgttgccccgcctctccttataaagctcctggaagccattgagaagaaaggactggaatgttcgactctatacagaacacaaagctccagcaaccctgcagaattacgacagcttcttgattgtgatgccgcgtcagtggacttggagatgatcgacgtacacgtcttagcagatgctttcaaacgctatctcgccgacttaccaaatcctgtcattcctgtagctgtttacaatgagatgatgtctttagcccaagaactacagagccctgaagactgcatccagctgttgaagaagctcattagattgcctaatatacctcatcagtgttggcttacgcttcagtatttgctcaagcattttttcaagctctctcaagcctccagcaaaaaccttttgaatgcaagagtcctctctgagattttcagccccgtgcttttcagatttccagccgccagctctgataatactgaacacctcataaaagcgatagagattttaatctcaacggaatggaatgagagacagccagcaccagcactgccccccaaaccacccaagcccactactgtagccaacaacagcatgaacaacaatatgtccttgcaggatgctgaatggtactggggagacatctcaagggaagaagtgaatgaaaaactccgagacactgctgatgggacctttttggtacgagacgcatctactaaaatgcacggcgattacactcttacacctaggaaaggaggaaataacaaattaatcaaaatctttcaccgtgatggaaaatatggcttctctgatccattaaccttcaactctgtggttgagttaataaaccactaccggaatgagtctttagctcagtacaaccccaagctggatgtgaagttgctctacccagtgtccaaataccagcaggatcaagttgtcaaagaagataatattgaagctgtagggaaaaaattacatgaatataatactcaatttcaagaaaaaagtcgggaatatgatagattatatgaggagtacacccgtacttcccaggaaatccaaatgaaaagaacggctatcgaagcatttaatgaaaccataaaaatatttgaagaacaatgccaaacccaggagcggtacagcaaagaatacatagagaagtttaaacgcgaaggcaacgagaaagaaattcaaaggattatgcataaccatgataagctgaagtcgcgtatcagtgagatcattgacagtaggaggaggttggaagaagacttgaagaagcaggcagctgagtaccgagagatcgacaaacgcatgaacagtattaagccggacctcatccagttgagaaagacaagagaccaatacttgatgtggctgacgcagaaaggtgtgcggcagaagaagctgaacgagtggctggggaatgaaaataccgaagatcaatactccctggtagaagatgatgaggatttgccccaccatgacgagaagacgtggaatgtcgggagcagcaaccgaaacaaagcggagaacctattgcgagggaagcgagacggcactttccttgtccgggagagcagtaagcagggctgctatgcctgctccgtagtggtagacggcgaagtcaagcattgcgtcattaacaagactgccaccggctatggctttgccgagccctacaacctgtacagctccctgaaggagctggtgctacattatcaacacacctccctcgtgcagcacaatgactccctcaatgtcacactagcatacccagtatatgcacaacagaggcgatgaagcgctgccctcggatccagttcctcaccttcaagccacccaaggcctctgagaagcaaagggctcctctccagcccgacctgtgaactgagctgcagaaatgaagccggctgtctgcacatgggactagagctttcttggacaaaaagaagtcggggaagacacgcagcctcggactgttggatgaccagacgtttctaaccttatcctctttctttctttctttctttctttctttctttctttctttctttctttctttctttctttctttctaatttaaagccacaacacacaaccaacacacagagagaaagaaatgcaaaaatctctccgtgcagggacaaagaggcctttaaccatggtgcttgttaacgctttctgaagctttaccagctacaagttgggactttggagaccagaaggtagacagggccgaagagcctgcgcctggggccgcttggtccagcctggtgtagcctgggtgtcgctgggtgtggtgaacccagacacatcacactgtggattatttcctttttaaaagagcgaatgatatgtatcagagagccgcgtctgctcacgcaggacactttgagagaacattgatgcagtctgttcggaggaaaaatgaaacaccagaaaacgtttttgtttaaacttatcaagtcagcaaccaacaacccaccaacagaaaaaaaaaaaaaa

SEQ ID NO: 34 - protein, 723 aa, Mus musculus
MSAEGYQYRALYDYKKEREEDIDLHLGDILTVNKGSLVALGFSDGQEARPEDIGWLNGYNETTGERGDFPGTYVEYIGRKRISPPTPKPRPPRPLPVAPGSSKTEADTEQQALPLPDLAEQFAPPDVAPPLLIKLLEAIEKKGLECSTLYRTQSSSNPAELRQLLDCDAASVDLEMIDVHVLADAFKRYLADLPNPVIPVAVYNEMMSLAQELQSPEDCIQLLKKLIRLPNIPHQCWLTLQYLLKHFFKLSQASSKNLLNARVLSEIFSPVLFRFPAASSDNTEHLIKAIEILISTEWNERQPAPALPPKPPKPTTVANNSMNNNMSLQDAEWYWGDISREEVNEKLRDTADGTFLVRDASKMHGDYTLTPRKGGNNKLIKIFHRDGKYGFSDPLTFNSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNHDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 35 - DNA, 3446 bp, Mus musculus
ggaaggatgaggcgcccgcggcggcccgggggctccgggggctccgggggctccgggggcctccggctgctggtctgcctgctgttgctgagcggccgccccgggggctgcagcgccatcagtgcccacggctgtctgtttgaccgcagactttgttcgcatctggaagtctgtattcaggatggcttgtttggacagtgccaggcaggagtggggcaggcacggcccctcttacaagtcacttccccagttctccagcgcctacaaggtgtgctccggcaactcatgtcccaaggcttgtcctggcatgatgaccttacccagcatgtgatctcccaggagatggaacgcatccccaggcttcgccccccagagccccatccaagggacaggtctggtttggtgcccaggaaaccaggccctgcaggggaattgctaactcagggcaatcctactggctcctctcctgctgcccagggctttccaaggcctgcagggggacggagctggggcggctccccactgtcctctctgcaggctgagttgttaccccctctcttggagcatctgctaatgcccccacagcctccacaccctgctctgacctatgaacctgcactgctacagccttacctcttccaccagtttggctcccgagatggctcccggggctcagagagctcctctggggtagttggtgttggtcacctgtccaaggctgaaggtcctgcactcttcagcagaagtgcctccaaggccattttggggactcactctggacactcttttggggaccttacaggtccctcacctgctcaacttttccaagattcagggctgctctacatggcccaagagttgccagtgcctggcagagcccgggcaccaaggttgccagagaatgggggcaacagggcagaggactcttcagagggccatgaggaggaagtactagggggtcgtggggagaagtcccctccccaagcagcacaaccagaattgagtctgcagagattgactgctgtactggcaggctatggagtagagctgcgtcagttgaccccggagcagttttctaccctcttgaccctgatgcagttgctgcccaagggcacaggaagaaatcttgaaggggctgtaaatgttggaggagccgatgtcaagaaaacaatacaacagatgcagagaggagacccagcagaagctctgccccccacaccctcgcttcctgggtacctcactgccagccctgcctccagcgaagttcagcaggtgctgagccctggtttccctgaacctccccacacacccagccctctgggctcctcctcagtccttctggagaagaaaagtcccttgggccagagccagcccacagtggtgggacggccatcagctcgaccatcggccgaggagtatggctatatcgtcactgaccagaaacccctgagcctggtggctggagtgaggctgctggagattctggctgagcacgtgcatatgtcctccggtagctttatcaacatcagtgtggtgggaccagctgtcaccttccgaatccggcacaatgagcagaacctgtctttggcagatgtgacccagcaagctgggctggtgaagtctgaactggaagcgcagacagggctccagattttgcagacaggggtgggacagagggaggaagcagctgaagtccttccccgacaagcccatggcatatctcccatgcgctcagtgctgcttactctagtggccctggcaggcgtcgctgggctgctagtggctttggcagtggccttgtgtatgcgccatcattcgagacagcgggataaggagcgcctggcagcgctggggccggagggggcccatggtgacactacttttgagtaccaggacctgtgtcgccagcacatggccacaaagtccctgtttaaccgggcggagggtcagccagagccttctagggtgagcagtgtgtcctcccagttcagcgacgcggcccaggccagccccagttcccacagcagctctccatcttggtgcgaggagcccgcccaggccaacatggacatctccacaggacacatgattctggcatacatggaggatcaccttcggaaccgggaccggttggccaaggagtggcaggctctgtgcgcctaccaagcagagccaaacacctgtgccgccgcacaggatgagagcaacatcaagaagaaccgccatcctgacttcctaccctatgaccatgcccgaatcaagctgaaagtggagagcagcccttctcggagtgattacatcaacgccagccccatcatcgagcatgaccctcggatgccggcctacatagccacacagggaccactgtcccacaccatcgcggacttctggcagatggtgtgggagagtggctgcactgtcatcgttatgctgaccccgttggtggaggacggtgtcaaacagtgtgaccgctactggccggatgaaggatcttccctctaccacgtctatgaggtgaacctggtgtcggagcacatctggtgcgaggacttcctggtgcggagcttctaccttaagaacctgcagacccaggagacgcgcacgctcactcagttccacttcctcagctggccggcagagggcactccggcctccacccggccgctgctggacttccgcaggaaagtgaacaagtgctacagaggccgctcctgccccatcatagtgcactgcagtgacggtgcagggaggacaggcacctacatccttattgacatggtcctgaatcgcatggccaaaggagtgaaggagattgatattgctgccaccctggagcatgtccgtgaccagcggcctggacttgtccgttctaaggaccagtttgagtttgcgctgacagccgtggcagaggaggtgaatgctatcctcaaggccctgccccagtgagcccccctgggcgcctcagtgggcatcctggcctcggctccttctgcctgtgtgagcatctgtgcacccactcttcagcccctacccatctgccaccttggtctgacttggccatgggagcctttcccaacccagtgtggaagggagtcgggagggaaggaaggggtaggctcgccctgctttatccatgctagaaccatggtatcccatgggaagcagacagcaggcaaggagaggcgtggacaccggccacaggtgtgcccgagccccatcctacctgagtctctgtctccctctctggatatgtgcgtccccactcccaccagcctaccacctatagacaaagcagaacgaggaaaccccagctcccccaaccctgctaccactggcctgccaccttgaccctgctcaaccttctccctctagcacaagggaacatttctagaaaagtaaaatctacttttgtatcagtgtgaataaagttagtgtgttgtctgtgc

SEQ ID NO: 36 - protein, 978 aa, Mus musculus
MRRPRRPGGSGGSGGSGGLRLLVCLLLLSGRPGGCSAISAHGCLFDRRLCSHLEVCIQDGLFGQCQAGVGQARPLLQVTSPVLQRLQGVLRQLMSQGLSWHDDLTQHVISQEMERIPRLRPPEPHPRDRSGLVPRKPGPAGELLTQGNPTGSSPAAQGFPRPAGGRSWGGSPLSSLQAELLPPLLEHLLMPPQPPHPALTYEPALLQPYLFHQFGSRDGSRGSESSSGVVGVGHLSKAEGPALFSRSASKAILGTHSGHSFGDLTGPSPAQLFQDSGLLYMAQELPVPGRARAPRLPENGGNRAEDSSEGHEEEVLGGRGEKSPPQAAQPELSLQRLTAVLAGYGVELRQLTPEQFSTLLTLMQLLPKGTGRNLEGAVNVGGADVKKTIQQMQRGDPAEALPPTPSLPGYLTASPASSEVQQVLSPGFPEPPHTPSPLGSSSVLLEKKSPLGQSQPTVVGRPSARPSAEEYGYIVTDQKPLSLVAGVRLLEILAEHVHMSSGSFINISVVGPAVTFRIRHNEQNLSLADVTQQAGLVKSELEAQTGLQILQTGVGQREEAAEVLPRQAHGISPMRSVLLTLVALAGVAGLLVALAVALCMRHHSRQRDKERLAALGPEGAHGDTTFEYQDLCRQHMATKSLFNRAEGQPEPSRVSSVSSQFSDAAQASPSSHSSSPSWCEEPAQANMDISTGHMILAYMEDHLRNRDRLAKEWQALCAYQAEPNTCAAAQDESNIKKNRHPDFLPYDHARIKLKVESSPSRSDYINASPIIEHDPRMPAYIATQGPLSHTIADFWQMVWESGCTVIVMLTPLVEDGVKQCDRYWPDEGSSLYHVYEVNLVSEHIWCEDFLVRSFYLNLQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 37 - DNA, 1933 bp, Mus musculus
cggcgggaggaaagcgcggtgaagccagattaggagcagcgagcacttggggacttagggccacaggacaccgcacaagatcgaccgactttttctggagaaccgcagaacgggcacgctggggtcgctggggctggccatggtgatggaggtgggcatcctggacgccggggggctgcgcgcgctgctgcgagagggcgccgcgcagtgcctgttgttggattgtcgctccttcttcgctttcaacgccggccacatcgcgggctcagtgaacgtgcgcttcagcaccatcgtgcggcgccgcgccaagggcgccatgggcctggagcatatcgtgcccaacgctgaactgcgtggccgcctgctggccggagcctaccacgccgtggtgctgctggacgagcgcagcgcctccctggacggcgccaagcgcgacggcaccctggccctggccgcgggcgcgctctgccgagaggcgcgctccactcaagtcttctttctccaaggaggatatgaagcgttttcggcttcctgccctgagctgtgcagcaaacagtccacccccacggggctcagcctccccctgagtactagtgtgcctgacagtgcagaatccggatgcagctcctgtagtacccctctctacgatcaggggggcccagtggagatcctgtccttcctgtacctgggcagtgcctatcacgcttctcggaaggatatgcttgacgccttgggcatcaccgccttgatcaacgtctcagccaattgtcctaaccactttgagggtcactaccagtacaagagcatccctgtggaggacaaccacaaggcagacatcagctcctggttcaacgaggctattgacttcatagactccatcaaggatgctggagggagagtgtttgttcattgccaggccggcatctcccggtcagccaccatctgccttgcttacctcatgaggactaaccgggtaaagctggacgaggcctttgagtttgtgaagcagaggcggagtatcatctccccgaacttcagcttcatgggccagctgctgcagtttgagtcccaagtgctagcccctcactgctctgctgaagctgggagccctgccatggctgtccttgaccggggcacctctactaccacagtcttcaacttccctgtttccatccccgtccaccccacgaacagtgccctgaactaccttaaaagccccatcaccacctctccaagctgctgaagggcaaggggaggtgtggagtttcacttgccaccgggtcgccactcctcctgtgggaggagcaatgcaataactctgggagaggctcatgggagctggtccttatttatttaacacccccctcaccccccaactcctcctgagttccactgagttcctaagcagtcacaacaatgacttgaccgcaagacatttgctgaactcggcacattcgggaccaatatattgtgggtacatcaagtccctctgacaaaacagggcagaagagaaaggactctgtttgaggcagtttcttcgcttgcctgttttttttttctagaaacttcatgcttgacacacccaccagtattaaccattcccgatgacatgcgcgtatgagagtttttacctttatttatttttgtgtaggtcggtggtttctgccttcacaaatgtcattgtctactcatagaagaaccaaatacctcaattttgtgtttgcgtactgtactatcttgtaaatagacccagagcaggtttgctttcggcactgacagacaaagccagtgtaggtttgtagctttcagttatcgacagttgtatgtttgtttatttatgatctgaagtaatatatttcttcttctgtgaagacattttgttactgggatgactttttttatacaacagaataaattatgacgtttctattga

SEQ ID NO: 38 - protein, 367 aa, Mus musculus
MVMEVGILDAGGLRALLREGAAQCLLLDCRSFFAFNAGHIAGSVNVRFSTIVRRRAKGAMGLEHIVPNAELRGRLLAGAYHAVVLLDERSASLDGAKRDGTLALAAGALCREARSTQVFFLQGGYEAFSASCPELCSKQSTPTGLSLPLSTSVPDSAESGCSSCSTPLYDQGGPVEILSFLYLGSAYHASRKDMLDALGITALINVSANCPNHFEGHYQYKSIPVEDNHKADISSWFNEAIDFIDSIKDAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHPTNSALNYLKSPITTSPSC

SEQ ID NO: 39 - DNA, 1981 bp, Mus musculus
atacccatcaaatgaagcaccaaagacaactgtgttgagcgcccctgggcctggctcattcttaatcatattgccatctttactgctctgattcctaggatccccgcaatgactctgaataatgtcaccatgcgccaaggcactgtgggcatgcagccacagcagcgctggagtatgcctgctgatgccaggcatctgatggtccagaaggatccccacccctgcaacctccgaaaccgccactctactgctccggaagagcactgccggcggacgtggtcgtccgactccacagactcggtcatctcttctgagtctggaaacacctactaccgagtagtgcttataggggagcaaggagtgggcaagtccaccctggccaacatctttgcaggtgtgcatgacagcatggacagcgactgtgaggtcttgggagaagatacatatgagcgtaccctggtcgttgacggagagagtgcaaccattatcctcctggacatgtgggaaaataagggggagaacgaatggctccatgaccactgcatgcaggtcggggatgcctatctgatcgtctactctatcacagaccgtgcaagcttcgagaaggcatctgagctgaggattcagctccgcagggcccggcagacagaagacattcctataattttggttggcaacaaaagcgacttagtgcggtgtcgagaagtgtctgtgtcagaagggagagcttgtgctgtggtgttcgactgcaaattcatcgagacctctgcggctgtgcagcacaacgtgaaggaactgtttgagggcattgagcgacaggtgcgcctgccgagggacagcaaggagaagaatgagaggaggctggcctaccagaagaggcgggagagcattcccaggaaagccaggcgcttctggggcaaaattgtggccaaaaacaacaagaacatggcttcaagctccaagtcaaaatcctgccatgacctgtctgtgctctaggcacccagtgtcacccagatgtcccttggtggacatcgttgaaggctattgggaccagtgatctatattagattggatacataagcattgttagacgcaacttcccctatggcagatggaaaccaacaggttagccttgtgggcaaccaagtgcacgggcaatgaatgagctctgtcaagagtcagtatttattcataggaaaagcttgagctgctacgtggatgtctcagactcatttaagacacgcttcgggttcacatgagtttctctctcctttggacagcagaatatttttttcctcagtgttgttccatgtgatttcgaggtccttgggtcatatagaaatgtagggaaatggcggtagtttattggaaggagaagggctcactgcatacttatatccctgaaacgacatctttagagctggcctcatcacagttgtgactatttctgctccagtgaagagaattgttagatttgctggaaactgaggcttactaacagtttttgtttaaagaccacagagattgtagacttaggagctatatggtactacttataggttcaaaaaattgtttacttatgtgtcgtagaagtgtttattttgaggaaactatttttttttttgccaaattctacttagtcaaatcatcttctatgtcttgctgttttttaaatcattaagctatcataaaatattttttaaaaaaatctcaactatattgataacctgcagtgcaaaattttaaatatagtcctgtttttcccccaaaacataaacatgccccatcctttgggttgcttctgtatgccacagctgaattatatttattattttgcaataaccattttatatttgataaagatatttatgagcatatttcttactgagaaaatgtctgttttattacctttttatatttttcaaagtattcaagtttttacctattgtcttataataaataaataaaatctttgaaaagg

SEQ ID NO: 40 - protein, 295 aa, Mus musculus
MTLNNVTMRQGTVGMQPQQRWSMPADARHLMVQKDPHPCNLRNRHSTAPEEHCRRTWSSDSTDSVISSESGNTYYRVVLIGEQGVGKSTLANIFAGVHDSMDSDCEVLGEDTYERTLVVDGESATIILLDMWENKGENEWLHDHCMQVGDAYLIVYSITDRASFEKASELRIQLRRARQTEDIPIILVGNKSDLVRCREVSVSEGRACAVVFDCKFIETSAAVQHNVKELFEGIERQVRLPRDSKEKNERRLAYQKRRESIPRKARRFWGKIVAKNNKNMASSSKSKSCHDLSVL

SEQ ID NO: 41 - DNA, 1242 bp, Mus musculus
tatcccgctgttgctgcaagccggctgcatcttagttggccatgaagaccccagcacagcggctgcaccttcttccactgttgttgctgctttgtggtgagtgtgcccaggtatgcggctgcaacgagacagggatgctggagaggctgcctcgctgtgggaaagccttcgctgacatgatgcagaaggtggctgtctggaagtggtgcaacctgtcggagttcatcgtgtattatgaaagcttcactaactgcaccgagatggagaccaacatcatgggctgctactggcccaacccgctggcccagagcttcatcactggaatccacaggcagttcttttccaactgcacggtggacaggacccactgggaagaccccccggatgaagtactcatcccactgatcgcggttcctgtcgtgctgactgtggctatggctggcctggtggtgtggcgcagcaagcacactgatcggctgctgtgaggatctgctggatggagggccatgcctggcaggctgggagaatgttgctcagagctctgagagctggcagactcggcttctgtctggtttgctttggccacaccctacctggccatgccaaagtcctcccgaccaggctggtgtggcccttgctgtctagcctgccgcctgctggggttcagattgtccatactttgctctttcttgggctagtggaagaaagtgacaaatcccaagtttgtggaccaggcatggaaatcaactgttgctgagccccgctccccaggctcggttccctagtttctagccgtttcttggcagagtcttgctcagcctgaaccccgccccaggtcctgacccatttctagtcctgaccctgacccctgctacacttggccagagagggcaggcaaggtcatctggaagatgtggacgcccccccgcctctattcaagagactgagcacatcatttatcagacatgaaggatagcctggggtcattaggagccacgtgtgacctactgacccacctgcctgtcctctctgtgatctgtcacgattctgtgtccagtgtgggctggagctgtggcttgtttagcccttcaaagacacctaccctgcaggtagagcgtgaacctccttcttgaggggtattcctgggagtggggcgcactgagtgtgctcaagggttctgtctgctgatgtcagttctttttgattaaagtgtctccttacaaaaaaaaaaaaaaaaaaaaa

SEQ ID NO: 42 - protein, 147 aa, Mus musculus
MKTPAQRLHLLPLLLLLCGECAQVCGCNETGMLERLPRCGKAFADMMQKVAVWKWCNLSEFIVYYESFTNCTEMETNIMGCYWPNPLAQSFITGIHRQFFSNCTVDRTHWEDPPDEVLIPLIAVPVVLTVAMAGLVVWRSKHTDRLL

SEQ ID NO: 43 - DNA, 1115 bp, Mus musculus
atgctcaacaaagccaagaattcaaagagtgcccagggtctggctggtcttcgaaaccttgggaacacgtgcttcatgaactcaattcttcagtgcctgagcaacacccgagagctgagagattactgcctccagaggctgtacatgcgggacctcggccacaccagcagcgctcacacggccctcatggaagagtttgcaaaactaatccagaccatatggacgtcgtcccccaatgatgtggtgagcccatctgagttcaagacccagatccagagatatgcgccacgcttcatgggctataatcagcaggatgctcaggaattccttcgtttccttctggatggtctccacaatgaggtgaaccgggtggcagcaaggcctaaggccagccctgagacccttgatcatctccctgatgaagaaaaggggcgacagatgtggaggaagtatctggaaagggaagacagtcggattggggatctcttcgttgggcagctgaagagctccctcacatgcaccgattgtggctactgctctacagtcttcgatcccttctgggatctctcgttgcccatcgcaaagagaggttaccctgaggtgacgttaatggattgtatgaggctcttcaccaaagaggacatattggatggtgatgagaagccaacttgctgccgctgccgagccagaaaacgatgcataaaaaagttctctgtccagaggttcccaaagatcttggtgctccacctgaagcgattctcagaatccaggatacgaaccagcaagctcacaacatttgtgaatttcccactaagagacctggacttgagagaatttgcttcagaaaacaccaaccatgctgtttacaacctgtatgctgtgtccaatcactccggaaccaccatgggaggccactatacagcctactgccgaagtccggttacaggcgaatggcacactttcaatgattccagtgtcacacccatgtcctccagccaagtgcgcaccagcgacgcctatttgctcttctatgaactggccagtccaccctcccgtatgtagcattgaggagctgcggcccttccctcttccctgtggtggccccacgtcctaag

SEQ ID NO: 44 - protein, 353 aa, Mus musculus
MLNKAKNSKSAQGLAGLRNLGNTCFMNSILQCLSNTRELRDYCLQRLYMRDLGHTSSAHTALMEEFAKLIQTIWTSSPNDVVSPSEFKTQIQRYAPRFMGYNQQDAQEFLRFLLDGLHNEVNRVAARPKASPETLDHLPDEEKGRQMWRKYLEREDSRIGDLFVGQLKSSLTCTDCGYCSTVFDPFWDLSLPIAKRGYPEVTLMDCMRLFTKEDILDGDEKPTCCRCRARKRCIKKFSVQRFPKILVLHLKRFSESRIRTSKLTTFVNFPLRDLDLREFASENTNHAVYNLYAVSNHSGTTMGGHYTAYCRSPVTGEWHTFNDSSVTPMSSSQVRTSDAYLLFYELASPPSRM

SEQ ID NO: 45 - DNA, 3034 bp, Mus musculus
gcggagcgtgagctgtgcgagcgagcgagcgcgagcatagcctgcgagcgagcagagagaaagagcgagggcaagagagcggcgaggcgcctgcgcgatgctcgggcccctaagcccgcggcgctgagccagccgggacggacatgcgcgggagggcgccgcggggtcccgctcccttgggggaatgaaagctactggttgacttaaaaacacctgggctttacaaatttgaaggcatcccagagtggggcacaatgtcaacagcaggagttgctgctcaggatattcgagtcccattaaaaactggatttctccataatggtcaggccttggggaatatgaagtcctgctggggcagtcacagtgagtttgaaaataactttttaaatattgatccaataaccatggcctacaatctgaactcccctgctcaggagcacctaacaactgttggatgtgctgctcggtctgctccagggagcggccacttctttgcagagtgtggtccatctccaaggtcaagcttgccccctcttgttatctcaccaagtgaaagctcgggacagcgtgaagaggatcaagttatgtgtggttttaagaaactctcagtgaatggggtctgcacttccacacctccacttacacccattaaaagctgcccttcccctttcccctgtgcggctctgtgtgatcggggttctcggccgctcccgccactgcccatctctgaagacctatgtgtggatgaggccgacagtgaggtagagcttctaaccaccagctcagacacagacttgcttttagaagactctgcgccttcagatttcaaatacgatgctcctggcaggcgcagcttccgtgggtgcggccagatcaactatgcatattttgacagcccaactgtttctgtggcagatcttagctgtgcatctgaccagaacagagttgttccagacccaaaccctcccccacctcaaagccatcgcagattaaggaggtctcactcaggaccagctgggtcatttaacaagccagccattcggatatctagctgcacacacagagcttctcctagctctgatgaagacaagcctgaggtccctcccagggttcctatacctcctaggccagcaaagccagactatagacggtggtcagcagaagtgacctccaacacctacagtgatgaagataggcctcccaaagtccccccgagagaacctttgtctcggagtaactcccgtaccccaagtcctaaaagccttccgtcttacctcaatggggtcatgcccccaacacagagcttcgctcctgaccccaagtatgtcagcagcaaagccctgcagagacagagcagcgaaggatctgccaacaaggttccttgcatcctgcccattattgaaaatgggaagaaggttagctcaacgcattattacttactacctgagaggccaccgtacctggacaaatatgaaaagtattttaaggaagcagaagaaacaaacccaagcacccaaattcagccattacctgctgcctgtggtatggcctctgccacagaaaagctggcctccagaatgaaaatagatatgggtagccacgggaagcgcaaacacttatcctacgtggtttctccataaatatgggggtcatgattcaacagaagttacatgggatgaatggctcccagttttccagtttgaggttcgtagaacaatgtcaagtggcaaaatgaagttggtggactccgccttaatgagaaaggcttagagcagttatgaggtgctgttatgctgggagtccctgatctatcagcataggagaaaaaagtatgatttaaagatgtgctagggggagggaaaaatgggcaacttttacatttgactacattatatacctatgtataaaagtgcggtgtaaccatagaccatagctgcaggataaccaattagtcactcttagagtaatctgtattcagaacaattcaaacaagctggaggaacagctcctgatagtgtgagaattgagcaaatgggagaaagcaatattgttagatcagattataaatttgttaagtttaaagattcctggcatacaggcctgctctataaatttgttttccccttccctgccagcagtcttctccatacacgacagggcgtgttctccaccaggcctgtaacatcttgttgagatcatttctatggcccaatacttgtcgctctggggttttgtcttgttgaggagaggacagcagtttctggaccatgttatcacctgtgtgtgtctcatatcttggaaattgacagatttggtgaataacttttccatactattcctgcttttcccatccactgaaacagcctgttgtagcaagaggctttcaagagtgcagtggagttgcgctggccatcagtgtttggggtctgagtttgatagactagtgcagcgatcagccatatgattgagagctactttggggatatatggtacgttgtttttgttttttagacttaataaaggacaacacgagctggtcttgtgttgctggttcctattcagtatttcctggggattgtttgctttttaagtgaaacacttctgaccaatagcacagaacgtcttaatgccagaggtcacttcagcatcttcctgctttgaaaactcacgctggctgcttcactgccctgagattcagtgagacacgcagtttgtgttcagtttttacatcctctgattgtttatcttgtgcagataaacacaaagagaaggtgcttgctagcagggacactgctgccatgtcccaacaagctgttcagtttaaactgctgaatgacattatttgagctatttaaagcttactttagtatgaactaaatgaaggttaaaacatgctttagaaaaatgcactgatctccgcactgtgtgtacagtattggacaaaggatttattcattttgttgcattattttgaatattgtcttttcattttaataaagttatattacttatttatgaaaaaaaaaaaaaaaaa

SEQ ID NO: 46 - protein, 461 aa, Mus musculus
MSTAGVAAQDIRVPLKTGFLHNGQALGNMKSCWGSHSEFENNFLNIDPITMAYNLNSPAQEHLTTVGCAARSAPGSGHFFAECGPSPRSSLPPLVISPSESSGQREEDQVMCGFKKLSVNGVCTSTPPLTPIKSCPSPFPCAALCDRGSRPLPPLPISEDLCVDEADSEVELLTTSSDTDLLLEDSAPSDFKYDAPGRRSFRGCGQINYAYFDSPTVSVADLSCASDQNRVVPDPNPPPPQSHRRLRRSHSGPAGSFNKPAIRISSCTHRASPSSDEDKPEVPPRVPIPPRPAKPDYRRWSAEVTSNTYSDEDRPPKVPPREPLSRSNSRTPSPKSLPSYLNGVMPPTQSFAPDPKYVSSKALQRQSSEGSANKVPCILPIIENGKKVSSTHYYLLPERPPYLDKYEKYFKEAEETNPSTQIQPLPAACGMASATEKLASRMKIDMGSHGKRKHLSYVVSP

SEQ ID NO: 47 - DNA, 2328 bp, Mus musculus
atggctgaacaacttcttcctcaggctttgtatttgagcaatatgcggaaagctgtgaagatacgagagagaaccccagaagacattttcaaacctaccaatgggatcatctatcactttaaaaccatgcaccgatacacgctggagatgttcagaacatgccagttttgcccacagttccgagagatcatccacaaagcacttattgacagaagtgtccaggcttccctggaaagccagaagaagctcaactggtgtcgtgaagtcaggaagctcgtggctctgaaaaccaatggtgatggaaactgcctcatgcatgcagcttgtcagtacatgtggggtgttcaggatactgacctggtcctgaggaaggccctctgcagcacccttaaggagacagacactcggaactttaaattccgctggcagctggaatctctgaaatctcaggaatttgtggaaacaggactttgctacgacactcggaactggaatgacgaatgggacaacttggtcaaaatggcatcagcagacacacctgcagcccgaagtggacttcagtacaattccctggaagaaatccacatatttgtcctcagcaacatcctcagaagacccatcattgtcatttcagacaaaatgctaagaagtttggaatctggttccaattttgctcctttgaaagtgggtgggatttatctgcctcttcactggcctgcccaggagtgttacagatatcccatcgtcctaggctatgacagccagcactttgtacccctggtgaccctgaaggacagtggacctgaacttcgcgctgttccacttgttaacagagaccggggtaggtttgaagacttaaaagttcacttcttgacagatcctgagaatgagatgaaggaaaagcttctaaaggagtacttgatagtgatggagatccctgtgcaaggctgggaccacggcacgactcacctgatcaacgctgcaaaattggatgaagctaacttacccaaagaaataaatttggtagacgattactttgagcttgttcagcacgaatacaagaaatggcaggagaacagcgatcaggccaggagagcggcacatgcgcagaaccccttggagccttccacaccccagctatcactcatggatataaaatgtgagacacccaactgtcctttcttcatgtccgtgaacactcagcctttatgccacgaatgctcagagaggcgccaaaagaatcagagcaagctcccaaagctgaactcgaagctaggccctgaaggactcccaggcgtgggacttggctcctcaaactggagccccgaggaaaccgctggaggacctcattcagccccacccacagcacccagcctttttctcttcagtgagaccactgcaatgaagtgcaggagtcctgggtgcccttttactttgaatgtgcagcataatggattctgtgagcgttgccacgcccggcagattaatgccagccacaccgcagaccctggaaagtgccaagcctgccttcaggatgtcactcggacctttaatggcatctgcagtacctgtttcaaaaggactacagcagagcccagctccagcctcacttccagtatccctgcctcctgtcaccaacgctccaagtctgacccctcacaactcatccaaagtctcactccacactcttgccaccggactggaaatgtctctccttctggctgcctctcccaggctgcacggactccaggagacagagcagggacaagcaagtgcaggaaagctggctgcatgtattttgggactccagaaaacaagggcttttgcactctatgtttcatcgaatacagagaaaataagcagtctgttactgcctctgcgaaagctggttccccggcccccaggttccagaacaatgtcccgtgcctgggcagggagtgcggcacactcggaagcaccatgtttgaagggtactgtcagaagtgtttcatcgaagctcagaaccagagattccatgaagcaagaagaacggaagaacagctgagatcaagccagcatagagacatgcctcgaactacacaggtagcctcaaggctgaaatgtgcccgggcctcctgcaagaacattctggcctgtcgcagtgaggaactctgtatggagtgccagcacctaagccaacgagtaggttctgtggcccaccggggtgagcccacgcctgaagagccccctaaacagcgctgccgggcccctgcttgtgatcactttggcaatgccaagtgtaatggttactgcaatgagtgctaccagttcaagcagatgtatggctaa

SEQ ID NO: 48 - protein, 775 aa, Mus musculus
MAEQLLPQALYLSNMRKAVKIRERTPEDIFKPTNGIIYHFKTMHRYTLEMFRTCQFCPQFREIIHKALIDRSVQASLESQKKLNWCREVRKLVALKTNGDGNCLMHAACQYMWGVQDTDLVLRKALCSTLKETDTRNFKFRWQLESLKSQEFVETGLCYDTRNWNDEWDNLVKMASADTPAARSGLQYNSLEEIHIFVLSNILRRPIIVISDKMLRSLESGSNFAPLKVGGIYLPLHWPAQECYRYPIVLGYDSQHFVPLVTLKDSGPELRAVPLVNRDRGRFEDLKVHFLTDPENEMKEKLLKEYLIVMEIPVQGWDHGTTHLINAAKLDEANLPKEINLVDDYFELVQHEYKKWQENSDQARRAAHAQNPLEPSTPQLSLMDIKCETPNCPFFMSVNTQPLCHECSERRQKNQSKLPKLNSKLGPEGLPGVGLGSSNWSPEETAGGPHSAPPTAPSLFLFSETTAMKCRSPGCPFTLNVQHNGFCERCHARQINASHTADPGKCQACLQDVTRTFNGICSTCFKRTTAEPSSSLTSSIPASCHQRSKSDPSQLIQSLTPHSCHRTGNVSPSGCLSQAARTPGDRAGTSKCRKAGCMYFGTPENKGFCTLCFIEYRENKQSVTASAKAGSPAPRFQNNVPCLGRECGTLGSTMFEGYCQKCFIEAQNQRFHEARRTEEQLRSSQHRDMPRTTQVASRLKCARASCKNILACRSEELCMECQHLSQRVGSVAHRGEPTPEEPPKQRCRAPACDHFGNAKCNGYCNECYQFKQMYG

SEQ ID NO: 49 - DNA, 499 bp, Mus musculus
  modified_base (461): a, g, c or t
ttttttttcatacttgataaaattttatttaaaaaaaaagagaataataatttatacccttgacaaaataaaagatcttataatataaatgtttcttagaaaatatatgaaaagataatattacaaatattaataaatcaatattcacatgacagcaaaagtggcaatgattctacaagaaggtgaggaggaagatgctttccggtccgcagcaatgtctctggagaggcctcctgtcccttctttctccttcaatgaggtgtgctcctattttaagaaaacctgatacaagcagatctaatcagtttaggaagctggtatttatttgcaccgcaaaataatttttttacaaaaaaaattctatcaaggatcctttaaatatcaagtttcccaatgcacttagaatacagttaaccaaatttacaagtcttcgacttctctctggtgtagctctaccgcanggcgtgaggtattgctgaagtgagtgcgtgcgtccgtg

SEQ ID NO: 50 - DNA, 21 bp, Artificial Sequence
  Description of Artificial Sequence Synthetic oligonucleotide
catcttggcctcactgtccac

SEQ ID NO: 51 - DNA, 25 bp, Artificial Sequence
  Description of Artificial Sequence Synthetic oligonucleotide
tgcttgctgatccacatctgctgga

SEQ ID NO: 52 - DNA, 19 bp, Artificial Sequence
  Description of Artificial Sequence Synthetic oligonucleotide
gggccggactcatcgtact

SEQ ID NO: 53 - DNA, 21 bp, Artificial Sequence
  Description of Artificial Sequence Synthetic oligonucleotide
gggaggaccttacctgttcgt

SEQ ID NO: 54 - DNA, 22 bp, Artificial Sequence
  Description of Artificial Sequence Synthetic oligonucleotide
caccaggctgtgggcctcaagg

SEQ ID NO: 55 - DNA, 20 bp, Artificial Sequence
  Description of Artificial Sequence Synthetic oligonucleotide
ccagatgtggatgcttgcaa

SEQ ID NO: 56 - DNA, 19 bp, Artificial Sequence
  Description of Artificial Sequence Synthetic oligonucleotide
gggagaacagaagcgcctg

SEQ ID NO: 57 - DNA, 29 bp, Artificial Sequence
  Description of Artificial Sequence Synthetic oligonucleotide
agaagggtgaggatcccccaaatcagagt

SEQ ID NO: 58 - DNA, 21 bp, Artificial Sequence
  Description of Artificial Sequence Synthetic oligonucleotide
cccttgtttcaatcactccca

SEQ ID NO: 59 - DNA, 21 bp, Artificial Sequence
  Description of Artificial Sequence Synthetic oligonucleotide
tttctgaacagtgaggtccgc

SEQ ID NO: 60 - DNA, 19 bp, Artificial Sequence
  Description of Artificial Sequence Synthetic oligonucleotide
ccggaagaggtggcggcga

SEQ ID NO: 61 - DNA, 20 bp, Artificial Sequence
  Description of Artificial Sequence Synthetic oligonucleotide
gggctctgatggagtgcttg

SEQ ID NO: 62 - DNA, 21 bp, Artificial Sequence
  Description of Artificial Sequence Synthetic oligonucleotide
ttggaagacccgtttgagcta

SEQ ID NO: 63 - DNA, 26 bp, Artificial Sequence
  Description of Artificial Sequence Synthetic oligonucleotide
tcttgcagaattcctctgctcctggg

SEQ ID NO: 64 - DNA, 20 bp, Artificial Sequence
  Description of Artificial Sequence Synthetic oligonucleotide
tttcttgccagttttgggct

SEQ ID NO: 65 - DNA, 23 bp, Artificial Sequence
  Description of Artificial Sequence Synthetic oligonucleotide
agacttccatgggaatgatagca

SEQ ID NO: 66 - DNA, 20 bp, Artificial Sequence
  Description of Artificial Sequence Synthetic oligonucleotide
cctctcgtccggcagctggc

SEQ ID NO: 67 - DNA, 20 bp, Artificial Sequence
  Description of Artificial Sequence Synthetic oligonucleotide
aatgtgtaagccgggcagaa

SEQ ID NO: 68 - DNA, 27 bp, Artificial Sequence
  Description of Artificial Sequence Synthetic oligonucleotide
aatcctttgtccaatactgtacacaca

SEQ ID NO: 69 - DNA, 22 bp, Artificial Sequence
  Description of Artificial Sequence Synthetic oligonucleotide
gaaaaatgcactgatctccgca

SEQ ID NO: 70 - DNA, 30 bp, Artificial Sequence
  Description of Artificial Sequence Synthetic oligonucleotide
gtatgaactaaatgaaggttaaaacatgct

SEQ ID NO: 71 - DNA, 22 bp, Artificial Sequence
  Description of Artificial Sequence Synthetic oligonucleotide
ccatggtgcttgttaacgcttt

SEQ ID NO: 72 - DNA, 26 bp, Artificial Sequence
  Description of Artificial Sequence Synthetic oligonucleotide
cccaacttgtagctggtaaagcttca

SEQ ID NO: 73 - DNA, 23 bp, Artificial Sequence
  Description of Artificial Sequence Synthetic oligonucleotide
cctgtctaccttctggtctccaa

SEQ ID NO: 74 - DNA, 23 bp, Artificial Sequence
  Description of Artificial Sequence Synthetic oligonucleotide
cttgctgatcgtctggagagttt

SEQ ID NO: 75 - DNA, 22 bp, Artificial Sequence
  Description of Artificial Sequence Synthetic oligonucleotide
tgctgatgaccctccattgtga

SEQ ID NO: 76 - DNA, 24 bp, Artificial Sequence
  Description of Artificial Sequence Synthetic oligonucleotide
ttaacattcctgaggttgcaagaa

SEQ ID NO: 77 - DNA, 19 bp, Artificial Sequence
  Description of Artificial Sequence Synthetic oligonucleotide
gccgtgtgtatggctgcat

SEQ ID NO: 78 - DNA, 20 bp, Artificial Sequence
  Description of Artificial Sequence Synthetic oligonucleotide
cagcccccaggccactgtgg

SEQ ID NO: 79 - DNA, 23 bp, Artificial Sequence
  Description of Artificial Sequence Synthetic oligonucleotide
aggagggataacaggtgctgtgt

SEQ ID NO: 80 - DNA, 19 bp, Artificial Sequence
  Description of Artificial Sequence Synthetic oligonucleotide
cctggaccccagccataga

SEQ ID NO: 81 - DNA, 24 bp, Artificial Sequence
  Description of Artificial Sequence Synthetic oligonucleotide
agcccatgtagtcccggtcactta

SEQ ID NO: 82 - DNA, 14 bp, Artificial Sequence
  Description of Artificial Sequence Synthetic oligonucleotide
tgcgccggccaaaa

SEQ ID NO: 83 - DNA, 19 bp, Artificial Sequence
  Description of Artificial Sequence Synthetic oligonucleotide
gcttggacagccccagatc

SEQ ID NO: 84 - DNA, 24 bp, Artificial Sequence
  Description of Artificial Sequence Synthetic oligonucleotide
ggtactcagattcccacaccgctt

SEQ ID NO: 85 - DNA, 20 bp, Artificial Sequence
  Description of Artificial Sequence Synthetic oligonucleotide
tgtgtgtacgtgcccagcat

SEQ ID NO: 86 - DNA, 19 bp, Artificial Sequence
  Description of Artificial Sequence Synthetic oligonucleotide
gcgagtttgaaggcatgca

SEQ ID NO: 87 - DNA, 23 bp, Artificial Sequence
  Description of Artificial Sequence Synthetic oligonucleotide
ctctcttcacccgccgatgcgat

SEQ ID NO: 88 - DNA, 23 bp, Artificial Sequence
  Description of Artificial Sequence Synthetic oligonucleotide
ccatgtttctccaggtcaaaatg

SEQ ID NO: 89 - DNA, 19 bp, Artificial Sequence
  Description of Artificial Sequence Synthetic oligonucleotide
tggcagactcggcttctgt

SEQ ID NO: 90 - DNA, 25 bp, Artificial Sequence
  Description of Artificial Sequence Synthetic oligonucleotide
tttgctttggccacaccctacctgg

SEQ ID NO: 91 - DNA, 19 bp, Artificial Sequence
  Description of Artificial Sequence Synthetic oligonucleotide
ctggtcgggaggactttgg

SEQ ID NO: 92 - DNA, 18 bp, Artificial Sequence
  Description of Artificial Sequence Synthetic oligonucleotide
tggaccaatgccccagtt

SEQ ID NO: 93 - DNA, 28 bp, Artificial Sequence
  Description of Artificial Sequence Synthetic oligonucleotide
tcagtcaaagccgttggtgttttcattg

SEQ ID NO: 94 - DNA, 25 bp, Artificial Sequence
  Description of Artificial Sequence Synthetic oligonucleotide
gcccgttttataggtgacattttaa

(19) United States
(12) Patent Application Publication
(10) Pub. No.: US 2006/0252680 A1
(43) Pub. Date: Nov. 9, 2006

(54) Genes whose expression is increased in response to stimulation by corticotropin-releasing hormone

(76) Inventors: Pieter Johan Peeters, Dessel (BE); Hinrich Wilhelm Helmut Gohlmann, 's-Hertogenbosch (NL); Sigrid Maria Alice Swagemakers, Hulst (NL); Frederik Lucien Pieter Fierens, Deurne (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/532,740

(22) PCT Filed: Oct. 23, 2003
(86) PCT No.: PCT/EP03/11793

§ 371 Date: Apr. 26, 2005

(51) Int. Cl. A61K 38/35; A61K 48/00; C12Q 1/68; C12P 21/06

(52) U.S. Cl. 514/12; 514/44; 435/69.1; 435/6; 435/320.1; 435/325; 530/306

(57) ABSTRACT

The present invention relates generally to therapy and diagnosis of depression. In particular this invention relates to the polypeptides as well as to the polynucleotides encoding these polypeptides, wherein said polypeptides are shown to play a central role in mediating the endocrine response to corticotropin releasing hormone. These polypeptides and polynucleotides are useful in the diagnosis, treatment and/or prevention of depression.

The present invention relates generally to therapy and diagnosis of depression. In particular this invention relates to the polypeptides as well as to the polynucleotides encoding these polypeptides, wherein said polypeptides are shown to play a central role in mediating the cellular response to corticotropin releasing hormone. These polypeptides and polynucleotides are useful in the diagnosis, treatment and/or prevention of depression.

### BACKGROUND OF THE INVENTION

Recent socioeconomic analyses found that depression is a leading cause of disability and a major risk factor for development of other diseases. Moreover, on a world-wide scale depression is underdiagnosed and undertreated. Current antidepressant drugs have proven to be effective, but are burdened with slow onset of action and side effects. Above this, it is still unclear by which pharmacological mode of action they exert their clinical effects. Hypothesis-driven research based upon the corticosteroid receptor hypothesis of depression has led to a novel concept focusing on brain neuropeptide receptors, specifically the corticotropin-releasing hormone (CRH) receptor as drug target.

Corticotropin releasing hormone (CRH), a 41-amino acid polypeptide plays a central role in the regulation of the hypothalamic-pituitary-adrenal axis, mediating the endocrine responses to various stressors. Hypothalamic neurons release CRH into the hypophyseal portal system in response to stress, stimulating the secretion and biosynthesis of pituitary adrenocorticotropin (ACTH) leading to increased adrenal glucocorticoid production (1). Several clinical and preclinical studies point towards a causal role for alterations in the CRH system in the development of depression (2). The first studies with CRH in humans showed that the ACTH response to CRH is blunted in depressed patients, reflecting a CRH receptor desensitization secondary to continuously increased hypothalamic CRH secretion (3;4). In support of blunted ACTH response as consequence of increased CRH release is the finding of elevated CRH levels in cerebrospinal fluid of patients with depression. Other findings strengthening this notion of CRH hypersecretion in the depressed state are an increased number of CRH secreting neurons and a decreased number of CRH receptors in suicide victims who suffered from depression (5;6).

For CRH two high affinity receptors have been described, CRH-R1 and CRH-R2, both of which exist in several splice variant forms. Activation of these receptors by CRH results in G_{S}-mediated stimulation of adenyl cyclase leading to increased levels of intracellular cAMP. This in itself will activate cAMP dependent protein kinase A (PKA) and ultimately result in increased cytosolic levels of cAMP and Ca²⁺. The increased levels of cAMP and Ca²⁺ lead to the activation of several other additional kinases such as Ca²⁺/calmodulin-dependent kinase II (CAMKII) and p42/p44 mitogen activated kinases (MAPK). As a result the Ca²⁺/cAMP response element binding protein (CREB) is phosporylated and this in turn will regulate the transcription of genes containing cAMP response elements (CRE) in their promoter region. Examples of such genes shown to be involved in the modulation of CRH signaling include c-fos, macrophage migration-inhibitory factor gene Mif, orphan nuclear receptors Nurr77 and Nurr1.

Notwithstanding the fact that the downstream pathways for CRH activated receptors were extensively studied in AtT-20 cells, a cellular model of corticotrophs and led to the identification of a number of genes involved in the signaling cascade, a major area is unexplored. It was thus an object of the present invention to explore the transcriptional response to CRH stimulation at a genome wide level in order to identify further genes involved in the corticotropin-releasing hormone receptor activated gene network. The polypeptides thus identified and the polynucleotides encoding said polypeptides provide new chances for drug development as drug targets through screening techniques, or are useful in the diagnosis, prevention and/or treatment of depression.

### SUMMARY OF THE INVENTION

The present invention relates to the identification of a number of genes involved in the transcriptional response to CRH stimulation at a genome wide level. In particular to the identification of a number of hitherto unknown genes encoding a protein that modulates CRH signaling, having a sequence selected from the group consisting of SEQ ID No. 45, SEQ ID No. 47, SEQ ID No. 49 and functional analogs thereof.

In a further aspect the present invention relates to the recombinant use of the aforementioned nucleotide sequences, including vectors comprising these sequences, host cells containing a vector to encode for one of the aforementioned sequences as well as transgenic non-human animals comprising a polynucleotide or vector according to the invention.

It is also an object of the present invention to provide a number of genes that were hitherto not associated with CRH signaling and accordingly useful in methods to identify compounds, which modulate CHR signaling response in a cell or in diagnostic methods to identify CRH induced depression in an individual. In one embodiment the method to identify a compound capable to alter the CRH signaling response in a cell comprises, contacting said cell with CRH in the presence and absence of said compound and determine the expression level of a polynucleotide comprising a nucleic acid sequence selected from the group consisting of SEQ ID No. 1, SEQ ID No. 3, SEQ ID No. 5, SEQ ID No. 7, SEQ ID No. 9, SEQ ID NO. 11, SEQ ID N0 13, SEQ ID No.15, SEQ ID No 17, SEQ ID No 19, SEQ ID No. 21, SEQ ID No. 23, SEQ ID No. 25, SEQ ID No. 27, SEQ ID No. 29, SEQ ID NO. 31, SEQ ID No. 33, SEQ ID No. 35, SEQ ID No. 37, SEQ ID No. 39, SEQ ID No. 41, SEQ ID No. 43, SEQ ID No. 45, SEQ ID No. 47 or SEQ ID No. 49. In this screening method the expression levels are typically assessed using an oligonucleotide probe that binds to the aforementioned polynucleotides, preferably using array technology methods. Accordingly in a particular embodiment the present invention provides a method to identify compounds that modulate the CRH signaling response in a cell said method comprising, contacting said cell with CRH in the presence and absence of said compound; and determine the expression level of the polynucleotides having the nucleic acid sequences SEQ ID No. 1, SEQ ID No. 3, SEQ ID No. 5, SEQ ID No. 7, SEQ ID No.9, SEQ ID NO. 11, SEQ ID NO 13, SEQ ID No. 15, SEQ ID No 17, SEQ ID No 19, SEQ ID No. 21, SEQ ID No. 23, SEQ ID No. 25, SEQ ID No. 27, SEQ ID No. 29, SEQ ID NO. 31, SEQ ID No. 33, SEQ ID No. 35, SEQ ID No. 37, SEQ ID No. 39, SEQ ID No. 41, SEQ ID No. 43, SEQ ID No. 45, SEQ ID No. 47 and SEQ ID No. 49, wherein a change in expression profile of these sequences is indicative for a compound capable to alter the CRH signaling response in said cell.

### BRIEF DESCRIPTION OF THE DRAWING

Table 1: List of proteins which modulate CRH signaling.

FIG. 1: c-fos mRNA levels as assessed by quantitative RT-PCR normalized against β-actin mRNA levels (taken as 100%) in AtT-20 cells treated with DMSO, CRH, CRH+R121919 or R121919 for different time points (hours).

FIG. 2: Correspondence analysis applied on normalized microarray data for all time points and treatments. Squares depict different samples whereas circles depict genes. Distances between squares are a measure for similarity between samples. A positive association of a gene with a given sample (i.e. an upregulation of that gene in that particular sample) results in the positioning of the gene and sample on a common line through the centroid (depicted by a cross). Correspondence analysis clearly identifies time as the major discriminator between the samples. In addition the effect of treatment with CRH can be identified as most prominent in the early time points.

FIG. 3: A heat map depicting genes that are changed upon CRH treatment. Values were calculated by dividing the intensity of each sample by the intensity of the DMSO sample at the corresponding time point. These calculated ratio are converted into a color ramp using on a log₂ scale. In this way the different timing of induction of expression becomes apparent. Genes showing a 2-fold change after 30 minutes of treatment with CRH were called “early responders”, “intermediate responders” show a change after 1 to 2 hours of treatment and “late responders” show a response after 2 hours or more.

FIG. 4: Overview of a selection of genes induced by CRH grouped by pathway or function as discussed in the text. Values were calculated by dividing the intensity of each sample by the intensity of the DMSO sample at the corresponding time point. These calculated ratio are converted into a color ramp using on a log₂ scale and depicted in a heat map.

FIG. 5: Induction of Rgs2 by CRH in AtT-20 cells. Induction is calculated in comparison to levels observed in AtT-20 cells before any treatment. On top array data obtained for Rgs2 are shown. Below, levels of Rgs2 mRNA are shown as measured by quantitative RT-PCR on the same samples as used for array experiments and as measured on a repeated experiment.

### DETAILED DESCRIPTION

As used herein, the term “compound” or “agent” means a biological or chemical compound such as a simple or complex organic molecule, a peptide, a protein or an oligonucleotide. A “test compound” as used herein, refers to a “compound” or “agent” used in a method according to the invention to assess whether said compounds modulates CRH signalling activity.

“CRH signaling” as used herein refers to the cellular changes in gene transcription after activation of the corticotropin releasing hormone receptor by CRH in said cell. It induces a CRH specific gene expression profile. Changes at the transcriptional level can be assessed either at the protein level or at the gene, RNA level.

“CRH response activity” as used herein refers in general to the change of a detectable cellular parameter as a result of the exposure of said cell to CRH. Detectable cellular parameters include amongst others, changes in membrane potential, changes in enzyme activity of an enzyme that modulates CRH signalling a said cell, changes in expression levels of a protein according to the invention or changes in the amount of second messengers such as cGMP, cAMP, Ca²⁺ or IP₃.

The term “analog” or “functional analog” refers to a modified form of protein according to the invention in which at least one amino acid substitution has been made such that said analog retains substantially the same biological activity as the unmodified protein in vivo and/or in vitro.

The term “functional analog” is intended to include the “fragments,” “variants,” “degenerate variants,” “analogs” and “homologues” or to “chemical derivatives” of the polypeptides according to the invention. Useful chemical derivatives of polypeptide are well known in the art and include, for example covalent modification of reactive organic site contained within the polypeptide with a secondary chemical moiety. Well known cross-linking reagents are useful to react to amino, carboxyl, or aldehyde residues to introduce, for example an affinity tag such as biotin, a fluorescent dye, or to conjugate the polypeptide to a solid phase surface (for example to create an affinity resin)

Variant(s) of polynucleotides or polypeptides, as the term is used herein, are polynucleotides or polypeptides that differ from a reference polynucleotide or polypeptide, respectively. A variant of the polynucleotide may be a naturally occurring variant such as a naturally occurring allelic variant, or it may be a variant that is not known to occur naturally. (1) A polynucleotide that differs in nucleotide sequence from another, reference polynucleotide. Generally, differences are limited so that the nucleotide sequences of the reference and the variant are closely similar overall and, in many regions, identical. As noted below, changes in the nucleotide sequence of the variant may be silent. That is, they may not alter the amino acids encoded by the polynucleotide. Where alterations are limited to silent changes of this type a variant will encode a polypeptide with the same amino acid sequence as the reference. Also as noted below, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed above. (2) A polypeptide that differs in amino acid sequence from another, reference polypeptide. Generally, differences are limited so that the sequences of the reference and the variant are closely similar overall and, in many regions, identical. A variant and reference polypeptide may differ in amino acid sequence by one or more substitutions, additions, deletions, fusions and truncations, which may be present in any combination.

The terms “complementary” or “complementarity” as used herein refer to the capacity of purine and pyrimidine nucleotides to associate through hydrogen bonding to form double-stranded nucleic acid molecules. The following base pairs are related by complementarity: guanine and cytosine; adenine and thymine; and adenine and uracil. As used herein “complementary” means that the aforementioned relationship applies to substantially all base pairs comprising two single-stranded nucleic acid molecules over the entire length of said molecules. “Partially complementary” refers to the aforementioned relationship in which one of the two single-stranded nucleic acid molecules is shorter in length than the other such that a portion of one of the molecules remains single-stranded.

The term “conservative substitution” or “conservative amino acid substitution” refers to a replacement of one or more amino acid residue(s) in a parent protein without affecting the biological activity of the parent molecule based on the art recognized substitutability of certain amino acids (See e.g. M. Dayhoff, In Atlas of Protein Sequence and Structure, Vol. 5, Supp. 3, pgs 345-352, 1978).

“Fragment thereof” refers to a fragment, piece, or sub-region of a nucleic acid or protein molecule whose sequence is disclosed herein, such that said fragment comprises 5 or more amino acids, or 10 or more nucleotides that are contiguous in the parent protein or nucleic acid molecule.

“Functional fragment” as used herein, refers to an isolated sub-region, or fragment of a protein disclosed herein, or sequence of amino acids that, for example, comprises a functionally distinct region such as an active site for a receptor. Functional fragments may be produced by cloning technology, or as the natural products of alternative splicing mechanims.

The term “homolog” or “homologous” describes the relationship between different nucleic acid molecules or amino acid sequences in which said sequences or molecules are related by partial identity or similarity at one or more blocks or regions within said molecules or sequences. “Isolated nucleic acid compound” refers to any RNA or DNA sequence, however construed or synthesized, which is locationally distinct from its natural location.

A “nucleic acid probe” or “probe” as used herein is a labeled nucleic acid compound which hybridizes with another nucleic acid compound. “Nucleic acid probe” means a single stranded nucleic acid sequence that will hybridize with a single stranded target nucleic acid sequence. A nucleic acid probe may be an oligonucleotide or a nucleotide polymer. A “probe” will usually contain a detectable moiety which may be attached to the end(s) of the probe or be internal to the sequence of the probe.

The term “primer” is a nucleic acid fragment which functions as an initiating substrate for enzymatic or synthetic elongation of, for example, a nucleic acid molecule.

The term “hybridization” as used herein refers to a process in which a single-stranded nucleic acid molecule joints with a complementary strand through nucleotide base pairing.

The term “stringency” refers to hybridization conditions. High stringency conditions disfavor non-homologous base pairing. Low stringency conditions have the opposite effect. Stringency may be altered, for example, by temperature and salt concentration. “Stringent conditions” refers to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Further suitable hybridization conditions are described in the examples.

“Lower stringency conditions” include an overnight incubation at 37° C. in a solution comprising 6×SSPE (20×SSPE=3M NaCl; 0.2M NaH₂PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term “fusion protein” as used herein refers to protein constructs that are the result of combining multiple protein domains or linker regions for the purpose of gaining the combined functions of the domains or linker regions. This is may be accomplished by molecular cloning of the nucleotide sequences encoding such domains to produce a new polynucleotide sequence that encodes the desired fusion protein. Alternatively, creation of a fusion protein may be accomplished by chemically joining two proteins.

The term “linker region” or “linker domain” or similar such descriptive terms as used herein refers to polynucleotide or polypeptide sequence that are used in the construction of a cloning vector or fusion protein. Functions of a linker region can include introduction of cloning sites into the nucleotide sequence, introduction of a flexible component or space-creating region between two protein domains, or creation of an affinity tag for specific molecule interaction. A linker region may be introduced into a fusion protein resulting from choices made during polypeptide or nucleotide sequence construction.

Screening Methods

The present invention relates to screening methods to identify compounds that modulate corticotropin-releasing hormone (CRH) induced depression and stress. It is based on the identification of a number of genes as downstream modulators of the CRH activated CRH receptors. In particular this invention provides a method for identifying a compound capable to alter the CRH signalling response in a cell, said method comprising;
a) contacting said cell with CRH in the presence and absence of said compound;
b) determine the change at transcriptional level of at least one protein that modulates corticotropin releasing hormone (CRH) signaling in said cell; and
c) compare the transcriptional level of said protein in the presence and absence of said compound;
whereby the protein that modulates corticotropin releasing hormone (CRH) signaling is being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

To determine the change of transcription at the protein level one could determine the amount of said protein using art known techniques. For example using separation techniques such as isoelectric focusing or SDS-page in combination with protein staining techniques such as coomassie or silver staining. Alternatively, for proteins that are enzymes, the amount in a given solution or tissue extract can be measured or assayed in terms of the catalytic effect the enzyme produces, that is the conversion of its substrate into reaction product. For example, for kinases one may assess the kinase activity using a substrate comprising the kinase specific phosphorylation site and by measuring the phosphorylation of the substrate by incorporation of radioactive phosphate into the substrate. This assay may be perfomed both in the presence and absence of the compound to be tested. For proteins that are not enzymes, other quantification methods are required. For example transport proteins can be assayed by their binding to the molecule they transport and hormones and toxins by the biological effect they produce.

To assess changes in transcription at the gene level, RNA or cDNA may be used directly for detection or may be amplified enzymatically by using PCR or other amplification techniques prior to analysis. Preferably said analysis method comprises the use of a labelled oligonucleotide probe targeted to a suitable region of the gene.

Accordingly, in a preferred embodiment the level of gene transcription is assessed using a probe which binds to a polynucleotide encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

In another embodiment, an array of oligonucleotides probes comprising a nucleotide sequence encoding a protein that modulates CRH signalling or fragments thereof can be constructed to conduct efficient screening of gene expression. Array technology methods are well known and have general applicability and can be used to address a variety of questions in molecular genetics including gene expression, genetic linkage, and genetic variability (see for example: M. Chee et al., Science, Vol 274, pp 610-613 (1996)). It is thus an object of the present invention to provide a method for identifying a compound capable to alter the CRH signalling response in a cell, said method comprising; contacting said cell with CRH in the presence and absence of the compound to be tested; and determine the level of gene transcription using an array of oligonucleotide probes that bind to the polynucleotides encoding the group of polypeptides having the amino acid sequences SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

In an alternative embodiment, the method for identifying a compound capable to alter the CRH signalling response in a cell, comprises;
a) contacting said cell with CRH in the presence and absence of said compound; and
b) determine the expression level of a polynucleotide comprising a nucleic acid sequence selected from the group consisting of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 or SEQ ID NO:49.

To assess changes in expression levels, RNA or cDNA may be used directly for detection or may be amplified enzymatically by using PCR or other amplification techniques prior to analysis. Preferably said analysis method comprises the use of a labelled oligonucleotide probe targeted to a suitable region of the polynucleotiode. Accordingly, in a preferred embodiment the level of gene transcription is assessed using a probe which binds to a polynucleotide comprising a nucleic acid sequence selected from the group consisting of of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 or SEQ ID NO:49.

In another embodiment, an array of oligonucleotides probes comprising a nucleotide sequence encoding a protein that modulates CRH signalling or fragments thereof can be constructed to conduct efficient screening of gene expression. In this embodiment the invention provides a method for identifying a compound capable to alter the CRH signaling response in a cell, said method comprising, contacting said cell with CRH in the presence and absence of said compound; and determine the expression level of the poynucleotides having the nucleic acid sequences SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 and SEQ ID NO:49. In particular, using an array of oligonucleotide probes that bind to the polynucleotides having the nucleic acid sequences SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 and SEQ ID NO:49.

In another embodiment, an array of oligonucleotides probes comprising a nucleotide sequence encoding a protein that modulates CRH signalling or fragments thereof can be constructed to conduct efficient screening of gene expression. Array technology methods are well known and have general applicability and can be used to address a variety of questions in molecular genetics including gene expression, genetic linkage, and genetic variability (see for example: M. Chee et al., Science, Vol 274, pp 610-613 (1996)).

In case the genomic DNA is not used directly, mRNA may be isolated, and a first strand cDNA synthesis carried out. A second round of DNA synthesis can be carried out for the production of the second strand. Subsequently by the specific PCR amplification an isolated cDNA can be obtained. If desired the double-stranded cDNA can be cloned into any suitable vector, for example, a plasmid, thereby forming a cDNA libary. In analogy to the above, it is possible to screen cDNA libraries constructed in a bacteriophage or plasmid shuttle vector with a labeled oligonucleotide probe targeted to any suitable region of the gene encoding a protein that modulates CRH signalling. See e.g. PCR Protocols: A Guide to Method and Application, Ed. M. Innis et al., Academic Press (1990).

Methods for constructing cDNA libraries in a suitable vector such as a plasmid or phage for propagation in prokaryotic or eukaryotic cells are well known to those skilled in the art. [See e.g. Maniatis et al. Supra]. Suitable cloning vectors are well known and are widely available.

In a further embodiment changes in gene transcription are determined at mRNA level. Decreased or increased expression can be measured at the RNA level using any of the methods well known in the art for the quantitation of polynucleotides, such as, for example; nucleic acid amplification, for instance via PCR, RT-PCR; RNase protection; Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays. Assay techniques that can be used to determine the presence of protein derivatives or variants comprise amongst others mass spectrometry.

It is thus an object of the present invention to provide a method for identifying a compound capable to alter the CRH signalling response in a cell, said method comprising;
a) contacting said cell with CRH in the presence and absence of said compound;
b) determine the amount of at least one protein that modulates corticotropin releasing hormone (CRH) signaling in said cell; and
c) compare the amount of said protein in the presence and absence of said compound;
whereby the protein that modulates corticotropin releasing hormone (CRH) signaling is being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

Preferably, the method to assay the amount of protein that modulates CRH signaling is using an antibody which binds to a polypeptide comprising an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

Thus in another embodiment, this invention provides a monospecific antibody immunologically reactive with a protein that modulates CRH signalling said protein being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48. Antibodies generated against polypeptides of the present invention may be obtained by administering the polypeptides or epitope-bearing fragments, analogs or cells expressing these to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975)256:495-497), the trioma technique, the human B-cell hybridoma technique (Kozbor et al., Immunology Today (1983)4:72) and the EBV-hybridoma technique (Cole et al., MONOCLONAL ANTIBODIES AND CANCER THERAPY, pp. 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography.

Antibodies against polypeptides of the present invention may also be employed to treat the CRH metabolism related disorders such as CRH induced stress or depression amongst others.

To determine the amount of protein that modulates CRH signalling, the antibodies according to the invention are used in conventional immunological techniques. Suitable immunological techniques are well known to those skilled in the art and include for example, ELISA, Western Blot analysis, competitive or sandwich immunoassays and the like, as is otherwise well known they all depend on the formation of an antigen-antibody immune complex wherein for the purpose of the assay, the antibody can be detectable labeled with, e.g. radio, enzyme or fluorescent labels or it can be immobilized on insoluble carriers.

For example in an ELISA screening format the antibody is added to a solid phase (for example the bottom of a microplate) which is coated with either the protein or a peptide fragment thereof coupled to a carrier (such as BSA), and then, adding an anti-immunoglobin antibody (for example when the immunization is performed in mice, an anti-mouse immunoglobulin antibody is used, e.g. sheep-anti-mouse immunoglobulin (Ig)) conjugated with a detectable label such as an enzyme, preferably horseradish peroxidase, or a radioactive isotope such as ¹²⁵I.

It is thus an object of the invention to provide immunoassays for the determination or detection of proteins that modulate CRH signalling in a sample, the method comprising contacting the sample with an antibody to the proteins according to the invention and determining whether an immune complex is formed between the antibody and said protein. These methods can either be performed on tissue samples or body fluid samples and generally comprise obtaining a sample from the body of a subject; contacting said sample with an imaging effective amount of a detectably labeled antibody according to the invention; and detecting the label to establish the presence of proteins that modulate CRH signalling in the sample.

The measuring methods using the antibodies of the present invention are not particularly limited. Any measuring method may be used as long as the amount of antibodies, antigens or the antigens-antibody complexes corresponding to the amount of the antigens to be measured is detected by chemical or physical means, and calculated from standard curves prepared by the use of standard solutions containing the antigens in known amounts. For example, nephelometry, competitive methods, immunometric methods and sandwich methods are suitably used. With respect to sensitivity and specificity, it is particularly preferred to use sandwich methods described below.

In measuring methods using labelling substances, radioisotopes, enzymes, fluorescent substances, luminous substances, etc. are used as labelling agents. Examples of the radioisotopes include ¹²⁵I, ¹³¹I, ³H and ¹⁴C. Enzymes are usually made detectable by conjugation of an appropriate substrate that, in turn catalyzes a detectable reaction. Examples thereof include, for example, beta-galactosidase, beta-glucosidase, alkaline phosphatase, peroxidase and malate deydrogenase, preferably horseradish peroxidase. The luminous substances include, for example, luminol, luminol derivatives, luciferin, aequorin and luciferase. Further, the avidin-biotin systems can also be used for labelling the antibodies and immunogens of the present invention.

Accordingly, in a further aspect, the present invention provides for a method of identifying and obtaining compounds that alter the CRH signalling response activity in a cell, comprising:
a) contacting a cell which expresses at least one protein comprising an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48, with said test compound; and
b) compare the CRH response activity of said cell in the presence and absence of said compound.

Changes in membrane potential can be measured using conventional electrophysiological techniques and when they become available, using novel high throughput methods currently under development. Since the change in membrane potential are normally the result of ion fluxes, as an alternative approach, changes in membrane potential can be measured indirectly through the change in intracellular ion concentrations using ion-sensitive fluorescent dyes, including fluo-3, fluo-4, fluo-5N, fura red, Sodium Green, SBFI and other similar probes from suppliers including Molecular Probes. Other fluorescent dyes, from suppliers including Molecular Probes, such as DIBAC₄₍₃₎ or Di-4-Anepps can detect membrane potential changes. For example calcium and sodium ion fluxes can thus be characterised in real time, using fluorometric and fluorescence imaging techniques, including fluorescence microscopy with or without laser confocal methods combined with image analysis algorithms.

In a preferred embodiment this assay is based around an instrument called a FLuorescence Imaging Plate Reader ((FLIPR), Molecular Devices Corporation). In its most common configuration, it excites and measures fluorescence emitted by fluorescein-based dyes. It uses an argon-ion laser to produce high power excitation at 488 nm of a fluorophore, a system of optics to rapidly scan the over the bottom of a 96-/384-well plate and a sensitive, cooled CCD camera to capture the emitted fluorescence. It also contains a 96-/384-well pipetting head allowing the instrument to deliver solutions of test agents into the wells of a 96-/384-well plate. The FLIPR assay is designed to measure fluorescence signals from populations of cells before, during and after addition of compounds, in real time, from all 96-/384-wells simultaneously.

It is thus an object of the present invention to provide a FLIPR assay used to screen for and characterise compounds functionally active in modulating CRH response in cells, said cells expressing a protein selected from the group consisting of SEQ ID 26, SEQ ID NO.28, SEQ ID NO.30, SEQ ID NO.32 and SEQ ID NO.34.

In an alternative embodiment, the activity of the cell may be assessed using electrophysiological methods. Therefore, proteins modulating CRH signalling in a cell can be characterised using whole cell and single channel electrophysiology.

It is thus a further object of this invention to provide a screening method to identify compounds which modulate CRH signalling response activity in a cell, said method comprising;

(a) contacting a host cell expressing a protein selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48, with a compound to be tested;

(b) measuring the effect of the test compound on the membrane potential of said cell using electrophysiological techniques; and

(c) compare the CRH response activity of said cell in the presence and absence of said compound. Alternatively, the host cells in the aforementioned screening method express a protein selected from the group consisting of SEQ ID No. 26, SEQ ID No.28, SEQ ID No. 30, SEQ ID No. 32 and SEQ ID NO.34.

In a preferred embodiment the host cells are Xenopus oocytes and the electrophysiological measurement consists of measuring the membrane current using the voltage clamp technique at distinct membrane potentials.

Changes in enzyme activity of an enzyme that modulates CRH signalling a said cell, can generally be measured or assayed in terms of the catalytic effect the enzyme produces, that is the conversion of its substrate into reaction product. For example, for kinases one may assess the kinase activity using a substrate comprising the kinase specific phosphorylation site and by measuring the phosphorylation of the substrate. Similarly for phosphatases one may assess the phosphatase activity using a phosphorylated substrate and by measuring the dephosphorylation of the substrate. These assays may be performed both in the presence and absence of the compound to be tested.

It is thus an object of the present invention to provide a method for identifying a compound capable to alter the CRH signalling response activity in a cell, said method comprising;
a) contacting a mixture comprising a kinase selected from the group consisting of SEQ ID NO.10, SEQ ID NO.12, SEQ ID NO.14, SEQ ID No16 and SEQ ID NO.18, with a source of phosphate and a suitable kinase substrate;
b) incubating said mixture in the presence or absence of a said compound and;

measuring the level of phosphorylation of said substrate in the presence of said compound compared to the level of phosphorylation of said substrate in the absence of said test compound.

In the assay of the invention, the kinase may be provided as a protein or it may be provided in the assay mixture as an mRNA encoding said kinase. When the assay comprises cell-free components, the kinase is provided as the protein. When the assay is conducted in the milieu of a cell, the kinase may be provided as either the protein or as an mRNA encoding said kinase, wherein, in order that the kinase be available in the assay, the mRNA is translated and kinase protein is thereby produced. It will be apparent from the Examples provided herein that it is a simple matter to obtain mRNA specifying the kinase and inject the mRNA into a cell for production of the kinase protein. The kinase may also be provided by expression of a plasmid, which encodes the kinase protein. Standard molecular biology techniques may be used to construct operable plasmids encoding the kinase protein and to express the plasmid in cells (Sambrook, et al., 1989, In: Molecular Cloning: A Laboratory Manual, Cold Spring Harbor Laboratory, New York).

As discussed herein, the method of identifying a kinase modulator may be performed either in vitro wherein the assay mixture is cell-free, in vitro wherein live cells are included in the assay, or in vivo in an animal. Thus, in one aspect of the invention, the mixture is contained within a eukaryotic cell and the method of the invention may be performed wherein some of the components of the assay mixture may be provided exogenously to a cell my microinjection of the components therein, and some of the components may be endogenous in the cell.

The term “endogenous in the cell” as used herein, means that the component is naturally produced in the subject cell.

The term “exogenous to the cell” as used herein, means that the component is not found naturally in the subject cell, or is found therein at a low level, and is added thereto. When the method of the invention is performed using a eukaryotic cell, one or more of the kinase protein, the kinase substrate and the test compound may be injected into the eukaryotic cell prior to the incubation. The cell so injected is then incubated under conditions that facilitate protein kinase activity and the level of protein kinase activity is subsequently measured following the incubation period using the assays described herein.

The eukaryotic cell that is useful in the methods of the invention may be any one of a Xenopus laevis oocyte, a Xenopus laevis embryo cell, a mammalian cell (such as a I OTI/2 cell), a Drosophila melanogaster S2 cell, a Dictyostefium discoideum cell and a yeast cell. Still more preferably, the eukaryotic cell is the murine pituitary corticotroph-derived adenoma cell line cell AtT-20.

The source of phosphate for use in the methods of the invention may be any common source of phosphate, including, but not limited to, a nucleotide triphosphates such as, but not limited to, ATP or GTP. In a preferred embodiment, the phosphate source has bound thereon a detectable label which label is transferred with the phosphate group to the kinase substrate during the reaction. In this manner, phosphorylated kinase substrate may be distinguished from non-phosphorylated kinase substrate in that the phosphorylated substrate will contain the detectable label whereas the non-phosphorylated substrate will not contain the label. In another embodiment, the phosphate source does not have bound thereon a detectable label; instead, phosphorylated kinase substrate may be distinguished from non-phosphorylated kinase substrate, for instance by recognition of one form of the substrate, but not the other, by an antibody.

The detectable label, which is useful in the methods of the invention may include any known or heretofore unknown detectable label which is transferred to the kinase substrate upon transfer of a phosphate group thereto as a result of protein kinase activity.

Labels which are useful include, but are not limited to, radioactive labels, such as γ³²P, ³¹S, and non-radioactive labels, such as biotin and the like.

In another embodiment, the present invention to provide a method for identifying a compound capable to alter the CRH signalling response activity in a cell, said method comprising;
a) contacting a mixture comprising a phosphatase selected from the group consisting of SEQ ID NO.36 and SEQ ID NO.38, and a suitable phosphorylated substrate;
b) incubating said mixture in the presence or absence of a said compound and;

measuring the level of phosphorylation of said substrate in the presence of said compound compared to the level of phosphorylation of said substrate in the absence of said test compound.

As for the kinase assay, the phosphatase in the assay of the invention, may be provided as a protein or it may be provided in the assay mixture as an mRNA encoding said phosphatase. The phosphorylated substrate is typically labeled with a detectable phosphate residue. Labels which are useful include, but are not limited to, radioactive labels, such as γ³²P, ³¹S, and non-radioactive labels, such as biotin and the like. For use in a phosphatase activity assay, the substrate preferably consists of a peptide substrate, phosphorylated at a tyrosine or serine residue, typically labeled with γ³²P. In general, phosphorylation may be accomplished in a variety of ways. Typically, a protein tyrosine kinase is used. For example, a soluble EGF-receptor kinase in combination with sup.32 P-labeled ATP may be used to phosphorylate a tyrosine residue on a peptide of the present invention. Such a phosphorylation reaction is typically allowed to proceed for about 2 hours at 30.degree. C. or overnight at room temperature. Phosphorylated peptide, hereinafter referred to as “phosphopeptide”, is then purified from a phosphorylation reaction mixture. For example, peptide may be separated from a reaction mixture by addition of trichloroacetic acid and centrifugation, whereby the peptide remains in the supernate. The peptide is generally further purified by column chromatography, e.g., on C18. Purified phosphorylated peptide may be lyophilized and stored at −20.degree. C. prior to use.

Following incubation, phosphopeptide which is not dephosphorylated (“non-dephosphorylated phosphopeptide”) is separated from radioactivity released by dephosphorylation of phosphopeptide (i.e., from free radioactive phosphorus released by dephosphorylation). As used herein, the term “radioactive phosphorous” includes all forms in which a radioactive phosphorus atom may be present on a tyrosine residue and removed by dephosphorylation, e.g., as a phosphate group. Typically, separation of non-dephosphorylated phosphopeptide from free radioactive phosphorus released by dephosphorylation of phosphopeptide is effected by centrifugation, following termination of the dephosphorylation reaction by the addition of substances including nonradioactive phosphates and charcoal. Radioactivity in the supernate is determined by means well known to those of ordinary skill in the art. Based upon the amount of radioactivity added to the assay mixture initially via the phosphopeptide and the amount of radioactivity detected at the end of the assay as radioactivity released by dephosphorylation, the phosphatase enzymatic activity of the sample assayed may be calculated.

It is also an embodiment of the present invention to provide a method for identifying a compound capable to alter the CRH signalling response activity in a cell, said method comprising;
a) contacting a cell which expresses at least one protein comprising an amino acid sequence selected from the group consisting of SEQ ID NO.26, SEQ ID NO.28, SEQ ID NO.30, SEQ ID NO.32 and SEQ ID NO.34 with said test compound; and
b) compare the levels of a second messenger, such as cAMP, cGMP, Ca²⁺ or IP₃ in said cell, in the presence and absence of said compound.

Levels of second messengers can be determined using art known techniques either in whole cells or cellular extracts comprising one of the aforementioned proteins.

A further method to identify a compound capable to alter CRH signalling in a cell is based on the use of a gene, such as a reporter gene, operably linked to a gene promoter or regulatory sequence element thereof characterized in that said gene promoter or regulatory sequence element comprises a transcription factor binding site, wherein said transcription factor is capable of modulating CRH signalling in a cell. In a preferred embodiment the transcription factor capable of modulating CRH signalling in a cell is being selected from SEQ ID NO.2, SEQ ID NO.4, SEQ ID NO.6 and SEQ ID NO.8. Accordingly, the present invention provides a recombinant DNA molecule comprising the gene promoter region as defined above. In the said recombinant DNA molecule, the promoter region can be operably linked to a nucleic acid molecule encoding a detectable product, such as a reporter gene. The term “operably linked”, as used herein, means functionally fusing a promoter with a gene in the proper frame to express the gene under control of the promoter. As used herein, the term “reporter gene” means a gene encoding a gene product that can be identified using simple, inexpensive methods or reagents and that can be operably linked to the promoter region or an active fragment thereof. Reporter genes such as, for example, a firefly luciferase, □-galactosidase, alkaline phosphatase, the bacterial chloramphenicol acetyl transferase or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra). In a preferred embodiment, the reporter gene is the firefly luciferase gene. The invention also provides a vector comprising the recombinant DNA molecule as defined above, as well as a host cell stably transformed with such a vector, or generally with the recombinant DNA molecule according to the invention. The term “vector” refers to any carrier of exogenous DNA that is useful for transferring the DNA into a host cell for replication and/or appropriate expression of the exogenous DNA by the host cell. Accordingly, in a specific embodiment said vector is an expression vector such as pGL31uc, pBLCAT5 (LMBP 2451), pGMCSFlacZ (LMBP 2979), pEGFP or pSEAPbasic (DMB 3115), wherein LMBP and DMB numbers refer to the accession numbers of these expression vectors at the Belgian Co-ordinated Collections of Micro-organisms.

In another aspect, the invention provides a method for identification of a compound modulating CRH signalling activity, said method comprising the steps: (i) contacting a candidate agent with a gene promoter region as defined above; and (ii) determining whether said candidate agent modulates expression of the detectable product, such modulation being indicative for an agent capable of modulating CRH signalling activity. The detectable product refers either to the gene encoded protein or to the product of a reporter gene such as luciferase, β-galactosidase or green fluorescent protein. Methods to quantify the detectable products are generally known in the art and include amongst others the use of a calorimetric substrate if the expression product is an enzyme, the use of specific antibodies in an RIA or ELISA assay or the measurement of the level of mRNA transcribed from genes operably linked to the promoter, wherein said mRNA can be measured either directly or indirectly using standard procedures. Preferably, the gene promoter comprising a transcription factor binding site for a transcription factor selected from the group consisting of SEQ ID NO.2, SEQ ID NO.4, SEQ ID NO.6 and SEQ ID NO.8.

Identification of the Nucleic Acid Sequences Encoding Proteins Capable of Modulating CRH Signaling.

In another aspect the present invention relates to isolated and purified nucleic acid molecules which encodes proteins capable of modulating CRH signalling, wherein said nucleic acid molecule is either RNA, DNA, cDNA or genomic DNA.

In particular, the present invention encompasses an isolated and purified nucleic acid molecule comprising a member selected from a group consisting of:
(a) a nucleic acid molecule encoding a protein that modulates CRH signalling having at least a 70% identity to a polypeptide comprising an amino acid sequence selected from the group consisting of SEQ ID No.46 and SEQ ID No.48;
(b) a nucleic acid molecule which is complementary to the polynucleotide of (a);
(c) a nucleic acid molecule comprising at least 15 sequential bases of the polynucleotide of (a) or (b);
(d) a nucleic acid molecule that hybridizes under stringent conditions to the polynucleotide molecule of (a) or (b); and
(e) a nucleic acid molecule encoding a protein that modulates CRH signalling comprising a nucleotide sequence of which is degenerated as a result of the genetic code to a nucleotide sequence of a polynucleotide of any of (a) to (d).

Those skilled in the art will recognize that owing to the degeneracy of the genetic code, numerous “silent” substitutions of nucleotide base pairs could be introduced into the sequences identified as SEQ ID NO:45, SEQ ID NO 47 or SEQ ID NO:49 without altering the identity of the encoded amino acid(s) or protein products. All such substitutions are intended to be within the scope of the invention.

In a further aspect, the present invention relates to Human purine permease polynucleotides. Such polynucleotides include isolated polynucleotides comprising a nucleotide sequence encoding a polypeptide which has at least 70% identity, preferably at least 80% identity, more preferably at least 90% identity, yet more preferably at least 95% identity, to the amino acid sequence selected from the group consisting of SEQ ID NO:46 and SEQ ID NO:48, over the entire length said amino acid sequence. In this regard, polypeptides which have at least 97% identity are highly preferred, while those with at least 98-99% are more highly preferred, and those with at least 99% identity are most highly preferred. Such polynucleotides include a polynucleotide consisting essentially of a polynucleotide sequence selected from SEQ ID No.45, SEQ ID No.47 or SEQ ID No.49.

Accordingly, in a further aspect, the present invention provides for an isolated polynucleotide, comprising:

a) a nucleotide sequence encoding a polypeptide which has at least 70% identity, preferably at least 80% identity, more preferably at least 90% identity, more preferably at least 90% identity, yet more preferably at least 90% identity, yet more preferably at least 95% identity, even more preferably at least 97-99% identity to an amino acid sequence selected from the group consisting of SEQ ID No. 46 and SEQ ID No.48;

b) a nucleotide sequence which has at least 70% identity, preferably at least 80% identity, more preferably at least 90% identity, more preferably at least 90% identity, Yet more preferably at least 90% identity, yet more preferably at least 95% identity, even more preferably at least 97-99% identity, to a polynucleotide selected from the group consisting of SEQ ID No.45, SEQ ID No.47 and SEQ ID No.49, over the entire length of said polynucleotide;

c) a nucleotide sequence which has at least 70% identity, preferably at least 80% identity, more preferably at least 90% identity, more preferably at least 90% identity, Yet more preferably at least 90% identity, yet more preferably at least 95% identity, even more preferably at least 97-99% identity a polynucleotide selected from the group consisting of SEQ ID No.45, SEQ ID No.47 and SEQ ID No.49, over the entire coding region of said polynucleotide; and

d) a nucleotide sequence consisting of a polynucleotide selected from the group consisting of SEQ ID No.45, SEQ ID No.47 and SEQ ID No.49.

The polynucleotides as outlined above are in particular provided for use in a screening method or diagnostic method according to the invention. In particular to identify capable of modulating CRH signaling in a cell or to diagnose altered CRH metabolism in an individual.

Identity or similarity, as known in the art, are relationships between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity and similarity between two polynucleotide or two polypeptide sequences, both terms are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., (1988) SIAM J. Applied Math., 48, 1073. Methods commonly employed to determine identity or similarity between sequences include, but are not limited to those disclosed in Carillo, H., and Lipman, D., (1988) SIAM J. Applied Math., 48, 1073. Preferred methods to determine identity are designed to give the largest match between the sequences tested. Methods to determine identity and similarity are codified in computer programs. Preferred computer program methods to determine identity and similarity between two sequences include, but are not limited to, GCG program package (Devereux, J., et al., (1984) Nucleic Acids Research 12(1), 387), BLASTP, BLASTN, and FASTA (Atschul, S. F. et al., (1990) J. Molec. Biol. 215, 403).

The nucleic acid sequence encoding a protein capable of modulating CRH activity, or fragment thereof, can be isolated from a tissue in which said gene is expressed, such as but not limited to, brain, hart, kidney, pancreas, liver and skin. Said sequence can also be isolated from mammals other than human and mouse. Other cells and cell lines may also be suitable for use to isolate mammalian purine permease cDNA. Selection of suitable cells may be done by screening for CRH modulating activity in cell extracts or in whole cell assays, as described herein. Cells that possess CRH modulating activity in any one of these assays may be suitable for the isolation of purine permease DNA or mRNA.

Any of a variety of procedures known in the art may be used to moleculary clone DNA encoding a protein according to the invention. In one method, mRNA is isolated, and first strand cDNA synthesis is carried out. A second round of DNA synthesis can be carried out for the production of the second strand. Subsequently by the specific PCR amplification of DNA fragments through the design of degenerate oligonucleotide primers from the amino acid sequence of the purified protein that modulates CRH signalling, an isolated cDNA can be obtained. If desired the double-stranded cDNA can be cloned into any suitable vector, for example, a plasmid, thereby forming a cDNA libary. Another method is to screen cDNA libraries constructed in a bacteriophage or plasmid shuttle vector with a labeled oligonucleotide probe targeted to any suitable region of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO.37, SEQ ID NO.39, SEQ ID NO.41, SEQ ID NO.43, SEQ ID NO.45, SEQ ID NO.47 or SEQ ID NO:49. See e.g. PCR Protocols: A Guide to Method and Application, Ed. M. Innis et al., Academic Press (1990).

Methods for constructing cDNA libraries in a suitable vector such as a plasmid or phage for propagation in prokaryotic or eukaryotic cells are well known to those skilled in the art. [See e.g. Maniatis et al. Supra]. Suitable cloning vectors are well known and are widely available.

It is readily apparent to those skilled in the art that other types of libraries, as well as libraries constructed from other cells or cell types, may be useful for isolating the nucleic acid sequences according to the invention. Other types of libraries include, but are not limited to, cDNA libraries derived from other cells, from organisms other than human and mouse, and genomic DNA libraries that include YAC (yeast artificial chromosome) and cosmid libraries. Construction of genomic DNA libraries can be performed by standard techniques well known in the art. Well known genomic DNA library construction techniques can be found in T. Maniatis et al. Molecular Cloning: A Laboratory Manual, 2d Ed. Chap. 14 (1989).

The skilled artisan will appreciate that, in many cases, an isolated cDNA sequence will be incomplete, in that the region coding for the polypeptide is short at the 5′ end of the cDNA. This is a consequence of reverse transcriptase, an enzyme with inherently low ‘processivity’ (a measure of the ability of the enzyme to remain attached to the template during the polymerisation reaction), failing to complete a DNA copy of the mRNA template during the 1^{st} strand cDNA synthesis.

There are several methods available and well known to those skilled in the art to obtain full-length cDNAs, or extend short cDNAs, for example those based on the method of Rapid Amplification of cDNA ends (RACE) (Frohman et al., 1988, PNAS USA 85, 8998-9002), or recent modifications of this technique, exemplified by the Marathon™ technology (Clontech Laboratories Inc.).

In order to clone the polynucleotide encoding a protein according to the invention by the above methods, the amino acid sequence of the polypeptide encoded by said nucleic acid sequence may be necessary. To accomplish this, the proteins according to the invention, may be purified and partial amino acid sequence determined by automated sequenators. It is not necessary to determine the entire amino acid sequence, but the linear sequence of two regions of 6 to 8 amino acids from the protein is determined for the production of primers for PCR amplification of a partial DNA fragment.

Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the polynucleotide sequences according to the invention and will be capable of hybridizing to DNA encoding the desired protein, even in the presence of DNA oligonucleotides with mismatches. DNA isolated by these methods can be used to screen DNA libraries from a variety of cell types, from invertebrate and vertebrate sources, and to isolate homologous genes.

Polypeptides

In a further embodiment this invention relates to a polypeptide in a substantially pure form which modulate CRH signalling wherein said polypeptide is encoded by an isolated and purified nucleic acid molecule according to the invention. In a preferred embodiment the polypeptide has the amino acid sequence selected from the group consisting of SEQ ID NO 46, SEQ ID NO 48 and functional analogs thereof.

The protein according to the invention includes all possible amino acid variants encoded by the nucleic acid according to the invention including a polypeptide encoded by said molecule and having conservative amino acid changes.

Those skilled in the art will recognize that the protein which modulate CRH signalling could be obtained by a plurality of recombinant DNA techniques including, for example, hybridization, polymerase chain reaction (PCR) amplification, or de novo DNA synthesis (See e.g., T. Maniatis et al. Molecular Cloning: A Laboratory Manual, 2d Ed. Chap. 14 (1989)).

Purified biologically active protein that modulates CRH signallings may have several different physical forms. The polypeptides according to the invention may exist as full-length nascent or unprocessed polypeptides, or as partially processed polypeptides or combinations of processed polypeptides. The full-length nascent polypeptide may be post-translationally modified, amongst other, by specific proteolytic cleavage events that result in the formation of fragments of the full-length nascent polypeptide. A fragment, or physical association of fragments may have the full biological activity associated with proteins according to the invention; however, the degree of CRH modulating activity may vary between individual fragments.

Also preferred in this aspect of the invention are fragments characterized by structural or functional attributes of the polypeptide. Preferred embodiments of the invention in this regard include fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, high antigenic index regions of the polypeptide of the invention, and combinations of such fragments. Preferred regions are those that mediate activities of the polypeptides of the invention. Most highly preferred in this regard are fragments that have a chemical, biological or other activity of the response regulator polypeptide of the invention, including those with a similar activity or an improved activity, or with a decreased undesirable activity.

Recombinant Expression of Polynucleotides Encoding Protein which Modulate CRH Activity

In another embodiment polynucleotides according to the invention may be recombinantly expressed by molecular cloning into an expression vector containing a suitable promoter and other appropriate transcription regulatory elements, and transferred into prokaryotic or eukaryotic host cells to produce a protein that modulates CRH signalling. Techniques for such manipulations are fully described in Maniatis, T, et al., supra, and are well known in the art.

Therefore, in a further aspect this invention provides an expression vector for expression of a protein that modulates CRH signalling in a recombinant host, wherein said vector contains a nucleic acid sequence encoding a protein that modulates CRH signalling and functional analogs thereof. In a more preferred aspect of this invention this expression vector contains a nucleic acid molecule encoding a protein that modulates CRH signalling, having a nucleotide sequence selected from a group consisting of: SEQ ID NO:45, SEQ ID NO 47, SEQ ID NO:49 and functional analogs thereof or contains genomic DNA encoding a protein that modulates CRH signalling.

Expression vectors are defined herein as DNA sequences that are required for the transcription of cloned copies of genes and the translation of their mRNAs in an appropriate host. Such vectors can be used to express eukaryotic genes in a variety of hosts such as bacteria including E. coli, cyanobacteria, plant cells, insect cells, amphibian cells, fungal cells including yeast cells, and animal cells.

Specifically designed vectors allow the shuttling of DNA between hosts such as bacteria-yeast or bacteria-animal cells or bacteria-fungal cells or bacteria-invertebrate cells. An appropriately constructed expression vector may contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency. Expression vectors may include, but are not limited to, cloning vectors, modified cloning vectors, specifically designed plasmids or viruses.

The isolated and purified nucleic acid molecules, according to the invention, encoding a protein which modulates CRH signalling may be cloned into an expression vector for expression in a recombinant host cell. Recombinant host cells may be prokaryotic or eukaryotic, including but not limited to bacteria such as E. coli, fungal cells such as yeast, amphibian cells such as Xenopus oocytes, mammalian cells including but not limited to cell lines of human, bovine, porcine, monkey and rodent origin, and insect cells including but not limited to Drosophila- and silkworm-derived cell lines. Cell lines derived from mammalian species which may be suitable and which are commercially available, include but are not limited to, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, neuroblastoma, glial cells and HEK-293 (ATCC CRL1573).

Therefore, in a further embodiment this invention relates to a recombinant host cell containing a recombinantly cloned nucleic acid molecule encoding a protein that modulates CRH signalling or functional analog thereof. In a further aspect the recombinant host cell according to the invention contains a nucleic acid molecule which is either genomic DNA or has a nucleotide sequence selected from a group consisting of: (SEQ ID NO:45); (SEQ ID NO 47); (SEQ ID NO:49); and functional analogs thereof.

The expression vector may be introduced into host cells via any one of a number of techniques including but not limited to transformation, transfection, protoplast fusion, lipofection, and electroporation. The expression vector-containing cells are clonally propagated and analyzed to determine whether they produce a protein that modulates CRH signalling. Identification of permeases expressing host cell clones may be done by several means, including but not limited to immunological reactivity with antibodies directed against the polypeptides according to the invention, and the presence of host cell-associated mammalian purine permease activity.

Thus, the present invention also relates to a process for expression of protein that modulates CRH signalling in a recombinant host cell, comprising culturing the host cells according to the invention under conditions which allow expression of the protein that modulates CRH signalling from the expression vector as outlined herein. The proteins of this invention may be synthesized either by direct expression or as a fusion protein comprising the protein of interest as a translational fusion with another protein or peptide that may be removable by self, enzymatic or chemical cleavage. Therefore, in a particular embodiment this invention provides the proteins according to the invention wherein said polypetides are part of a fusion protein.

It is often observed in the production of certain peptides in recombinant systems that expression as a fusion protein prolongs the life span, increases the yield of the desired peptide, or provides a convenient means of purifying the protein. This is particularly relevant when expressing mammalian proteins in prokaryotic hosts. A variety of peptidases (e.g. enterokinase and thrombin), which cleave a polypeptide at specific sites or digest, the peptides from the amino or carboxy termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., P. Carter, “Site Specific Proteolysis of Fusion Proteins”, Chapter 13, in Protein Purification: From Molecular Mechanisms to Large Scale Processes, American Chemical Society, Washington, D.C. (1990).

Furthermore, one could use, e.g., a mammalian cell that already comprises in its genome a nucleic acid molecule encoding a protein that modulates CRH signalling as described above, but does not express the same or not in an appropriate manner due to, e.g., a weak promoter, and introduce into the mammalian cell a regulatory sequence such as a strong promoter in close proximity to the endogenous nucleic acid molecule encoding said purine permease polypeptide so as to induce expression of the same.

As such a recombinant host cell containing a polynucleotide encoding a protein which modulates CRH signalling under the control of a heterologous transcription and/or regulatory sequence or protein, would be another embodiment of this invention.

In this context the term “regulatory sequence” denotes a nucleic acid molecule that can be used to increase the expression of the purine permease polypeptide, due to its integration into the genome of a cell in close proximity to the CRH modulating protein-encoding gene. Such regulatory sequences comprise promoters, enhancers, inactivated silencer intron sequences, 3′UTR and/or 5′UTR coding regions, protein and/or RNA stabilizing elements, nucleic acid molecules encoding a regulatory protein, e.g., a transcription factor, capable of inducing or triggering the expression of the CRH modulating protein-encoding gene or other gene expression control elements which are known to activate gene expression and/or increase the amount of the gene product. The introduction of said regulatory sequence leads to increase and/or induction of expression of polypeptides, which modulate CRH signalling, resulting in the end in an increased amount of said polypeptides in the cell. Thus, the present invention is aiming at providing de novo and/or increased expression of polypeptides that modulate CRH signalling.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis, Basic Methods In Molecular Biology (1986). It is specifically contemplated that polypeptides, which modulate CRH signalling may in fact be expressed by a host cell lacking a recombinant vector.

In addition, expression of polynucleotides according to the invention may also be performed using in vitro produced synthetic mRNA. Synthetic mRNA or mRNA isolated from cells capable of modulating CRH signaling can be efficiently translated in various cell-free systems, including but not limited to wheat germ extracts and reticulocyte extracts, as well as efficiently translated in cell based systems, including but not limited to microinjection into frog oocytes, with microinjection into frog oocytes being generally preferred.

Transgenic Non-Human Animals

The present invention also relates to a method for the production of a transgenic non-human animal, preferably transgenic mouse, comprising introduction of a polynucleotide or vector of the invention into a germ cell, an embryonic cell, stem cell or an egg or a cell derived therefrom. The non-human animal can be used in accordance with a screening method of the invention described herein and may be a non-transgenic healthy animal, or may have a phosphate uptake or reabsorption disorder, preferably a disorder caused by at least one mutation in the protein that modulates CRH signalling. Such transgenic animals are well suited for, e.g., pharmacological studies of drugs in connection with mutant forms of the above described polypeptides. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra.

Preferably, the transgenic non-human animal of the invention further comprises at least one inactivated wild type allele of the corresponding mammalian CRH modulating protein-encoding gene; see supra. This embodiment allows for example, the study of the interaction of various mutant forms of polypeptides according to the invention on the onset of the clinical symtoms of disease related to disorders in CRH metabolism. All the applications that have been herein before discussed with regard to a transgenic animal also apply to animals carrying two, three or more transgenes; e.g. encoding neutral endopeptidase (NEP). It might be also desirable to inactivate protein that modulates CRH signalling expression or function at a certain stage of development and/or life of the transgenic animal. This can be achieved by using, for example, tissue specific, developmental and/or cell regulated and/or inducible promoters which drive the expression of, e.g., an antisense or ribozyme directed against the RNA transcript encoding the protein capable of modulating CRH signalling; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of the mutant protein that modulates CRH signalling may be controlled by such regulatory elements.

Furthermore, the invention also relates to a transgenic mammalian cell which contains (preferably stably integrated into its genome) a nucleic acid molecule according to the invention or part thereof, wherein the transcription and/or expression of the nucleic acid molecule or part thereof leads to reduction of the synthesis of a protein that modulates CRH signalling.

In a preferred embodiment, the reduction is achieved by an anti-sense, sense, ribozyme, co-suppression and/or dominant mutant effect. “Antisense” and “antisense nucleotides” means DNA or RNA constructs which block the expression of the naturally occurring gene product.

The provision of the polynucleotide according to the invention opens up the possibility to produce transgenic non-human animals with a reduced level of the protein as described above and, thus, with a defect in phosphate metabolism. Techniques how to achieve this are well known to the person skilled in the art. These include, for example, the expression of antisense-RNA, ribozymes, of molecules which combine antisense and ribozyme functions and/or of molecules which provide for a co-suppression effect; see also supra. When using the antisense approach for reduction of the amount of protein that modulates CRH signallings in cells, the nucleic acid molecule encoding the antisense-RNA is preferably of homologous origin with respect to the animal species used for transformation. However, it is also possible to use nucleic acid molecules which display a high degree of homology to endogenously occurring nucleic acid molecules encoding a protein that modulates CRH signalling. In this case the homology is preferably higher than 80%, particularly higher than 90% and still more preferably higher than 95%. The reduction of the synthesis of a protein according to the invention in the transgenic mammalian cells can result in an alteration in, e.g., adenine reabsorption. In transgenic animals comprising such cells this can lead to various physiological, developmental and/or morphological changes.

Thus, the present invention also relates to transgenic non-human animals comprising the above-described transgenic cells. These may show, for example, a deficiency in CRH metabolism compared to wild type animals due to the stable or transient presence of a foreign DNA resulting in at least one of the following features:
(a) disruption of (an) endogenous gene(s) encoding a protein capable of modulating CRH signalling;
(b) expression of at least on antisense RNA and/or ribozyme against a transcript comprising a polynucleotide of the invention;
(c) expression of a sense and/or non-translatable mRNA of the polynucleotide of the invention;
(d) expression of an antibody of the invention;
(e) incorporation of a functional or non-functional copy of the regulatory sequence of the invention; or
(f) incorporation of a recombinant DNA molecule or vector of the invention.

With the polypeptides, their encoding polynucleotides and vectors of the invention, it is now possible to study in vivo and in vitro the efficiency of drugs in relation to particular mutations in protein that modulates CRH signallings of a patient and the affected phenotype. Furthermore, mutant forms of polypeptides of the invention can be used to determine the pharmacological profile of drugs and for the identification and preparation of further drugs which may be effective for the treatment of disorders related to the CRH metabolism, in particular for the amelioration of CRH induced stress or depression.

It will thus be appreciated that the present invention also relates to a method for preventing, treating or ameliorating a medical condition related to a disorder of CRH metabolism including CRH receptor related disorders which comprises administering to a mammalian subject a therapeutically effective amount of the polypeptides, the polynucleotides or the vectors encoding a protein capable of modulating CRH signalling of the present invention.

Diagnostic Assays

This invention further relates to the use of polynucleotides of the present invention as diagnostic reagents. Detection of a mutated form of the gene characterised by the polynucleotide of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO.37, SEQ ID NO.39, SEQ ID NO.41, SEQ ID NO.43, SEQ ID NO.45, SEQ ID NO.47 or SEQ ID NO:49 which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

It will thus be appreciated that this invention provides a method of diagnosing a pathological condition or a susceptibility to a pathological condition in a subject related to a disorder of CRH activity comprising:

(a) determining the presence or absence of a mutation in the polynucleotide according to the invention; and

(b) diagnosing a pathological condition or susceptibility to a pathological condition based on the presence or absence of said mutation.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled mammalian purine permease nucleotide sequences. Perfectly matched sequences can be distinguished from mismatched duplexes by RNase digestion or by differences in melting temperatures. DNA sequence differences may also be detected by alterations in electrophoretic mobility of DNA fragments in capilary electrophoresis columns or gels, with or without denaturing agents, or by direct DNA sequencing (e.g., Myers et al., Science (1985)230:1242). Sequence changes at specific locations may also be revealed by specific restriction endonucleases, nuclease protection assays, such as RNase and S1 protection or a chemical cleavage method (see Cotton et al., Proc Natl Acad Sci USA (1985) 85: 4397-4401). In another embodiment, an array of oligonucleotides probes comprising a nucleotide sequence encoding a protein capable of modulating CRH activity or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to the diseases through detection of mutations in the CRH modulating protein-encoding gene by the methods described. In addition, such diseases may be diagnosed by methods comprising determining from a sample derived from a subject an abnormally decreased or increased level of polypeptide or mRNA, as well as by determining from said samples the presence of protein derivatives compared to the normal structure. Decreased or increased expression can be measured at the RNA level using any of the methods well known in the art for the quantitation of polynucleotides, such as, for example; nucleic acid amplification, for instance via PCR, RT-PCR; RNase protection; Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays. Assay techniques that can be used to determine the presence of protein derivatives or variants comprise amongst others mass spectrometry.

Thus in another aspect, the present invention provides a method of diagnosing a pathological condition or a susceptibility to a pathological condition in a subject related to a disorder of prolonged CRH exposure comprising:

(a) determining the presence or amount of expression of the polypeptide or a derivative thereof according to the invention in a biological sample; and

(b) diagnosing a pathological condition or susceptibility to a pathological condition based on the presence or amount of expression of the polypeptide or of a derivative thereof.

In particular the present invention provides a method of diagnosing a CRH induced gene expression profile in an individual, said method comprising;
a) obtaining a biological sample of said individual; and
b) determine the amount of at least one protein that modulates corticotropin releasing hormone (CRH) signaling in said biological sample;
whereby the protein that modulates corticotropin releasing hormone (CRH) signaling is being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48. In an alternative embodiment the method of diagnosing a CRH induced expression profile is not limited to at least one protein according to the invention, but requires the simultaneous assessment of the expression levels of the group of proteins identified as being involved in CRH signaling, i.e. the proteins having the amino acid sequences SEQ ID NO.2, SEQ ID NO.4, SEQ ID NO.6, SEQ ID NO.8, SEQ ID NO.10, SQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44 and SEQ ID NO.48.

Preferably the amount of said proteins is determined either at the protein level, preferably using antibodies that bind thereto, or at the gene transcription level, preferably using probes that bind to a polynucleotide encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48. In the simultaneous assessment of the group of proteins involved in CRH signaling the level of gene expression is analysed using microarray technology.

Alternatively the CRH induced gene expression profile is determined by assessing the level of gene transcription of a gene comprising a nucleic acid sequence selected from the group consisting of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO.29, SEQ ID NO.31, SEQ ID NO.33, SEQ ID NO.35, SEQ ID NO.39, SEQ ID NO.41, SEQ ID NO.43, SEQ ID NO.45, SEQ ID NO.47 or SEQ ID NO:49. Methods to determine the level of gene transcription have been described hereinbefore and comprise in a preferred embodiment the use of a probe which binds, preferably selectively binds to a polynucleotide selected from the group consisting of SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO.29, SEQ ID NO.31, SEQ ID NO.33, SEQ ID NO.35, SEQ ID NO.37, SEQ ID NO.39, SEQ ID NO.41, SEQ ID NO.43, SEQ ID NO.45, SEQ ID NO.47 or SEQ ID NO:49 or the complement thereof. In another embodiment, an array of oligonucleotides probes comprising a nucleotide sequence according to the invention or fragments thereof can be constructed to conduct efficient screening of the level of gene transcription in the sample of an individual.

In a further aspect, the present invention relates to a diagnostic kit which comprises:

(a) a polynucleotide of the present invention, preferably the nucleotide sequence of SEQ ID NO: 45, SEQ ID NO: 47, SEQ ID NO: 49 or a fragment thereof;

(b) a nucleotide sequence complementary to that of (a);

(c) a polypeptide of the present invention, preferably the polypeptide of SEQ ID NO:46, SEQ ID NO 48 or a fragment thereof; or

(d) an antibody to a polypeptide of the present invention, preferably to the polypeptide of SEQ ID NO:46 or SEQ ID NO 48 and optionally suitable means for detection.

It will be appreciated that in any such kit, (a), (b), (c) or (d) may comprise a substantial component. Such a kit will be of use in diagnosing a disease or suspectability to a disease, particularly CRH metabolism related disorders such as CRH induced stress or depression.

The nucleotide sequences of the present invention are also valuable for chromosome localisation. These sequences are specifically targeted to, and can hybridize with, a particular location on an individual human chromosome. The mapping of relevant sequences to chromosomes according to the present invention is an important first step in correlating those sequences with gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes). The gene of the present invention maps to human chromosome 15.

The differences in the cDNA or genomic sequence between affected and unaffected individuals can also be determined. If a mutation is observed in some or all of the affected individuals but not in any normal individuals, then the mutation is likely to be the causative agent of the disease.

The nucleotide sequences of the present invention are also valuable for tissue localisation. Such techniques allow the determination of expression patterns of the polypeptides according to the invention in tissues by detection of the mRNAs that encode them. These techniques include in situ hybridziation techniques and nucleotide amplification techniques, for example PCR. Such techniques are well known in the art. Results from these studies provide an indication of the normal functions of the polypeptides in the organism.

The polypeptides of the invention or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Thus in another embodiment, this invention provides a monospecific antibody immunologically reactive with a mammalian purine permease. In a preferred embodiment said antibody is immunologically reactive with a polypeptide having an amino acid sequence selected from a group consisting of: (SEQ ID NO 46); (SEQ ID NO:48); and functional analogs thereof or said antibody blocks activity of a protein that modulates CRH signalling.

Antibodies generated against polypeptides of the present invention may be obtained by administering the polypeptides or epitope-bearing fragments, analogs or cells expressing these to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975)256:495-497), the trioma technique, the human B-cell hybridoma technique (Kozbor et al., Immunology Today (1983)4:72) and the EBV-hybridoma technique (Cole et al., MONOCLONAL ANTIBODIES AND CANCER THERAPY, pp. 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography.

Antibodies against polypeptides of the present invention may also be employed to treat the CRH metabolism related disorders.

In a further aspect, the present invention relates to genetically engineered soluble fusion proteins comprising a polypeptide of the present invention, or a fragment thereof, and various portions of the constant regions of heavy or light chains of immunoglobulins of various subclasses (IgG, IgM, IgD, IgE). Preferred as an immunoglobulin is the constant part of the heavy chain of human IgG, particularly IgG I, where fusion takes place at the hinge region. In a particular embodiment, the Fc part can be removed simply by incorporation of a cleavage sequence which can be cleaved with for instance blood clotting factor Xa. Furthermore, this invention relates to processes for the preparation of these fusion proteins by genetic engineering, and to the use thereof for drug screening, diagnosis and therapy. A further aspect of the invention also relates to polynucleotides encoding such fusion proteins. Examples of fusion protein technology can be found in International Patent Application Nos. W094/29458 and W094/22914.

Therapeutic Utility

A further aspect of the invention relates to an immunological/vaccine formulation (composition) which, when introduced into a mammalian host, induces an immunological response in that mammal to a polypeptide of the present invention wherein the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

In still another approach, expression of the gene encoding proteins which modulate CRH signalling can be inhibited using expression blocking techniques. Known such techniques involve the use of antisense sequences, either internally generated or externally administered (see, for example, O'Connor, J. Neurochem (1991) 56:560; Oligodeoxynucleotides as Antisense Inhibitors of Gene Expression, CRC Press, Boca Raton, Fla. (1988)). Alternatively, oligonucleotides which form triple helices (“triplexes”) with the gene can be supplied (see, for example, Lee et al., Nucleic Acids Res (1979)6:3073; Cooney et al., Science (1988)241:456; Dervan et al., Science (1991)251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these and/or other modified backbones also form part of the present invention.

In another process for inhibiting expression of a target gene in a cell, RNA with partial or fully double-stranded character is introduced into the cell or into the extracellular environment. Inhibition is specific in that a nucleotide sequence from a portion of the target gene is chosen to produce inhibitory RNA. The RNA may comprise one or more strands of polymerized ribonucleotide; it may include modifications to either the phosphate-sugar backbone or the nucleoside. The double-stranded structure may be formed by a single self-complementary RNA strand or two complementary strands. Inhibition is sequence-specific in that the nucleotide sequences corresponding to the duplex region of the RNA are targeted for genetic inhibition. RNA containing a nucleotide sequence identical to a portion of the target sequence is preferred. Examples of RNA inhibition technology can be found in International Patent Application WO 99/32619.

In addition, expression of the proteins which modulate CRH signalling may be prevented by using ribozymes specific to the mRNA sequence encoding said protein. Ribozymes are catalytically active RNAs that can be natural or synthetic (see for example Usman, N, et al., Curr. Opin. Struct. Biol (1996)6(4), 527-33.) Synthetic ribozymes can be designed to specifically cleave the aforementiond mRNAs at selected positions thereby preventing translation of said mRNAs into functional polypeptide. Ribozymes may be synthesised with a natural ribose phosphate backbone and natural bases, as normally found in RNA molecules. Alternatively the ribozymes may be synthesised with non-natural backbones to provide protection from ribonuclease degradation, for example, 2′-O-methyl RNA, and may contain modified bases.

For treating abnormal conditions related to an under-expression of proteins which modulate CRH signalling, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Alternatively, gene therapy may be employed to effect the endogenous production of mammalian purine permease by the relevant cells in the subject. For example, a polynucleotide of the invention may be engineered for expression in a replication-defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of patches, salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXPERIMENTAL PROCEDURES

Cell culture and sample preparation—AtT-20 cells were purchased from ATCC and were maintained at 37° C. in 5% CO₂ in humidified air in Dulbecco's modified Eagle's medium (Invitrogen Life Technologies) containing 10% fetal bovine serum, 5% horse serum and 4.5 g/L D-glucose. For experiments cells were seeded in 25 cm² flasks. The medium was replaced 48 h later and cells were treated with either 0.1% DMSO, 1 μM CRH (Sigma) in DMSO, 1 μM R121919 in DMSO or 1 μM CRH+1 μM R121919 in DMSO for 0, 0.5, 1, 2, 4, 8 and 24 h in fresh medium. The incubation was stopped by aspirating the incubation medium and adding 3 ml Trizol (Invitrogen Life Technologies) for lysis of the cells. Total RNA was extracted using Trizol according to the instructions of the manufacturer. 100 μg total RNA was further purificied using Rneasy kit (Qiagen) with DNAseI treatment on column.

Microarray hybridization—cRNA was prepared as follows. Reverse transcription was performed on 10 μg of total RNA for 1 h at 42° C. using a T7-oligo(dT)₂₄-primer and SuperscriptII RT (Invitrogen Life Technologies). Second strand cDNA synthesis was done for 2 h at 16° C. using Escherichia coli DNA Polymerase I, DNA ligase and RNAseH (Invitrogen Life Technologies). After phenol-chloroform extraction using phase-lock gel (Eppendorf) in vitro transcription was performed for 6 h at 37° C. using the Bioarray high-yield RNA transcript labeling kit with Biotin labeled ribonucleotides (Enzo Diagnostics). cRNA samples were purified on Qiagen Rneasy columns followed by fragmentation for 35 min at 95° C. cRNA yields were between 50 and 100 μg.

Samples were processed on GeneChips (Affymetrix, Santa Clara, Calif.). In order to check the quality of each sample, 5 μg of labeled cRNA was run on Test2-arrays. Actual experiments were performed on Murine Genome U74Av2 arrays, containing probe sets interrogating approximately 12,000 full-length mouse genes and EST clusters from the UniGene database (Build 74). Hybridization was performed using 15 μg cRNA for 16 h at 45° C. under continuous rotation. Arrays were stained in Affymetrix Fluidics stations using Streptavidin/Phycoerythrin (SAPE) followed by staining with anti-streptavidin antibody and a second SAPE staining. Subsequently arrays were scanned with a HP-Laserscanner and data were analyzed with the Microarray Suite Software (Affymetrix). No scaling or normalization was performed at this stage. Quality of the experiment was assessed based on the percentages present calls across all samples which was on average 47.06±2.45%. The cytoplasmic β-actin and GAPDH 5′/3′ ratios were 1.10±0.08 and 0.93±0.05 respectively.

Data Analysis and Selection of Genes

Raw intensities on each array were aligned using the following global mean algorithm:

$$log {I}_{aligned} = log {I}_{raw} + ( log \frac{{\sum }_{all samples} {I}_{raw}}{\# genes \times \# samples} - log \frac{{\sum }_{sample} {I}_{raw}}{\# genes} )$$

Basically this alignment sets the average intensity of one array to the average measured across all arrays, compensating for array to array variations in hybridization, washing and staining, ultimately allowing a reasonable comparison between arrays. After alignment polished data were analyzed using weighted spectral mapping. Weighted spectral mapping is an unsupervised multivariate analysis method which includes double-centering of the data combined with a specialized visualization representing the two highest principle components. Even though double-centering removes the “size” component of the array data, this information is reintroduced in the visualization via the area of the symbols representing the size of the respective samples and genes. This method allows the reduction of a large microarray dataset and provides means to visually inspect and thereby identify clusters of genes and/or subjects in the data (7). A more detailed analysis was carried out using the OmniViz program. All records which were absent in all experiments were removed and all signals less than 20 were set to 20. Gene expression fold differences for CRH, R121919 and CRH+R121919 treatments were calculated at each timepoint. For those calculations signals at corresponding timepoints in DMSO treated samples were used to calculate ratios.

Quantitative RT-PCR—Microarray data were confirmed using real time PCR analysis. First strand cDNA synthesis was performed on 0.5 μg total RNA using random hexamer primers and SuperscriptII RT (Invitrogen Life Technologies). Quantitative PCR was performed on a ABIPrism 7700 cycler (Applied Biosystems) using a Taqman PCR kit. Serial dilutions of cDNA were used to generate standard curves of threshold cycles versus the logarithms of concentration for β-actin, c-fos, Crh-R1, Crh-R2, Rgs2 and the genes of interest (see table 2 for sequences). A linear regression line calculated from the standard curves allowed the determination of transcript levels in RNA samples from the different time points.

### RESULTS

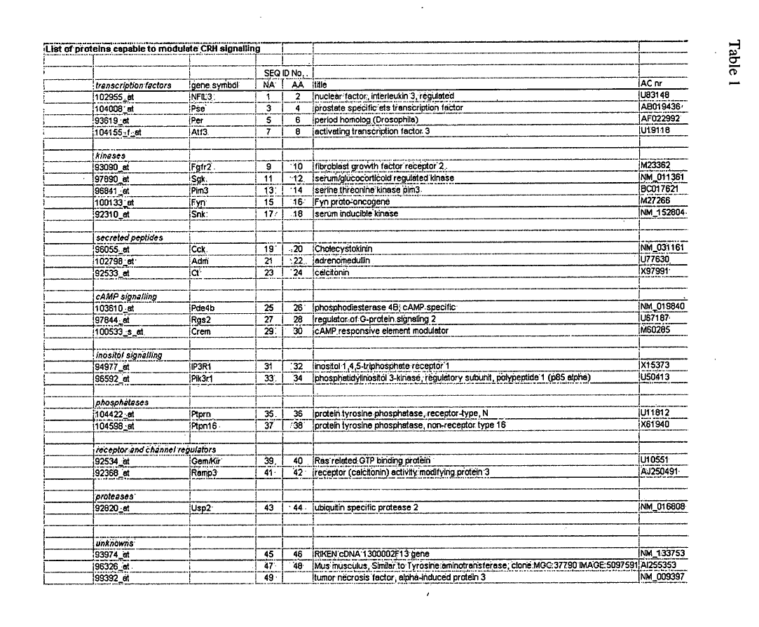

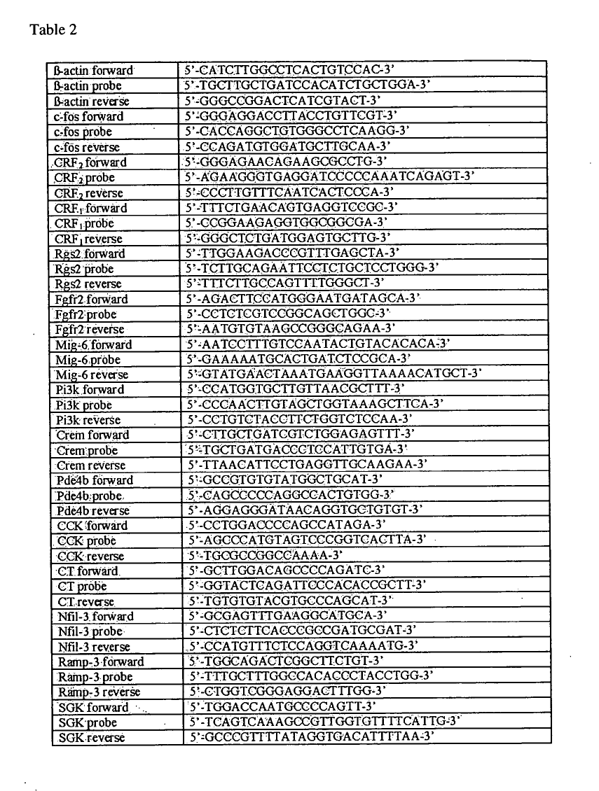

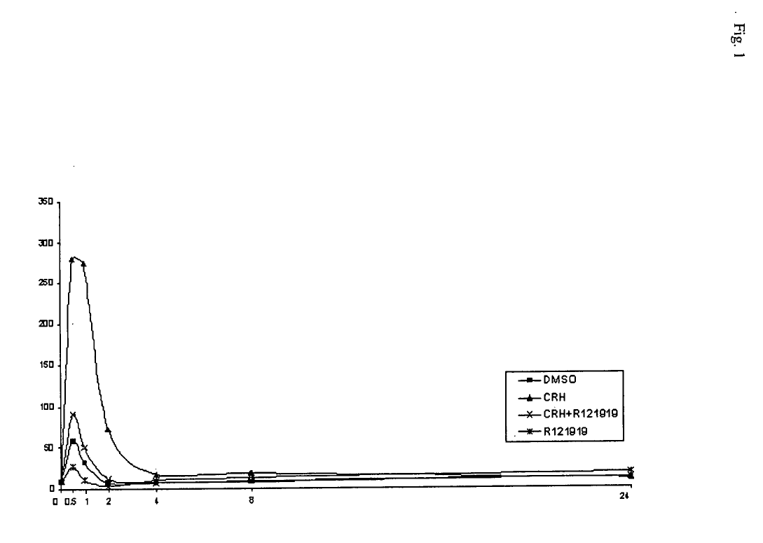

Transcriptional response to CRH was studied in the CRH-R1-expressing murine AtT-20 pituitary corticotroph-derived adenoma cell line. Whereas CRH-R1 was readily detectable both by real time quantitative RT-PCR (RTq) and western blot, CRH-R2 expression could not be discerned in AtT-20 cells. In order to identify CRH-R1 specific responses cells were exposed to 1 μM CRH, 1 μM CRH in the presence of 1 μM of a CRH-R1 specific antagonist R121919 and to R121919 alone. Transcriptional responses were followed over time until 24 h after the first administration. In order to assess treatment efficacy, c-fos mRNA levels were determined by RTq on RNA from the different treatments and time points before array experiments were carried out. In agreement with previous reports, exposure to CRH elicited a transient surge in c-fos transcription, with levels already going down after 0.5 to 1 h (see FIG. 1)(8;9). This response was almost completely suppressed in the presence of R121919. Interestingly, 0.1% DMSO induced c-fos expression, however levels were between 5 to 10 times lower compared to CRH induced expression.

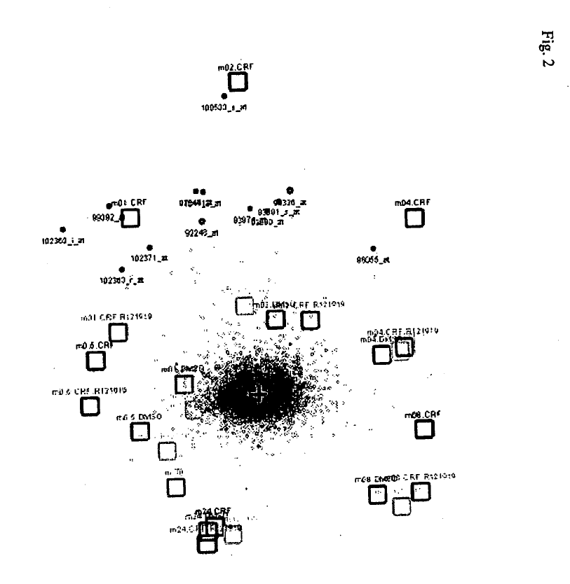

All time points were analyzed on microarrays containing approximately 12000 murine genes and ESTs. Overall analysis of the expression profiles using spectral mapping (a so called unsupervised method) indicated progressing time to account for most of the observed changes in gene expression (see FIG. 2). Synchronization of cell cycle in these cultures induced by addition of new medium and serum could possibly account for this phenomenon although accumulation of metabolites and progressing cell culture are additional contributing factors. The spectral map analysis also showed that CRH treated samples differed from other samples mainly in the early time points (0.5 h until 2 h), with overall differences in expression becoming very small after 8 h. Because of this obvious influence of time, expression measurements were analyzed relative to those observed in the corresponding time point in DMSO treated control samples.

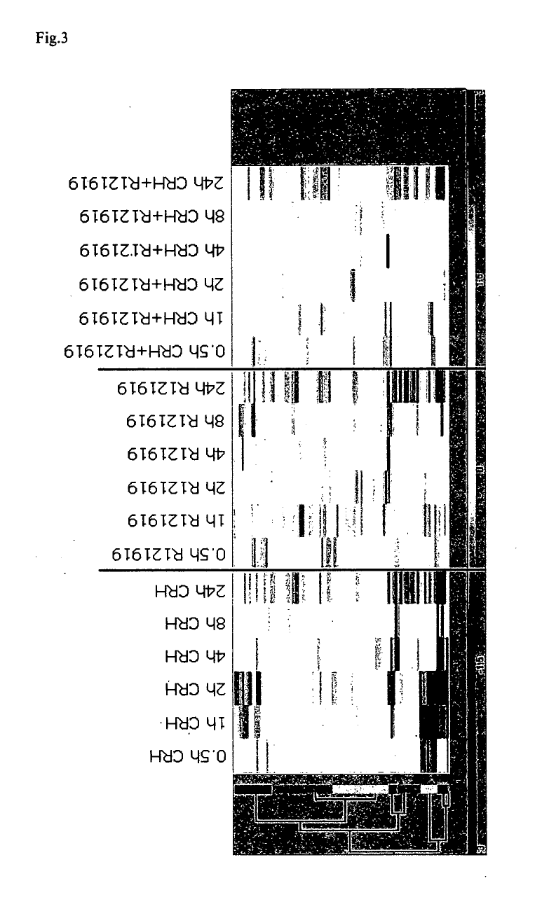

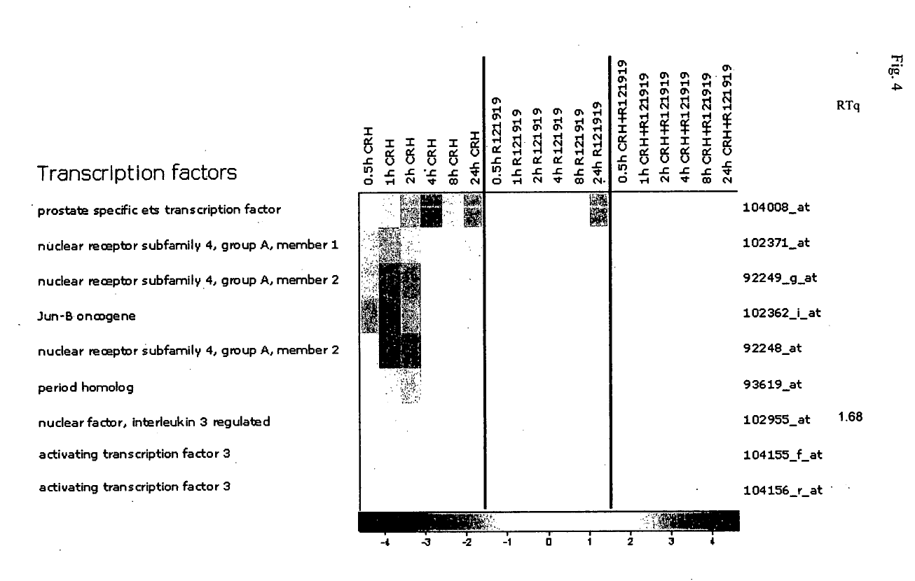

Regulated genes were defined as those showing a greater than 2-fold change in transcript levels at any one time point. Using the OmniViz Treescape view, 111 genes that met this criterion, showing a difference in expression after treatment with CRH compared to treatment with the antagonist, were selected. 26 out of the 111 genes were “early responders”, showing a 2-fold change already after 30 minutes treatment with CRH. 32 genes were “intermediate responders”, responding after 1 to 2 hours of treatment and 53 genes were “late responders”, showing a response after 2 hours or more after treatment (see FIG. 3). These responses were suppressed by the CRH-R1 antagonist R121919. Among the early responders were known players in the pathways downstream of the CRH-R1 such as the transcription factors Nurr1, Nurr77, Jun-B, validating the assay.

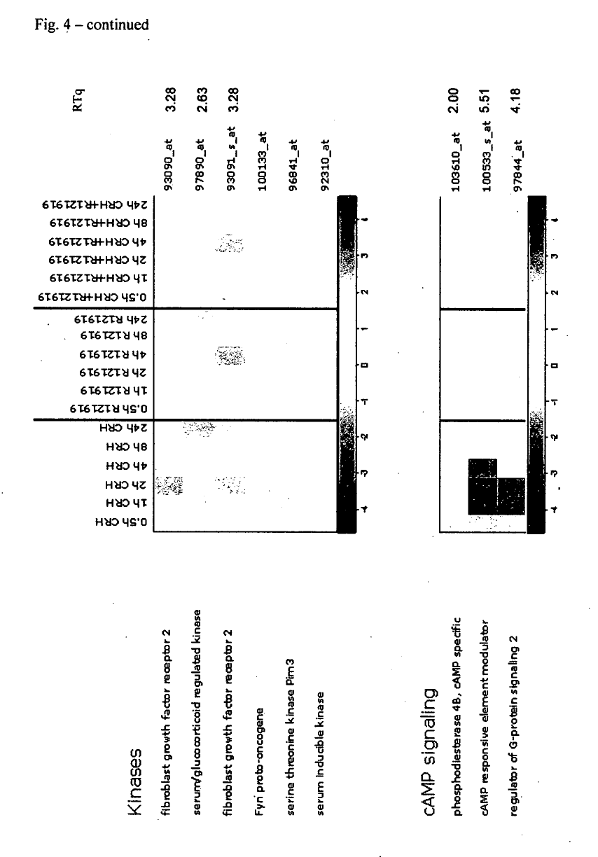

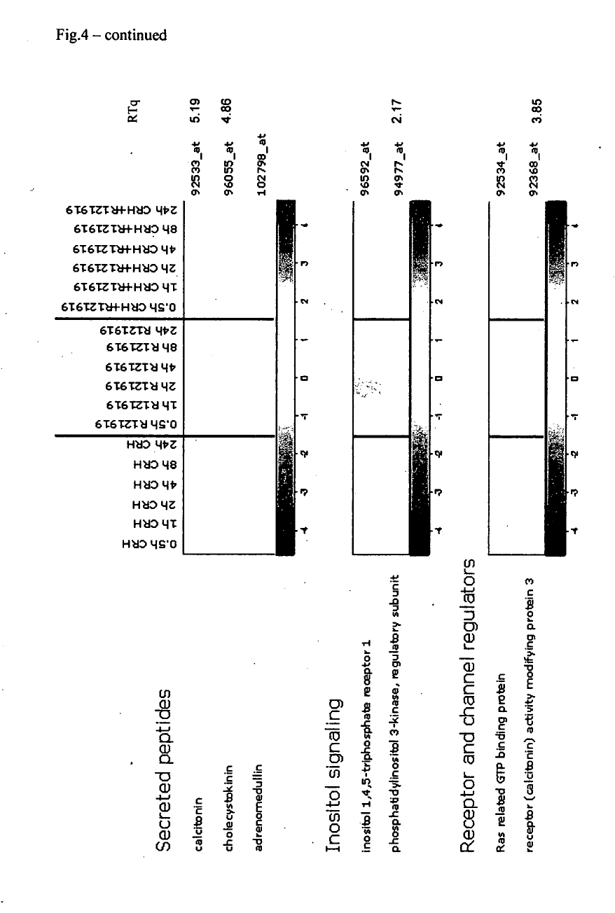

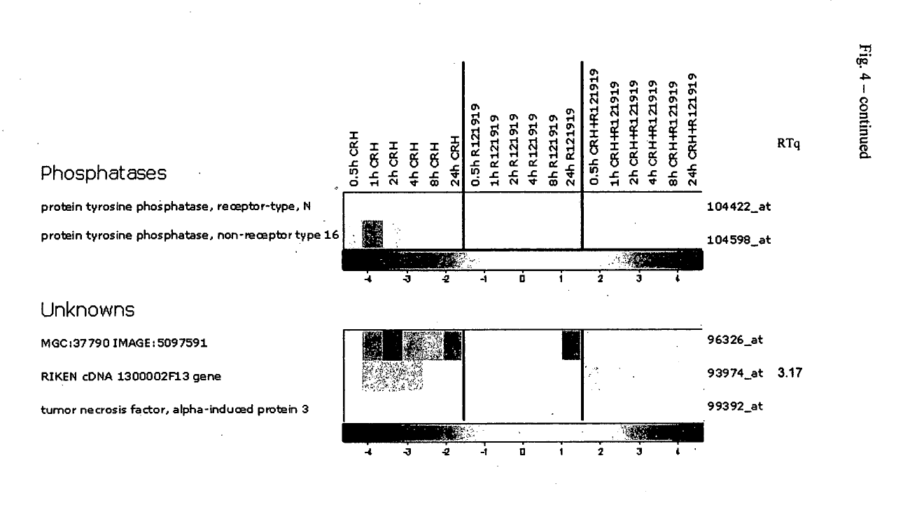

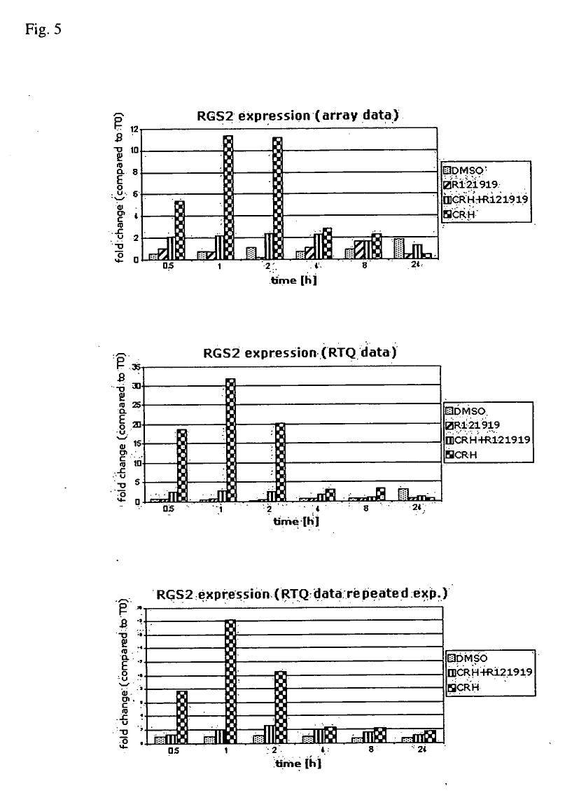

Interesting novel players identified include transcription factors (e.g. hairy/enhancer-of-split related 1 (Hey1), nuclear factor regulated by interleukin 3 (NFIL3), cAMP responsive element modulator (CREM) and prostate specific ets transcription factor (Pse)), receptor and channel regulators (e.g. Ras-related GTP-binding protein (GEM) and receptor (calcitonin) activity modifying protein 3 (RAMP3)), secreted peptides (e.g. adrenomedullin, calcitonin, cholecystokinin) and proteins involved in intracellular signaling (e.g. regulator of G-protein signaling 2 (Rgs2), cAMP specific phosphodiesterase 4B (Pde4b), inositol 1,4,5-triphosphate receptor 1 (IP₃R1) and the regulatory subunit phosphatidylinositol 3-kinase, p85). Other interesting regulated genes comprise Period homolog Per1, fibroblast growth factor receptor 2 (Fgfr2), serum/glucocorticoid regulated kinase and serum-inducible kinase (FIG. 4). Interestingly, all responders identified according to above mentioned criteria were up regulated after exposure to CRH. This induction was transient and nearly all of the induced genes return to baseline after 4 to 8 hr. Many of the induced transcripts encode proteins that would exert a negative feedback on the CRH-R1 signaling (e.g. Pde4, Rgs2, CREM, etc . . . ), possibly contributing to the transient nature of the induction. In additiob to this negative feedback, other mechanisms such as desentisation of CRF₁ through phosphorylation and internalization contribute to the transient nature of the transcriptional induction. In this respect it is of interest to note that challenge with CRF quickly downregulates CRF₁ mRNA in rat pituitary cells. We could however not detect any alterations in CRF₁ mRNA in pituitary derived AtT-20 cells exposed to 1 μM CRF. Confirmation of microarray data was carried out using quantitative real time PCR analysis on the same samples used for hybridization experiments and on a repeated experiment. Levels of regulation and the time course identified by microarray corresponded to those observed by quantitative PCR as shown for Rgs2 in FIG. 5. For those genes that have been tested, levels iof induction compared to the untreated samples are indicated in FIG. 4.

### DISCUSSION

We have identified transcriptional pathways downstream of the CRH-R1 in AtT-20 cells using a CRH-R1 specific antagonist. Our findings are in agreement with activation of several second messenger such as cAMP and Ca²⁺ upon stimulation with CRH. Some of the transcriptional responses can be explained by the phosphorylation of CREB and the subsequent transcription of genes downstream of cAMP responsive elements. These elements have been found for example in the promoters of Per1, Nurr1, CREM-ICER, c-Fos. Furthermore the kinetic profile of the induction of these genes correspond with the observed maximal transcription rate by CREB after 0.5 hr of cAMP formation. The induction of CREM-ICER constitutes a negative feedback mechanism in attenuating transcriptional response to cAMP. Of interest is the reported induction of CREM-ICER in response to acute stress in the intermediate lobe of the pituitary gland. Mice deficient for CREM-ICER show a chronic increase of beta-endorphin levels suggesting that CREM-ICER induction may be involved in the modulation of gene expression in response to stress (10). Our results suggest that CREM-ICER is directly involved in the modulation of CRH signaling and as a result, ablation of CREM-ICER could lead to an altered response to stress signals. Another novel putative negative feedback regulator of CRH signaling is Rgs2. We identified two single CRE motifs in the promoter of the human RGS2 gene, providing a possible explanation for the early response behavior of this gene upon stimulation with CRH. In support of our findings is a recent report showing that both phosphoinositide signaling and cAMP induce a rapid and transient increase in Rgs2 mRNA in human astrocytoma and neuroblastoma cells. The Rgs2 protein is a selective inhibitor of G_{qα} function. Recently it has been shown that Rgs2 reduces odorant-elicited cAMP production, not by acting on G_{α} but by directly inhibiting the activity of adenylyl cyclase type III. Although Rgs2 was originally identified as an immediate early response gene in activated T lymphocytes, studies in Rgs2 deficient mice indicate that it also plays a role in the modulation of stress related behavior as these mice show increased anxiety and aggression (11). Also the induction of cAMP specific phosphodiesterase 4B (Pde4b) can be categorized under negative feedback, directly attenuating the cAMP signal. Another important second messenger generated upon stimulation with CRH is Ca²⁺. It has been shown that CRH triggers a steady-state depolarization stimulated extracellular Ca²⁺ entry via voltage-gated Ca²⁺ channels and raises intracellular Ca²⁺ concentration through release from inositol 1,4,5-triphosphate (InsP3) sensitive Ca²⁺ pools (12). Both the InsP3 receptor and the p85 regulatory subunit of phosphatidylinositol 3-kinase are upregulated, possibly accounting for a compensating mechanism for prolonged Ca²⁺ signaling. Also the upregulation of the small G-protein kir/Gem points towards an attenuation of prolonged Ca²⁺ signaling. Recent studies have shown that Gem regulates Ca²⁺ channel expression at the cell membrane through the β auxiliary subunits. Increased levels of Gem have been shown to inhibit Ca²⁺-triggered exocytosis and it has been proposed that Gem could have a protective effect against Ca²⁺ overload. In addition to its role as second messenger, intracellular Ca²⁺ has also been shown to play a critical role in regulating gene expression. Of interest is the regulation of NFIL3/E4BP4 by calcineurin/NFAT and CaM kinase signaling, accounting for an increase in NFIL3 mRNa levels upon CRH treatment. In B lymphocytes expression of NFIL3 is induced by interleukin 3 through both the Raf-mitogen-activated protein kinase and phosphatidylinositol 3-kinase pathways. In this cell type NFIL3 inhibits apoptosis in synergy with Bcl-xL dependent pathways. Our data suggest a role for NFIL3 in prevention of apoptosis in AtT-20 cells.

CRH is the most efficacious ACTH secretagogue. Unfortunately the microarrays that were used did not interrogate for POMC levels. However several other prepropeptides mRNAs were found upregulated after CRH administration such as cholecystokinin (CCK) and two calcitonin peptide family members, adrenomedullin (ADM) and calcitonin (CT). Also of interest in this respect is the upregulation of RAMP3. RAMPs control the transport and glycosylation of the calcitonin receptor-like receptor (CRLR). In the case of RAMP3, it has been shown that together with CRLR it generates an ADM receptor. Upregulation of this gene might play a role in regulating the responsiveness of AtT-20 cells to ADM after CRH exposure or to other extracellular stimuli as it is not known whether RAMP3 might regulate other G-coupled receptors. Although CCK is secreted by AtT-20 cells, induction of its expression by CRH has not been previously reported (13). Interaction between CCK and CRH has however been intensively studied and demonstrated in panic attacks, depression, anxiety and gastric emptying (14-19). Most of these experiments point towards a role for CRH in mediating the central effects of CCK. Our data indicate that CRH in addition might function as a CCK secretagogue. A very similar situation to that of CCK seems to be case for adrenomedullin as well. It has been demonstrated that ADM is expressed in pituitary gland and affects basal and CRH-stimulated ACTH release in animals, thus suggesting its potential role in regulating the hypothalamus-pituitary-adrenal axis (20-23). Current expression data show that CRH induces ADM and CT. In addition recent findings indicate that circulating adrenomedullin is increased in Cushing's disease, and the pituitary gland may represent the site of the elevated production of ADM (24), suggesting CRH might be inducing ADM.

In conclusion, we have unraveled part of the corticotropin-releasing hormone receptor-1 activated gene network and have identified several novel targets of this signaling cascade. Our findings trigger the need for further experiments to elucidate the function of these transcriptional responses to CRH stimulation both on a cellular and whole organism level.

### REFERENCES

1. De Souza E B 1995 Corticotropin-releasing factor receptors: physiology, pharmacology, biochemistry and role in central nervous system and immune disorders. Psychoneuroendocrinology 20:789-819
2. Holsboer F 2001 Stress, hypercortisolism and corticosteroid receptors in depression: implications for therapy. J Affect Disord 62:77-91
3. Holsboer F, Gerken A, Stalla G K, Muller O A 1987 Blunted aldosterone and ACTH release after human CRH administration in depressed patients. Am J Psychiatry 144:229-231
4. Holsboer F, Gerken A, von Bardeleben U, Grimm W, Beyer H, Muller O A, Stalla G K 1986 Human corticotropin-releasing hormone in depression—correlation with thyrotropin secretion following thyrotropin-releasing hormone. Biol Psychiatry 21:601-611
5. Nemeroff C B, Owens M J, Bissette G, Andorn A C, Stanley M 1988 Reduced corticotropin releasing factor binding sites in the frontal cortex of suicide victims. Arch Gen Psychiatry 45:577-579
6. Raadsheer F C, Hoogendijk W J, Stam F C, Tilders F J, Swaab D F 1994 Increased numbers of corticotropin-releasing hormone expressing neurons in the hypothalamic paraventricular nucleus of depressed patients. Neuroendocrinology 60:436-444
7. Wouters L, Göhlmann H W, Bijnens L, Kass S U, Molenberghs G, Lewi P J 2002 Graphical exploration of gene expression data: a comparative study of three multivariate methods. Biometrics
8. Boutillier A L, Monnier D, Lorang D, Lundblad J R, Roberts J L, Loeffler J P 1995 Corticotropin-releasing hormone stimulates proopiomelanocortin transcription by cFos-dependent and -independent pathways: characterization of an API site in exon 1. Mol Endocrinol 9:745-755
9. Boutillier A L, Sassone-Corsi P, Loeffler J P 1991 The protooncogene c-fos is induced by corticotropin-releasing factor and stimulates proopiomelanocortin gene transcription in pituitary cells. Mol Endocrinol 5:1301-1310
10. Mazzucchelli C, Sassone-Corsi P 1999 The inducible cyclic adenosine monophosphate early repressor (ICER) in the pituitary intermediate lobe: role in the stress response. Mol Cell Endocrinol 155:101-113
11. Oliveira-Dos-Santos A J, Matsumoto G, Snow B E, Bai D, Houston F P, Whishaw I Q, Mariathasan S, Sasaki T, Wakeham A, Ohashi P S, Roder J C, Barnes C A, Siderovski D P, Penninger J M 2000 Regulation of T cell activation, anxiety, and male aggression by RGS2. Proc Natl Acad Sci USA 97:12272-12277
12. Tse A, Lee A K 2000 Voltage-gated Ca2+ channels and intracellular Ca2+ release regulate exocytosis in identified rat corticotrophs. J Physiol 528 Pt 1:79-90.:79-90
13. Beinfeld M C 1992 CCK mRNA expression, pro-CCK processing, and regulated secretion of immunoreactive CCK peptides by rat insulinoma (RIN 5F) and mouse pituitary tumor (AtT-20) cells in culture. Neuropeptides 22:213-217
14. Coskun T, Bozkurt A, Alican 1, Ozkutlu U, Kurtel H, Yegen B C 1997 Pathways mediating CRF-induced inhibition of gastric emptying in rats. Regul Pept 69:113-120
15. Kellner M, Wiedemann K, Yassouridis A, Levengood R, Guo L S, Holsboer F, Yehuda R 2000 Behavioral and endocrine response to cholecystokinin tetrapeptide in patients with posttraumatic stress disorder. Biol Psychiatry 47:107-111
16. Geracioti T D, Jr., Ekhator N N, Nicholson W E, Arndt S, Loosen P T, Orth D N 1999 Intra- and inter-individual correlations between cholecystokinin and corticotropin-releasing hormone concentrations in human cerebrospinal fluid. Depress Anxiety 10:77-80
17. Calogero A E, Nicolosi A M, Moncada M L, Coniglione F, Vicari E, Polosa P, D'Agata R 1993 Effects of cholecystokinin octapeptide on the hypothalamic-pituitary-adrenal axis function and on vasopressin, prolactin and growth hormone release in humans. Neuroendocrinology 58:71-76
18. Biro E, Sarnyai Z, Penke B, Szabo G, Telegdy G 1993 Role of endogenous corticotropin-releasing factor in mediation of neuroendocrine and behavioral responses to cholecystokinin octapeptide sulfate ester in rats. Neuroendocrinology 57:340-345
19. Kamilaris T C, Johnson E O, Calogero A E, Kalogeras K T, Bernardini R, Chrousos G P, Gold P W 1992 Cholecystokinin-octapeptide stimulates hypothalamic-pituitary-adrenal function in rats: role of corticotropin-releasing hormone. Endocrinology 130:1764-1774
20. Shan J, Krukoff T L 2001 Intracerebroventricular adrenomedullin stimulates the hypothalamic-pituitary-adrenal axis, the sympathetic nervous system and production of hypothalamic nitric oxide. J Neuroendocrinol 13:975-984
21. Martinez V, Cuttitta F, Tache Y 1997 Central action of adrenomedullin to inhibit gastric emptying in rats. Endocrinology 138:3749-3755
22. Parkes D G, May C N 1995 ACTH-suppressive and vasodilator actions of adrenomedullin in conscious sheep. J Neuroendocrinol 7:923-929
23. Samson W K, Murphy T, Schell D A 1995 A novel vasoactive peptide, adrenomedullin, inhibits pituitary adrenocorticotropin release. Endocrinology 136:2349-2352
24. Letizia C, Di Iorio R, De Toma G, Marinoni E, Cerci S, Celi M, Subioli S, D'Erasmo E 2000 Circulating adrenomedullin is increased in patients with corticotropin-dependent Cushing's syndrome due to pituitary adenoma. Metabolism 49:760-763

1. An isolated polynucleotide encoding a protein, which modulates corticotropin releasing hormone (CRH) signaling said protein having an amino acid sequence selected from the group consisting of SEQ ID NO.: 46 and SEQ ID NO.: 48.

2. An isolated polynucleotide according to claim 1 wherein said polynucleotide is mRNA, DNA or cDNA.

3. An isolated polynucleotide encoding a protein, which modulates corticotropin releasing hormone (CRH) signaling said polynucleotide comprising a nucleic acid sequence selected from the group consisting of SEQ ID NO. 45, SEQ ID NO.: 47 and SEQ ID NO.: 49.

4. An isolated polynucleotide encoding a protein, which modulates corticotropin releasing hormone (CRH) signaling said polynucleotide consisting of a nucleic acid sequence selected from the group consisting of SEQ ID NO. 45, SEQ ID NO.: 47 and SEQ ID NO.: 49.

5. An isolated polypeptide encoding a protein, which modulates corticotropin releasing hormone (CRH) signaling said protein comprising an amino acid sequence selected from the group consisting of SEQ ID NO.: 46 and SEQ ID NO.: 48.

6. A vector comprising an isolated polynucleotide according to claim 1.

7. A vector according to claim 6 wherein the polynucleotide is operably linked to an expression control sequence.

8. A host cell capable of expressing a protein, which modulates corticotropin releasing hormone (CRH) signaling, said protein having an amino acid sequence selected from the group consisting of SEQ ID NO.: 46 and SEQ ID NO.: 48.

9. A host cell according to claim 8 transfected with a vector comprising a regulatory sequence.

10. A host cell according to claim 8 transfected with a vector according to claim 6.

11. A method for identifying a compound capable to alter the CRH signalling response in a cell, said method comprising;
a) contacting said cell with CRH in the presence and absence of said compound;
b) determine the amount of at least one protein that modulates corticotropin releasing hormone (CRH) signaling in said cell; and
c) compare the amount of said protein in the presence and absence of said compound;
whereby the protein that modulates corticotropin releasing hormone (CRH) signaling is being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

12. A method according to claim 11 wherein the cell is a eukaryotic cell such as the murine pituitary corticotroph-derived adenoma cell line AtT-20.

13. A method according to claim 11 wherein the amount of protein that modulates CRH signaling is being determined using an antibody which binds to a polypeptide comprising an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

14. A method according to claim 11 wherein the amount of protein that modulates CRH signaling is being determined by assessing the level of gene transcription of a gene encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

15. A method according to claim 14 wherein the level of gene transcription is being assessed using a probe which binds to a polynucleotide encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

16. A method according to claim 14 wherein the level of gene expression is analysed using microarray technology.

17. A method according to claim 16 wherein the level of gene expression is assessed using an array of oligonucleotide probes that bind to the polynucleotides encoding the group of polypeptides having the amino acid sequences SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

18. A method for identifying a compound capable to alter the CRH signalling response in a cell, said method comprising;
a) contacting said cell in the presence and absence of a test compound; and
b) determine the expression levels of the genes encoding the polypeptides with the amino acid sequences SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

19. A method according to claim 18 wherein the expression levels of the genes are determined using an array of oligonucleotide probes that bind to the polynucleotides having the nucleic acid sequences SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO.29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 and SEQ ID NO:49.

20. A method for identifying a compound capable to alter the CRH signalling response activity in a cell, said method comprising;
a) contacting a cell which expresses at least one protein comprising an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48, with said test compound; and
b) compare the CRH response activity of said cell in the presence and absence of said compound.

21. A method according to claim 20 wherein the cell expresses the group of proteins having the amino acid sequences SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

22. A method according to claim 21, wherein the CRH response activity is being assessed as changes in transcription at the gene level.

23. A method according to claim 21, wherein the CRH response activity is being assessed using microarray technology.

24. A method according to claim 20 wherein the cell is host cell capable to express at least one protein having an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

25. A method according to claim 24 wherein the host cell is transfected with at least one vector comprising a regulatory sequence.

26. A method according to claim 24 wherein the host cell is transfected with at least one vector comprising a polynucleotide sequence encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

27. A method of diagnosing CRH induced depression in an individual said method comprising;
a) obtaining a biological sample of said individual; and
b) determine the amount of at least one protein that modulates corticotropin releasing hormone (CRH) signaling in said biological sample;
whereby the protein that modulates corticotropin releasing hormone (CRH) signaling is being selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

28. A method according to claim 27 wherein the biological sample is a bodily fluid or tissue sample

29. A method according to claim 27 wherein wherein the amount of protein that modulates CRH signaling is being determined using an antibody which binds to a polypeptide comprising an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

30. A method according to claim 27 wherein the amount of protein that modulates CRH signaling is being determined by assessing the level of gene transcription of a gene encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

31. A method according to claim 30 wherein the level of gene transcription is being assessed using a probe which binds to a polynucleotide encoding an amino acid sequence selected from the group consisting of SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

32. A method according to claim 30 wherein the level of gene transcription is analysed using microarray technology.

33. A method according to claim 32 wherein the level of gene transcription is analysed using an array of oligonucleotide probes that bind to the polynucleotides encoding the group of polypeptides having the amino acid sequences SEQ ID NO.2, SEQ ID 4, SEQ ID NO.6, SEQ ID NO.8, SEQ ID NO.10, SEQ ID NO.12, SEQ ID NO.14, SEQ ID NO.16, SEQ ID NO.18, SEQ ID NO.20, SEQ ID NO.22, SEQ ID NO.24, SEQ ID NO.26, SEQ ID NO.28, SEQ ID NO.30, SEQ ID NO.32, SEQ ID NO.34, SEQ ID NO.36, SEQ ID NO.38, SEQ ID NO.40, SEQ ID NO.42, SEQ ID NO.44, SEQ ID NO.46 and SEQ ID NO.48.

34. A method according to claim 32 wherein the level of gene transcription is analysed using an array of oligonucleotide probes that bind to that bind to the polynucleotides having the nucleic acid sequences SEQ ID NO: 1, SEQ ID NO 3, SEQ ID NO.5, SEQ ID NO.7, SEQ ID NO.9, SEQ ID NO.11, SEQ ID NO.13, SEQ ID NO.15, SEQ ID NO.17, SEQ ID NO.19, SEQ ID NO.21, SEQ ID NO.23, SEQ ID NO.25, SEQ ID NO.27, SEQ ID NO. 29, SEQ ID NO.31, SEQ ID NO.33, SEQ ID NO.35, SEQ ID NO. 37, SEQ ID NO.39, SEQ ID NO.41, SEQ ID NO.43, SEQ ID NO.45, SEQ ID NO.47 and SEQ ID NO:49.
